(19) United States
(12) Patent Application Publication
(10) Pub. No.: US 2024/0301515 A1
(43) Pub. Date: Sep. 12, 2024

(54) DENDRIMERS FOR GENOMIC ANALYSIS METHODS AND COMPOSITIONS

(71) Applicant: Dovetail Genomics, LLC, Scotts Valley, CA (US)

(72) Inventor: Marco BLANCHETTE, Santa Cruz, CA (US)

(21) Appl. No.: 18/665,851

(22) Filed: May 16, 2024

Related U.S. Application Data
(63) Continuation of application No. PCT/US22/50291, filed on Nov. 17, 2022.
(60) Provisional application No. 63/281,544, filed on Nov. 19, 2021.

(51) Int. Cl. C12Q 1/689; C12Q 1/6806; C12Q 1/6874

(52) U.S. Cl. CPC C12Q 1/689; C12Q 1/6806; C12Q 1/6874; C12Q 2600/16

(57) ABSTRACT

Provided herein are methods and compositions for nucleic acid processing comprising obtaining a stabilized sample comprising a nucleic acid molecule complexed to at least one nucleic acid binding protein; contacting said stabilized sample to a dendrimer comprising a plurality of nucleic acid binding moieties such that said nucleic acid molecule forms a complex with said plurality of nucleic acid binding moieties; contacting said complex to an endonuclease to cleave said nucleic acid molecule between contact points of said nucleic acid molecule and said plurality of nucleic acid binding moieties creating a plurality of fragments of said nucleic acid molecule each complexed with a nucleic acid binding moiety of said plurality of said nucleic acid binding moieties; isolating said product using an agent that binds to said dendrimer; joining said plurality of fragments to each other to create a concatemer comprising each of said plurality of fragments of said nucleic acid molecule; and isolating said concatemer from said dendrimer.

### CROSS-REFERENCE

This application is a continuation of International Patent Application No. PCT/US22/50291, filed Nov. 17, 2022, which claims the benefit of U.S. Provisional Application No. 63/281,544, filed Nov. 19, 2021, which is incorporated herein by reference in its entirety.

### BACKGROUND

Dendrimers are highly ordered, branched polymers, usually highly symmetric and spherical. The properties of dendrimers are determined by functional groups found at the surface of the dendrimer. Dendrimers are also characterized by generation, referring to the number of repeated branching cycles performed during synthesis with higher generation dendrimers having more exposed functional groups at the surface.

### SUMMARY

In an aspect, there are provided compositions comprising a dendrimer comprising a plurality of nucleic acid binding moieties; and a plurality of nucleic acid fragments. In some cases, the plurality of nucleic acid fragments are derived from a common chromosome. In some cases, the plurality of nucleic acid fragments are derived from different chromosomes. In some cases, the plurality of nucleic acid fragments comprise cell-free nucleic acids. In some cases, the plurality of nucleic acid fragments are proximal to each other in a cell. In some cases, the dendrimer comprises poly(amidoamine) (PAMAM). In some cases, the dendrimer is about 3.2 kDa to about 116 kDa. In some cases, the dendrimer comprises about 16 to about 512 nucleic acid binding moieties. In some cases, the plurality of nucleic acid binding moieties comprises a DNA intercalating agent. In some cases, the plurality of nucleic acid binding moieties comprises psoralen. In some cases, the dendrimer further comprises an affinity tag. In some cases, the affinity tag comprises biotin. In some cases, the plurality of nucleic acid fragments further comprises an adaptor. In some cases, the plurality of nucleic acid fragments are fragments of chromosomal deoxyribonucleic acid (DNA). In some cases, the plurality of nucleic acid fragments further comprise barcodes. In some cases, the barcodes comprise dibenzocyclooctyl (DBCO) modified nucleotides. In some cases, the DBCO modified nucleotides are linked to a biotin azide. In some cases, the DBCO modified nucleotides are linked to a biotin azide via a photocleavable linkage. In some cases, the composition further comprises a streptavidin bead. In some cases, the composition further comprises an endonuclease. In some cases, the composition further comprises a ligase.

In another aspect, there are provided methods of nucleic acid processing, comprising: obtaining a stabilized sample comprising a nucleic acid molecule complexed to at least one nucleic acid binding protein; contacting the stabilized sample to a dendrimer comprising a plurality of nucleic acid binding moieties such that the nucleic acid molecule forms a complex with the plurality of nucleic acid binding moieties; contacting the complex to an endonuclease to cleave the nucleic acid molecule between contact points of the nucleic acid molecule and the plurality of nucleic acid binding moieties creating a plurality of fragments of the nucleic acid molecule each complexed with a nucleic acid binding moiety of the plurality of the nucleic acid binding moieties; isolating the product using an agent that binds to the dendrimer; joining the plurality of fragments to each other to create a concatemer comprising each of the plurality of fragments of the nucleic acid molecule; and isolating the concatemer from the dendrimer. In some cases, the stabilized sample comprises cross-linked chromatin. In some cases, the stabilized sample comprises cross-linked nuclei. In some cases, the plurality of nucleic acid binding proteins comprises a histone, a transcription factor, or combination thereof. In some cases, the plurality of nucleic acid binding moieties comprises a nucleic acid intercalator. In some cases, the plurality of nucleic acid binding moieties comprises psoralen, chlormethine, cyclophosphamide, chlorambucil, uramustine, melphalan, bendamustinc, bis(2-chloroethyl)ethylamine, bis(2-chloroethyl)methylamine, tris(2-chloroethyl)amine, isofamide, carmustine, lomustine, streptozocin, busulfan, cisplatin, carboplatin, cicycloplatin, eptaplatin, lobaplatin, miriplatin, nedaplatin, oxaliplatin, picoplatin, satraplatin, triplatin tetranitrate, procarbazine, altretamine, dacarbazine, mitozolomide, temozolomide, mitomycin C, nitrous acid, formaldehyde, acetylaldehyde, doxorubicin, daunorubicin, epirubicin, or idarubicin. In some cases, the dendrimer comprises poly(amidoamine) (PAMAM). In some cases, the dendrimer comprises about 16 to about 512 nucleic acid binding moieties. In some cases, the endonuclease comprises DNase I, DNase II, micrococcal nuclease, a restriction endonuclease, or a combination thereof. In some cases, the nucleic acid molecule is a chromosome. In some cases, the dendrimer further comprises an affinity tag. In some cases, the affinity tag comprises biotin. In some cases, the agent binds to the affinity tag. In some cases, the method further comprises contacting the product to a plurality of oligonucleotides and joining each fragment of the plurality of fragments to one of the plurality of oligonucleotides. In some cases, the plurality of oligonucleotides comprise barcodes. In some cases, the plurality of oligonucleotides comprise dibenzocyclooctyl (DBCO) modified nucleotides. In some cases, the method further comprises forming a linkage between the DBCO to a biotin azide. In some cases, the linkage is photocleavable. In some cases, a streptavidin bead is used to isolate the concatemer from the dendrimer. In some cases, the stabilized sample comprises a single cell. In some cases, the stabilized sample comprises a formalin-fixed paraffin embedded (FFPE) sample. In some cases, the stabilized sample comprises cell-free nucleic acids. In some cases, the stabilized sample comprises a microbiome. In some cases, the method further comprises obtaining a sequence of the concatemer.

In another aspect, there are provided methods of determining long range phase information, the method comprising: obtaining a stabilized sample comprising a nucleic acid molecule complexed to at least one nucleic acid binding protein; contacting the stabilized sample to a dendrimer comprising a plurality of nucleic acid binding moieties such that the nucleic acid molecule forms a complex with the plurality of nucleic acid binding moieties; contacting the complex to an endonuclease to cleave the nucleic acid molecule between contact points of the nucleic acid molecule and the plurality of nucleic acid binding moieties creating a plurality of fragments of the nucleic acid molecule each complexed with a nucleic acid binding moiety of the plurality of the nucleic acid binding moieties; isolate the product using an agent that binds to the dendrimer; joining the plurality of fragments to each other to create a concatemer comprising each of the plurality of fragments of the nucleic acid molecule; isolating the concatemer from the dendrimer; and obtaining a sequence of the concatemer, wherein the sequence comprises the long range phase information. In some cases, the stabilized sample comprises cross-linked chromatin. In some cases, the stabilized sample comprises cross-linked nuclei. In some cases, the plurality of nucleic acid binding proteins comprises a histone, a transcription factor, or a combination thereof. In some cases, the plurality of nucleic acid binding moieties comprises a nucleic acid intercalator. In some cases, the plurality of nucleic acid binding moieties comprises psoralen, chlormethine, cyclophosphamide, chlorambucil, uramustine, melphalan, bendamustine, bis(2-chloroethyl)ethylamine, bis(2-chloroethyl)methylamine, tris(2-chloroethyl)amine, isofamide, carmustine, lomustine, streptozocin, busulfan, cisplatin, carboplatin, cicycloplatin, eptaplatin, lobaplatin, miriplatin, nedaplatin, oxaliplatin, picoplatin, satraplatin, triplatin tetranitrate, procarbazine, altretamine, dacarbazine, mitozolomide, temozolomide, mitomycin C, nitrous acid, formaldehyde, acetylaldehyde, doxorubicin, daunorubicin, epirubicin, or idarubicin. In some cases, the dendrimer comprises poly(amidoamine) (PAMAM). In some cases, the dendrimer comprises about 16 to about 512 nucleic acid binding moieties. In some cases, the endonuclease comprises DNase I, DNase II, micrococcal nuclease, a restriction endonuclease, or a combination thereof. In some cases, the nucleic acid molecule is a chromosome. In some cases, the dendrimer further comprises an affinity tag. In some cases, the affinity tag comprises biotin. In some cases, the agent binds to the affinity tag. In some cases, the method comprises contacting the product to a plurality of oligonucleotides and joining each fragment of the plurality of fragments to one of the plurality of oligonucleotides. In some cases, the plurality of oligonucleotides comprise barcodes. In some cases, the plurality of oligonucleotides comprise dibenzocyclooctyl (DBCO) modified nucleotides. In some cases, the method further comprises forming a linkage between the DBCO to a biotin azide. In some cases, the linkage is photocleavable. In some cases, a streptavidin bead is used to isolate the concatemer from the dendrimer. In some cases, the stabilized sample comprises a single cell. In some cases, the stabilized sample comprises a formalin-fixed paraffin embedded (FFPE) sample. In some cases, the stabilized sample comprises a microbiome.

In a further aspect, there are provided methods of nucleic acid processing, comprising: obtaining a stabilized sample comprising a nucleic acid molecule complexed to at least one nucleic acid binding protein; contacting the stabilized sample to an endonuclease to create fragments of the nucleic acid molecule; contacting the fragments to a plurality of dendrimers comprising a plurality of oligonucleotides, wherein each dendrimer of the plurality of dendrimers comprises a unique barcode sequence and a constant sequence; joining the each of the fragments to at least one of the plurality of oligonucleotides of the plurality of dendrimers to form a complex; isolating the complex using an agent that binds to the plurality of dendrimers; and isolating the fragments joined to the oligonucleotides from the dendrimer. In some cases, the stabilized sample comprises cross-linked chromatin. In some cases, the stabilized sample comprises cross-linked nuclei. In some cases, the nucleic acid binding protein comprises a histone, a transcription factor, or a combination thereof. In some cases, the plurality of dendrimers are coupled to a solid surface. In some cases, the solid surface is a bead. In some cases, the plurality of dendrimers comprises poly(amidoamine) (PAMAM). In some cases, each of the plurality of dendrimers comprises about 16 to about 512 nucleic acid binding moieties. In some cases, the endonuclease DNase I, DNase II, micrococcal nuclease, a restriction endonuclease, or a combination thereof. In some cases, the nucleic acid molecule is a chromosome. In some cases, the plurality of dendrimers further comprises an affinity tag. In some cases, the affinity tag comprises biotin. In some cases, the agent binds to the affinity tag. In some cases, the stabilized sample comprises a single cell. In some cases, the stabilized sample comprises a formalin-fixed paraffin embedded (FFPE) sample. In some cases, the stabilized sample comprises cell-free nucleic acids. In some cases, the stabilized sample comprises a microbiome. In some cases, the method further comprises obtaining plurality of sequence reads of the plurality of fragments joined to the oligonucleotides.

In another aspect, there are provided methods of single cell genomic analysis, the method comprising: contacting a stabilized nucleus comprising a plurality of nucleic acid molecules bound to at least one nucleic acid binding protein of the single cell an endonuclease to create a plurality of nucleic acid fragments; contacting the plurality of nucleic acid fragments to a dendrimer comprising a plurality of oligonucleotides to link the plurality of nucleic acid fragments to the plurality of oligonucleotides, wherein the plurality of oligonucleotides each comprise a barcode sequence and a constant sequence; isolating the dendrimer linked to the plurality of oligonucleotides linked to the plurality of nucleic acid fragments from the nucleus using an agent that binds to the dendrimer, and sequencing the plurality of oligonucleotides linked to the plurality of nucleic acid fragments. In some cases, the nucleic acid binding protein comprises a histone, a transcription factor, or a combination thereof. In some cases, the dendrimer is coupled to a solid surface. In some cases, the solid surface is a bead. In some cases, the dendrimer comprises poly(amidoamine) (PAMAM). In some cases, the dendrimer comprises about 16 to about 512 nucleic acid binding moieties. In some cases, the endonuclease comprises DNase I, DNase II, micrococcal nuclease, a restriction endonuclease, or a combination thereof. In some cases, the plurality of nucleic acid molecules are chromosomes. In some cases, the dendrimer further comprises an affinity tag. In some cases, the amity tag comprises biotin. In some cases, the agent binds to the affinity tag.

In a further aspect, there are provided methods of processing a sample comprising cell-free nucleic acids, the method comprising: contacting a sample comprising a plurality of cell-free nucleic acids to a dendrimer comprising a plurality of nucleic acid binding moieties such that the plurality of cell-free nucleic acids forms a complex with the plurality of nucleic acid binding moieties; isolating the complex using an agent that binds to the dendrimer; joining the plurality of fragments to each other to create a concatemer comprising each of the plurality of cell-free nucleic acids; and isolating the concatemer from the dendrimer. In some cases, the plurality of nucleic acid binding moieties comprises a nucleic acid intercalator. In some cases, the plurality of nucleic acid binding moieties comprises psoralen, chlormethine, cyclophosphamide, chlorambucil, uramustine, melphalan, bendamustine, bis(2-chloroethyl)ethylamine, bis(2-chloroethyl)methylamine, tris(2-chloroethyl)amine, isofamide, carmustine, lomustine, streptozocin, busulfan, cisplatin, carboplatin, cicycloplatin, eptaplatin, lobaplatin, miriplatin, nedaplatin, oxaliplatin, picoplatin, satraplatin, triplatin tetranitrate, procarbazine, altretamine, dacarbazine, mitozolomide, temozolomide, mitomycin C, nitrous acid, formaldehyde, acetylaldehyde, doxorubicin, daunorubicin, epirubicin, or idarubicin. In some cases, the dendrimer comprises poly(amidoamine) (PAMAM). In some cases, the dendrimer comprises about 16 to about 512 nucleic acid binding moieties. In some cases, the dendrimer further comprises an affinity tag. In some cases, the affinity tag comprises biotin. In some cases, the agent binds to the affinity tag. In some cases, the method further comprises contacting the product to a plurality of oligonucleotides and joining each fragment of the plurality of fragments to one of the plurality of oligonucleotides. In some cases, the plurality of oligonucleotides comprise barcodes. In some cases, the plurality of oligonucleotides comprise dibenzocyclooctyl (DBCO) modified nucleotides. In some cases, the method further comprises forming a linkage between the DBCO to a biotin azide. In some cases, the linkage is photocleavable. In some cases, a streptavidin bead is used to isolate the concatemer from the dendrimer. In some cases, the method further comprises obtaining a sequence of the concatemer.

In another aspect, there are provided methods of processing a metagenomic sample, the method comprising: obtaining a stabilized sample comprising a plurality of nucleic acid molecules from a plurality of organisms, wherein each nucleic acid molecule of the plurality is complexed to at least one nucleic acid binding protein; contacting the stabilized sample to a plurality of dendrimers each dendrimer comprising a plurality of nucleic acid binding moieties such that each of the plurality of nucleic acid molecules forms a complex with the plurality of nucleic acid binding moieties of at least one of the plurality of dendrimers resulting in a plurality of complexes; contacting the plurality of complexes to an endonuclease to cleave the plurality of nucleic acid molecules between contact points of the nucleic acid molecule and the plurality of nucleic acid binding moieties creating a plurality of fragments of each of the plurality of nucleic acid molecules each fragment of the plurality of fragments complexed with a nucleic acid binding moiety of the plurality of the nucleic acid binding moieties; isolating the product using an agent that binds to the dendrimer; joining the plurality of fragments of each complex to each other to create a concatemer comprising each of the plurality of fragments bound to the dendrimer; isolating plurality of concatemers from each of the plurality of dendrimers; and obtaining a plurality of sequences of each of the plurality of concatemers, wherein each sequence of the plurality of sequences comprises sequence information from an organism of the plurality of organisms. In some cases, the plurality of nucleic acid binding proteins comprises a histone, a transcription factor, or a combination thereof. In some cases, the plurality of nucleic acid binding moieties comprises a nucleic acid intercalator. In some cases, the plurality of nucleic acid binding moieties comprises psoralen, chlormethine, cyclophosphamide, chlorambucil, uramustine, melphalan, bendamustine, bis(2-chloroethyl)ethylamine, bis(2-chloroethyl)methylamine, tris(2-chloroethyl)amine, isofamide, carmustine, lomustine, streptozocin, busulfan, cisplatin, carboplatin, cicycloplatin, eptaplatin, lobaplatin, miriplatin, nedaplatin, oxaliplatin, picoplatin, satraplatin, triplatin tetranitrate, procarbazine, altretamine, dacarbazine, mitozolomide, temozolomide, mitomycin C, nitrous acid, formaldehyde, acetylaldehyde, doxorubicin, daunorubicin, epirubicin, or idarubicin. In some cases, the dendrimer comprises poly(amidoamine) (PAMAM). In some cases, the dendrimer comprises about 16 to about 512 nucleic acid binding moieties. In some cases, the endonuclease comprises DNase I, DNase II, micrococcal nuclease, a restriction endonuclease, or a combination thereof. In some cases, the plurality of nucleic acid molecules are chromosomes. In some cases, the dendrimer further comprises an affinity tag. In some cases, the affinity tag comprises biotin. In some cases, the agent binds to the affinity tag. In some cases, the method further comprises contacting the product to a plurality of oligonucleotides and joining each fragment of the plurality of fragments to one of the plurality of oligonucleotides. In some cases, the plurality of oligonucleotides comprise barcodes. In some cases, the plurality of oligonucleotides comprise dibenzocyclooctyl (DBCO) modified nucleotides. In some cases, the method further comprises forming a linkage between the DBCO to a biotin azide. In some cases, the linkage is photocleavable. In some cases, a streptavidin bead is used to isolate the concatemer from the dendrimer. In some cases, the stabilized sample comprises a microbiome. In some cases, the method further comprises obtaining a sequence of the concatemer.

In another aspect, there are provided methods of spatial genomic analysis, the methods comprising: contacting a stabilized biological sample comprising a plurality of nucleic acid molecules bound to a nucleic acid binding protein to an endonuclease to create a plurality of nucleic acid fragments in the stabilized biological sample, wherein the stabilized biological sample is attached to a surface; contacting a plurality of dendrimers, each of the plurality of dendrimers comprising a unique barcode, to the biological sample, wherein each of the plurality of dendrimers binds to a unique position on the surface; sequencing each unique barcode of the plurality of dendrimers bound to the unique position on the surface; obtaining location information for each of the plurality of dendrimers bound to the unique position on the surface; creating a plurality of complexes each comprising a linkage between the plurality of nucleic acid fragments to the plurality of dendrimers bound to the unique position on the surface; isolating the plurality of complexes; and obtaining sequence information of the plurality of nucleic acid fragments and the barcodes of the plurality of complexes. In some cases, the plurality of dendrimers are coupled to a bead. In some cases, the dendrimer comprises poly(amidoamine) (PAMAM). In some cases, the dendrimer comprises about 16 to about 512 nucleic acid binding moieties. In some cases, the endonuclease comprises DNase I, DNase II, micrococcal nuclease, a restriction endonuclease, or a combination thereof. In some cases, the dendrimer further comprises an affinity tag. In some cases, the affinity tag comprises biotin. In some cases, the agent binds to the affinity tag. In some cases, the stabilized biological sample comprises a formalin-fixed paraffin embedded (FFPE) sample. In some cases, the stabilized biological sample comprises a section of a tissue sample. In some cases, the stabilized biological sample comprises cultured cells. In some cases, the method further comprises obtaining plurality of sequence reads of the plurality of nucleic acid fragments. In some cases, each of the plurality of fragments are linked to the barcode of the complex. In some cases, the method further comprises obtaining a sequence of each of the plurality of fragments linked to the barcode.

In another aspect, there are provided methods comprising: (a) obtaining a stabilized biological sample comprising a nucleic acid molecule complexed to at least one nucleic acid binding protein; (b) contacting the nucleic acid molecule with a dendrimer to form a complex, wherein one or more polymers of the dendrimer comprise a terminal primary amine; (c) cleaving the nucleic acid molecule into a plurality of segments comprising at least a first segment and a second segment; and (d) attaching the first segment and the second segment of the plurality of segments at a junction. In some cases, the dendrimer is modified with a crosslinker. In some cases, the method further comprises, prior to (b) contacting the dendrimer with a crosslinker. In some cases, the crosslinking agent comprises an intercalating agent, an antibiotic, or a minor groove binding agent. In some cases, the crosslinker comprises psoralen, chlormethine, cyclophosphamide, chlorambucil, uramustine, melphalan, bendamustine, bis(2-chloroethyl)ethylamine, bis(2-chloroethyl)methylamine, tris(2-chloroethyl)amine, isofamide, carmustine, lomustine, streptozocin, busulfan, cisplatin, carboplatin, cicycloplatin, eptaplatin, lobaplatin, miriplatin, nedaplatin, oxaliplatin, picoplatin, satraplatin, triplatin tetranitrate, procarbazine, altretamine, dacarbazine, mitozolomide, temozolomide, mitomycin C, nitrous acid, formaldehyde, acetylaldehyde, doxorubicin, daunorubicin, epirubicin, or idarubicin. In some cases, the psoralen comprises an N-hydroxysuccinimide (NHS) ester-conjugated psoralen. In some cases, the dendrimer comprises a polyamidoamine (PAMAM) dendrimer. In some cases, the method further comprises: (e) uncoupling the crosslinker from the dendrimer. In some cases, the uncoupling comprises a hot alkali treatment. In some cases, the uncoupling comprises exposure to UV radiation. In some cases, a portion of the plurality of segments are joined to form concatemers. In some cases, the concatemers comprise at least three segments. In some cases, the concatemers comprise at least four segments. In some cases, the concatemers comprise at least five segments. In some cases, the concatemers comprise at least six segments. In some cases, the concatemers comprise at least eight segments. In some cases, the concatemers comprise at least ten segments. In some cases, the dendrimer has a molecular weight of from 5 kilodaltons (kDa) to 125 kDa. In some cases, the dendrimer has a molecular weight of from 6 kDa to 8 kDa. In some cases, the dendrimer has a molecular weight of from 25 kDa to 35 kDa. In some cases, the dendrimer has a molecular weight of from 110 kDa to 125 kDa. In some cases, the dendrimer comprises from 32 to 512 reactive groups. In some cases, the dendrimer comprises about 32 reactive groups. In some cases, the dendrimer comprises about 128 reactive groups. In some cases, the dendrimer comprises about 512 reactive groups. In some cases, the method further comprises, subsequent to (b), photoactivating the dendrimer complex. In some cases, the method further comprises (f) subjecting the plurality of segments to size selection to obtain a plurality of selected segments. In some cases, the cleaving comprises contacting the nucleic acid molecule with a deoxyribonuclease (DNase). In some cases, the DNase comprises DNase I, DNase II, micrococcal nuclease, a restriction endonuclease, or a combination thereof. In some cases, the stabilized biological sample has been treated with a crosslinking agent. In some cases, the crosslinking agent is a chemical fixative. In some cases, the chemical fixative comprises formaldehyde, psoralen, disuccinimidyl glutarate (DSG), ethylene glycol bis(succinimidyl succinate) (EGS), ultraviolet light, or a combination thereof. In some cases, the crosslinking agent comprises chlormethine, cyclophosphamide, chlorambucil, uramustine, melphalan, bendamustine, bis(2-chloroethyl)ethylamine, bis(2-chloroethyl)methylamine, tris(2-chloroethyl)amine, isofamide, carmustine, lomustine, streptozocin, busulfan, cisplatin, carboplatin, cicycloplatin, eptaplatin, lobaplatin, miriplatin, nedaplatin, oxaliplatin, picoplatin, satraplatin, triplatin tetranitrate, procarbazine, altretamine, dacarbazine, mitozolomide, temozolomide, mitomycin C, nitrous acid, formaldehyde, acetylaldehyde, doxorubicin, daunorubicin, epirubicin, or idarubicin. In some cases, the crosslinking agent comprises an intercalating agent, an antibiotic, or a minor groove binding agent. In some cases, the stabilized biological sample is a crosslinked paraffin-embedded tissue sample. In some cases, the stabilized biological sample comprises a stabilized cell lysate. In some cases, the stabilized biological sample comprises a stabilized intact cell. In some cases, the stabilized biological sample comprises a stabilized intact nucleus. In some cases, step (c) is conducted prior to lysis of the intact cell or the intact nucleus. In some cases, the method further comprises, prior to step (d), lysing cells and/or nuclei in the stabilized biological sample. In some cases, the stabilized biological sample comprises fewer than 3,000,000 cells. In some cases, the stabilized biological sample comprises fewer than 1,000,000 cells. In some cases, the stabilized biological sample comprises fewer than 100,000 cells. In some cases, the attaching comprises filling in sticky ends using biotin tagged nucleotides and ligating blunt ends. In some cases, the attaching comprises contacting at least the first segment and the second segment to at least one bridge oligonucleotide. In some cases, the bridge oligonucleotide comprises a barcode sequence. In some cases, the attaching comprises contacting at least the first segment and the second segment to multiple bridge oligonucleotides in series. In some cases, the attaching results in samples, cells, nuclei, chromosomes, or nucleic acid molecules of the stabilized biological sample receiving a unique sequence of bridge oligonucleotides. In some cases, the attaching comprises contacting at least the first segment and the second segment to a barcode. In some cases, the method further comprises: (g) obtaining at least some sequence on each side of the junction to generate a first read pair. In some cases, the method further comprises: (h) mapping the first read pair to a set of contigs; and (i) determining a path through the set of contigs that represents an order and/or orientation to a genome. In some cases, the method further comprises: (h) mapping the first read pair to a set of contigs; and (i) determining, from the set of contigs, a presence of a structural variant or loss of heterozygosity in the stabilized biological sample. In some cases, the method further comprises: (h) mapping the first read pair to a set of contigs; and (i) assigning a variant in the set of contigs to a phase. In some cases, the method further comprises: (h) mapping the first read pair to a set of contigs; (i) determining, from the set of contigs, a presence of a variant in the set of contigs; and (j) conducting a step selected from one or more of: (1) identifying a disease stage, a prognosis, or a course of treatment for the stabilized biological sample; (2) selecting a drug based on the presence of the variant; or (3) identifying a drug efficacy for the stabilized biological sample.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

An understanding of the features and advantages of the present invention will be obtained by reference to the following detailed description that sets forth illustrative embodiments, in which the principles of the invention are utilized, and the accompanying drawings of which:

FIG. 1 shows a generation 2 PAMAM dendrimer.

FIG. 2 shows a dendrimer coupled with a DNA intercalating molecule (psoralen) and an affinity tag (biotin).

FIG. 3 shows a dendrimer-psoralen molecule complexed with chromatin.

FIG. 4 shows a dendrimer-psoralen molecule complexed with digested DNA that is to be ligated.

FIG. 5 shows a concatemer having an adaptor with a clickable moiety.

FIG. 6 shows results of processing a lambda DNA molecule using a dendrimer and digesting to create fragments that can be ligated.

FIG. 7 shows an exemplary Cu-Free click chemistry reaction.

FIG. 8 illustrates various components of an exemplary computer system according to various embodiments of the present disclosure.

FIG. 9 is a block diagram illustrating the architecture of an exemplary computer system that can be used in connection with various embodiments of the present disclosure.

FIG. 10 is a diagram illustrating an exemplary computer network that can be used in connection with various embodiments of the present disclosure.

FIG. 11 is a block diagram illustrating the architecture of another exemplary computer system that can be used in connection with various embodiments of the present disclosure.

### DETAILED DESCRIPTION

Provided herein are compositions, systems, and methods related to genomic analysis facilitated by dendrimer binding to nucleic acids in the sample. Methods herein can utilize dendrimers complexed to nucleic acid intercalating agents and/or oligonucleotides for binding sample nucleic acids that have been fragmented, for example with a nuclease. Dendrimers herein can also be complexed with an affinity agent, such as biotin, for purifying complexed dendrimers. Nucleic acid samples contemplated for use in compositions and methods herein include but are not limited to chromatin, digested chromatin, cell-free nucleic acids, single cells, nucleic acids from microbiome or environmental samples, and nucleic acids of a intact tissue section.

### Dendrimer Compositions

In an aspect, provided herein are compositions comprising, a dendrimer comprising a plurality of nucleic acid binding moieties. Compositions herein can further comprise a plurality of nucleic acid fragments. Nucleic acid fragments of the composition can be derived from a common chromosome. Alternatively, or in combination, nucleic acid fragments can be derived from different chromosomes. Nucleic acid fragments are sometimes found proximal to each other inside of a cell. In some cases, the nucleic acid fragments are cell-free nucleic acids. In some cases, the nucleic acid fragments are ribonucleic acids (RNA). In some cases, the nucleic acid fragments are deoxyribonucleic acid (DNA). In some cases, the nucleic acid fragments are double stranded. In some cases, the nucleic acid fragments are single stranded. Nucleic acid fragments of compositions herein can be crosslinked to other nuclear proteins, such as histones, transcription factors, and the like. Nucleic acid fragments of compositions herein can an adaptor. In some cases, nucleic acid fragments further comprise barcodes. In some cases, barcodes or adaptors herein comprise dibenzocyclooctyl (DBCO) modified nucleotides. In some cases, the DBCO modified nucleotides are linked to a biotin azide. In some cases, the DBCO modified nucleotides are linked to a biotin azide via a photocleavable linkage.

Dendrimers of compositions herein are comprised of any polymer suitable for binding nucleic acid molecules. Dendrimers can include but are not limited to poly(amidoamine) (PAMAM), poly(propylene imine) (PPI), triazine, citric acid, polyester, polyether, phosphorous, and peptide dendrimers. Dendrimers can also include moieties having special properties like fullerene, metals, mesogenic groups, and sterogenic molecules to develop fullerodendrimers, metallodendrimers, liquid crystalline dendrimers, and chiral dendrimers. Use of PEG, peptides, or amino acids and carbohydrate molecules as building groups or surface functionalities can lead to origination of PEGylated dendrimers, peptide dendrimers, and glycodendrimers. In some cases, dendrimers comprise poly(amidoamine) (PAMAM). The size of the dendrimers herein is determined by the type of application of the dendrimer. In some cases, a smaller dendrimer is needed. Alternatively, a larger dendrimer is suitable. In some cases, the dendrimer has a molecular weight of about 0.5 kDa to about 934 kDa. In some cases, the dendrimer has a molecular weight of from about 1.4 kDa to about 934 kDa, from about 3.2 kDa to about 934 kDa, from about 6.9 kDa to about 934 kDa, from about 14.2 kDa, to about 934 kDa, from about 28.8 kDa to about 934 kDa, from about 58 kDa to about 934 kDa, from about 116 kDa to about 934 kDa, from about 233 kDa to about 934 kDa, from about 467 kDa to about 934 kDa, from about 0.5 kDa to about 467 kDa, from about 1.4 kDa to about 467 kDa, from about 3.2 kDa to about 467 kDa, from about 6.9 kDa to about 467 kDa, from about 14.2 kDa to about 467 kDa, from about 28.8 kDa to about 467 kDa, from about 58 kDa to about 467 kDa, from about 116 kDa to about 467 kDa, from about 233 kDa to about 467 kDa, from about 0.5 kDa to about 233 kDa, from about 1.4 kDa to about 233 kDa, from about 3.2 kDa to about 233 kDa, from about 6.9 kDa to about 233 kDa, from about 14.2 kDa to about 233 kDa, from about 28.8 kDa to about 233 kDa, from about 58 kDa to about 233 kDa, from about 116 kDa to about 233 kDa, from about 0.5 kDa to about 116 kDa, from about 1.4 kDa to about 116 kDa, from about 3.2 kDa to about 116 kDa, from about 6.9 kDa to about 116 kDa, from about 14.2 kDa to about 116 kDa, from about 28.8 kDa to about 116 kDa, from about 58 kDa to about 116 kDa, from about 0.5 kDa to about 58 kDa, from about 1.4 kDa to about 116 kDa, from about 3.2 kDa to about 116 kDa, from about 6.9 kDa to about 116 kDa, from about 14.2 kDa to about 116 kDa, from about 14.2 kDa to about 116 kDa, from about 28.8 kDa to about 116 kDa, from about 58 kDa to about 116 kDa, from about 0.5 kDa to about 58 kDa, from about 1.4 kDa to about 58 kDa, from about 1.4 kDa to about 58 kDa, from about 3.2 kDa to about 58 kDa, from about 6.9 kDa to about 58 kDa, from about 6.9 kDa to about 58 kDa, from about 14.2 kDa to about 58 kDa, from about 28.8 kDa to about 58 kDa, from about 0.5 kDa to about 28.8 kDa, from about 1.4 kDa to about 28.8 kDa, from about 3.2 kDa to about 28.8 kDa, from about 6.9 kDa to about 28.8 kDa, from about 14.2 kDa to about 28.8 kDa, from about 0.5 kDa to about 14.2 kDa, from about 1.4 kDa to about 14.2 kDa, from about 3.2 kDa to about 14.2 kDa, from about 6.9 kDa to about 14.2 kDa, from about 0.5 kDa to about 6.9 kDa from about 1.4 kDa to about 6.9 kDa, from about 3.2 kDa to about 6.9 kDa, from about 0.5 kDa to about 3.2 kDa, from about 1.4 kDa to about 3.2 kDa, or about 0.5 kDa to about 1.4 kDa. In some cases, the dendrimer is about 3.2 kDa to about 116 kDa.

Dendrimers of compositions herein have a suitable number of binding moieties for a given application. For example, dendrimers herein can comprise about 4 to about 4096 nucleic acid binding moieties. In some cases, dendrimers herein comprise about 4 to about 4096, about 4 to about 2048, about 4 to about 1024, about 4 to about 512, about 4 to about 256, about 4 to about 128, about 4 to about 64, about 4 to about 32, about 4 to about 16, about 4 to about 8, about 8 to about 4096, about 8 to about 2048, about 8 to about 1024, about 8 to about 512, about 8 to about 256, about 8 to about 128, about 8 to about 64, about 8 to about 32, about 8 to about 16, about 16 to about 4096, about 16 to about 2048, about 16 to about 1024, about 16 to about 512, about 16 to about 256, about 16 to about 128, about 16 to about 64, about 16 to about 32, about 32 to about 4096, about 32 to about 2048, about 32 to about 1024, about 32 to about 512, about 32 to about 256, about 32 to about 128, about 32 to about 64, about 64 to about 4096, about 64 to about 2048, about 64 to about 1024, about 64 to about 512, about 64 to about 256, about 64 to about 128, about 128 to about 4096, about 128 to about 2048, about 128 to about 1024, about 128 to about 512, about 128 to about 256, about 256 to about 4096, about 256 to about 2048, about 256 to about 1024, about 256 to about 512, about 512 to about 4096, about 512 to about 2048, about 512 to about 1024, about 1024 to about 4096, about 1024 to about 2048, or about 2048 to about 4096 nucleic acid binding moieties.

Dendrimers herein can comprise a plurality of nucleic acid binding moieties. For example, nucleic acid binding moieties of dendrimers herein can comprise a plurality of DNA intercalating agents, a plurality of antibiotics, or a plurality of minor groove binding agents. In some cases, nucleic acid binding moieties of dendrimers herein comprise one or more of psoralen, chlormethine, cyclophosphamide, chlorambucil, uramustine, melphalan, bendamustine, bis(2-chloroethyl)ethylamine, bis(2-chloroethyl)methylamine, tris(2-chloroethyl)amine, isofamide, carmustine, lomustine, streptozocin, busulfan, cisplatin, carboplatin, cicycloplatin, eptaplatin, lobaplatin, miriplatin, nedaplatin, oxaliplatin, picoplatin, satraplatin, triplatin tetranitrate, procarbazine, altretamine, dacarbazine, mitozolomide, temozolomide, mitomycin C, nitrous acid, formaldehyde, acetylaldehyde, doxorubicin, daunorubicin, epirubicin, or idarubicin. In some cases, the nucleic acid binding moiety comprises psoralen. In some cases, dendrimers herein can further comprise an affinity tag for isolation of the dendrimers and bound nucleic acids from a solution. Affinity tags can comprise any antigen, receptor, or ligand that facilitates isolation without interfering with methods herein. In some cases, the affinity tag comprises biotin.

In additional aspects of compositions herein, in some cases compositions comprising dendrimers bound to nucleic acids further comprise a streptavidin bead. In some cases, compositions further comprise an endonuclease. In some cases, compositions further comprise a ligase.

### Methods of Proximity Ligation

In another aspect, provided herein are methods of nucleic acid processing. Methods of nucleic acid processing herein can comprise obtaining a stabilized sample comprising a nucleic acid molecule complexed to at least one nucleic acid binding protein. The method can also comprise contacting the stabilized sample to a dendrimer comprising a plurality of nucleic acid binding moieties such that the nucleic acid molecule forms a complex with the plurality of nucleic acid binding moieties. The method can further comprise contacting the complex to an endonuclease to cleave the nucleic acid molecule between contact points of the nucleic acid molecule and the plurality of nucleic acid binding moieties creating a plurality of fragments of the nucleic acid molecule each complexed with a nucleic acid binding moiety of the plurality of the nucleic acid binding moieties. Next, the method can comprise isolating the product using an agent that binds to the dendrimer. The method can also comprise joining the plurality of fragments to each other to create a concatemer comprising each of the plurality of fragments of the nucleic acid molecule. Then, the method can comprise isolating the concatemer from the dendrimer.

In aspects of methods herein, any suitable stabilized sample comprising nucleic acids, such as deoxyribonucleic acid (DNA) or ribonucleic acid (RNA), is contemplated for methods herein. For example, methods herein can use a stabilized sample comprising a plurality of cells, or a single cell. A stabilized sample can comprise a plurality of nuclei or a single nucleus. In some cases, the stabilized sample comprises a formalin-fixed paraffin embedded (FFPE) sample. In some cases, the stabilized sample comprises cell-free nucleic acids, such as cell-free DNA, cell-free RNA, circulating tumor DNA, circulating tumor RNA, or combination thereof. In some cases, the stabilized sample comprises cells, nuclei, or nucleic acids from a plurality of organisms, such as a microbiome sample, a forensic sample, or an environmental sample. In some cases, the sample comprises cross-linked chromatin. In some cases, the sample comprises cross-linked cell-free DNA. In some cases, the sample comprises cross-linked ribonucleoprotein complexes. In some cases, the sample comprises cross-linked nuclei. Samples of methods herein can further comprise one or more nucleic acid binding proteins, such as a histone, a transcription factor, or combination thereof.

In another aspect, methods herein utilize dendrimers that can comprise a plurality of nucleic acid binding moieties. For example, nucleic acid binding moieties of dendrimers herein can comprise a plurality of DNA intercalating agents, a plurality of antibiotics, or a plurality of minor groove binding agents. In some cases, nucleic acid binding moieties of dendrimers herein comprise one or more of psoralen, chlormethine, cyclophosphamide, chlorambucil, uramustine, melphalan, bendamustine, bis(2-chloroethyl)ethylamine, bis(2-chloroethyl)methylamine, tris(2-chloroethyl)amine, isofamide, carmustine, lomustine, streptozocin, busulfan, cisplatin, carboplatin, cicycloplatin, eptaplatin, lobaplatin, miriplatin, nedaplatin, oxaliplatin, picoplatin, satraplatin, triplatin tetranitrate, procarbazine, altretamine, dacarbazine, mitozolomide, temozolomide, mitomycin C, nitrous acid, formaldehyde, acetylaldehyde, doxorubicin, daunorubicin, epirubicin, or idarubicin. In some cases, the nucleic acid binding moiety comprises psoralen.

Dendrimers used in methods herein are comprised of any polymer suitable for binding nucleic acid molecules. In some cases, dendrimers comprise poly(amidoamine) (PAMAM). The size of the dendrimers herein is determined by the type of application of the dendrimer. In some cases, a smaller dendrimer is needed. Alternatively, a larger dendrimer is suitable. For example, dendrimers herein can comprise about 4 to about 4096 nucleic acid binding moieties. In some cases, dendrimers herein comprise about 4 to about 4096, about 4 to about 2048, about 4 to about 1024, about 4 to about 512, about 4 to about 256, about 4 to about 128, about 4 to about 64, about 4 to about 32, about 4 to about 16, about 4 to about 8, about 8 to about 4096, about 8 to about 2048, about 8 to about 1024, about 8 to about 512, about 8 to about 256, about 8 to about 128, about 8 to about 64, about 8 to about 32, about 8 to about 16, about 16 to about 4096, about 16 to about 2048, about 16 to about 1024, about 16 to about 512, about 16 to about 256, about 16 to about 128, about 16 to about 64, about 16 to about 32, about 32 to about 4096, about 32 to about 2048, about 32 to about 1024, about 32 to about 512, about 32 to about 256, about 32 to about 128, about 32 to about 64, about 64 to about 4096, about 64 to about 2048, about 64 to about 1024, about 64 to about 512, about 64 to about 256, about 64 to about 128, about 128 to about 4096, about 128 to about 2048, about 128 to about 1024, about 128 to about 512, about 128 to about 256, about 256 to about 4096, about 256 to about 2048, about 256 to about 1024, about 256 to about 512, about 512 to about 4096, about 512 to about 2048, about 512 to about 1024, about 1024 to about 4096, about 1024 to about 2048, or about 2048 to about 4096 nucleic acid binding moieties.

In some aspects of methods herein, nucleic acids are cleaved by a nuclease to yield nucleic acid fragments. Any suitable nuclease can be used in methods herein, for example a DNase, such as DNase I, DNase II, micrococcal nuclease, a restriction endonuclease, or a combination thereof.

In another aspect of methods herein, dendrimers herein can be isolated using an affinity tag to isolate the dendrimers and bound nucleic acids from a solution. Affinity tags can comprise any antigen, receptor, or ligand that facilitates isolation without interfering with methods herein. In some cases, the affinity tag comprises biotin. In methods herein, dendrimers and bound nucleic acids are isolated using an agent that binds to the affinity tag, such as streptavidin that binds to biotin.

In additional aspects of methods herein, the method can further comprise contacting the product of endonuclease cleavage to a plurality of oligonucleotides and joining each fragment of the plurality of fragments to at least one of the plurality of oligonucleotides. Oligonucleotides can comprise barcodes or unique molecular identifier sequence (UMI). Additionally, oligonucleotides can comprise one or more modified nucleotide. In some cases, the plurality of oligonucleotides comprise dibenzocyclooctyl (DBCO) modified nucleotides. DBCO modified nucleotides can facilitate linkage to other molecules, for example a linkage can be formed between DBCO and biotin azide. In some cases, the linkage is a photocleavable linkage.

In aspects of methods herein, dendrimers bound to nucleic acids can be isolated from a reaction mixture. Additionally, nucleic acids can be isolated from the dendrimer. This isolation can be accomplished using an agent that binds to the nucleic acid or to the dendrimer. In some cases as described herein, the dendrimer or the nucleic acid can comprise an affinity tag, a marker, or a label, such as biotin that can be bound by a binding agent, such as streptavidin. For example, a streptavidin bead can be used to isolate the concatemer from the dendrimer.

In additional aspects of methods herein, nucleic acid molecules bound to dendrimers are sequenced. For example, a concatemer of nucleic acid fragments from the stabilized sample are sequenced. Nucleic acid sequencing is contemplated to be done using any suitable sequencing method, such as those provided elsewhere herein, including but not limited to next-generation sequencing (e.g., Illumina, Nanoball sequencing and the like), long read sequencing (e.g., Oxford-Nanopore, Pacific Biosciences, and the like), or combinations thereof.

In another aspect, there are provided methods of determining long range phase information. Such methods herein can comprise obtaining a stabilized sample comprising a nucleic acid molecule complexed to at least one nucleic acid binding protein. Then, the stabilized sample can be contacted to a dendrimer comprising a plurality of nucleic acid binding moieties such that the nucleic acid molecule forms a complex with the plurality of nucleic acid binding moieties. The complex can then be contacted to an endonuclease to cleave the nucleic acid molecule between contact points of the nucleic acid molecule and the plurality of nucleic acid binding moieties creating a plurality of fragments of the nucleic acid molecule each complexed with a nucleic acid binding moiety of the plurality of the nucleic acid binding moieties. This complex can then be isolated using an agent that binds to the dendrimer. Then, the plurality of fragments of the complex can be joined to each other creating a concatemer that comprises each of the plurality of fragments of the nucleic acid molecule. The concatemer can be isolated from the dendrimer and a sequence of the concatemer can be obtained, thereby obtaining long range phase information.

In some aspects of methods herein, the complex of nucleic acid fragments complexed with the dendrimer is contacted to a plurality of oligonucleotides and joining each fragment of the plurality of fragments to one of the plurality of oligonucleotides. Oligonucleotides can comprise barcodes or unique molecular identifier sequence (UMI). Additionally, oligonucleotides can comprise one or more modified nucleotide. In some cases, the plurality of oligonucleotides comprise dibenzocyclooctyl (DBCO) modified nucleotides. DBCO modified nucleotides can facilitate linkage to other molecules, for example a linkage can be formed between DBCO and biotin azide. In some cases, the linkage is a photocleavable linkage.

### Methods of Single Cell Analysis

In another aspect, there are provided methods of single cell genomic analysis. Methods of single cell analysis herein can comprise contacting a stabilized nucleus comprising a plurality of nucleic acid molecules bound to at least one nucleic acid binding protein of the single cell an endonuclease to create a plurality of nucleic acid fragments. Next the plurality of nucleic acid fragments can be contacted to a dendrimer comprising a plurality of oligonucleotides to link the plurality of nucleic acid fragments to the plurality of oligonucleotides, wherein the plurality of oligonucleotides each comprise a barcode sequence and a constant sequence. The dendrimer linked to the plurality of oligonucleotides linked to the plurality of nucleic acid fragments from the nucleus can be isolated using an agent that binds to the dendrimer. Then the plurality of oligonucleotides linked to the plurality of nucleic acid fragments can be sequenced. In some cases, the nucleic acid binding protein comprises a histone, a transcription factor, or a combination thereof. In some cases, the plurality of nucleic acid molecules are chromosomes. Alternatively, the plurality of nucleic acid molecules are RNA.

In aspects of methods herein, the dendrimer can be coupled to a solid surface. In some cases, the dendrimer is coupled to a slide. In some cases, the dendrimer is coupled to a reaction vessel, such as a plate or a tube. In some cases, the dendrimer is coupled to a bead.

In another aspect, methods herein utilize dendrimers that can comprise a plurality of nucleic acid binding moieties. For example, nucleic acid binding moieties of dendrimers herein can comprise a plurality of DNA intercalating agents, a plurality of antibiotics, or a plurality of minor groove binding agents. In some cases, nucleic acid binding moieties of dendrimers herein comprise one or more of psoralen, chlormethine, cyclophosphamide, chlorambucil, uramustine, melphalan, bendamustine, bis(2-chloroethyl)ethylamine, bis(2-chloroethyl)methylamine, tris(2-chloroethyl)amine, isofamide, carmustine, lomustine, streptozocin, busulfan, cisplatin, carboplatin, cicycloplatin, eptaplatin, lobaplatin, miriplatin, nedaplatin, oxaliplatin, picoplatin, satraplatin, triplatin tetranitrate, procarbazine, altretamine, dacarbazine, mitozolomide, temozolomide, mitomycin C, nitrous acid, formaldehyde, acetylaldehyde, doxorubicin, daunorubicin, epirubicin, or idarubicin. In some cases, the nucleic acid binding moiety comprises psoralen.

Dendrimers useful in methods herein are comprised of any polymer suitable for binding nucleic acid molecules. In some cases, dendrimers comprise poly(amidoamine) (PAMAM). The size of the dendrimers herein is determined by the type of application of the dendrimer. In some cases, a smaller dendrimer is needed. Alternatively, a larger dendrimer is suitable. For example, dendrimers herein can comprise about 4 to about 4096 nucleic acid binding moieties. In some cases, dendrimers herein comprise about 4 to about 4096, about 4 to about 2048, about 4 to about 1024, about 4 to about 512, about 4 to about 256, about 4 to about 128, about 4 to about 64, about 4 to about 32, about 4 to about 16, about 4 to about 8, about 8 to about 4096, about 8 to about 2048, about 8 to about 1024, about 8 to about 512, about 8 to about 256, about 8 to about 128, about 8 to about 64, about 8 to about 32, about 8 to about 16, about 16 to about 4096, about 16 to about 2048, about 16 to about 1024, about 16 to about 512, about 16 to about 256, about 16 to about 128, about 16 to about 64, about 16 to about 32, about 32 to about 4096, about 32 to about 2048, about 32 to about 1024, about 32 to about 512, about 32 to about 256, about 32 to about 128, about 32 to about 64, about 64 to about 4096, about 64 to about 2048, about 64 to about 1024, about 64 to about 512, about 64 to about 256, about 64 to about 128, about 128 to about 4096, about 128 to about 2048, about 128 to about 1024, about 128 to about 512, about 128 to about 256, about 256 to about 4096, about 256 to about 2048, about 256 to about 1024, about 256 to about 512, about 512 to about 4096, about 512 to about 2048, about 512 to about 1024, about 1024 to about 4096, about 1024 to about 2048, or about 2048 to about 4096 nucleic acid binding moieties.

In some aspects of methods herein, nucleic acids are cleaved by a nuclease to yield nucleic acid fragments. Any suitable nuclease can be used in methods herein, for example a DNase, such as DNase I, DNase II, micrococcal nuclease, a restriction endonuclease, or a combination thereof.

In aspects of methods herein, dendrimers bound to nucleic acids can be isolated from a reaction mixture. Additionally, nucleic acids can be isolated from the dendrimer. This isolation can be accomplished using an agent that binds to the nucleic acid or to the dendrimer. In some cases as described herein, the dendrimer or the nucleic acid can comprise an affinity tag, a marker, or a label, such as biotin that can be bound by a binding agent, such as streptavidin. For example, a streptavidin bead can be used to isolate the concatemer from the dendrimer.

In additional aspects of methods herein, nucleic acid molecules from a single cell that are bound to dendrimers are sequenced. For example, a concatemer of nucleic acid fragments from the stabilized sample are sequenced. Nucleic acid sequencing is contemplated to be done using any suitable sequencing method, such as those provided elsewhere herein, including but not limited to next-generation sequencing (e.g., Illumina, Nanoball sequencing and the like), long read sequencing (e.g., Oxford-Nanopore. Pacific Biosciences, and the like), or combinations thereof.

### Methods of Cell-Free Nucleic Acid Analysis

In an aspect, there are provided, methods of processing a sample comprising cell-free nucleic acids. Such methods can comprise contacting a sample comprising a plurality of cell-free nucleic acids to a dendrimer comprising a plurality of nucleic acid binding moieties such that the plurality of cell-free nucleic acids forms a complex with the plurality of nucleic acid binding moieties. Next, the dendrimer-cell-free nucleic acid complex is isolated using an agent that binds to the dendrimer. The cell-free nucleic acids can be joined to each other to create a concatemer comprising each of the plurality of cell-free nucleic acids. Then this concatemer can be isolated from the dendrimer for further analysis and processing.

In another aspect, methods herein utilize dendrimers that can comprise a plurality of nucleic acid binding moieties. For example, nucleic acid binding moieties of dendrimers herein can comprise a plurality of DNA intercalating agents, a plurality of antibiotics, or a plurality of minor groove binding agents. In some cases, nucleic acid binding moieties of dendrimers herein comprise one or more of psoralen, chlormethine, cyclophosphamide, chlorambucil, uramustine, melphalan, bendamustine, bis(2-chloroethyl)ethylamine, bis(2-chloroethyl)methylamine, tris(2-chloroethyl)amine, isofamide, carmustine, lomustine, streptozocin, busulfan, cisplatin, carboplatin, cicycloplatin, eptaplatin, lobaplatin, miriplatin, nedaplatin, oxaliplatin, picoplatin, satraplatin, triplatin tetranitrate, procarbazine, altretamine, dacarbazine, mitozolomide, temozolomide, mitomycin C, nitrous acid, formaldehyde, acetylaldehyde, doxorubicin, daunorubicin, epirubicin, or idarubicin. In some cases, the nucleic acid binding moiety comprises psoralen.

Dendrimers useful in methods herein are comprised of any polymer suitable for binding nucleic acid molecules. In some cases, dendrimers comprise poly(amidoamine) (PAMAM). The size of the dendrimers herein is determined by the type of application of the dendrimer. In some cases, a smaller dendrimer is needed. Alternatively, a larger dendrimer is suitable. For example, dendrimers herein can comprise about 4 to about 4096 nucleic acid binding moieties. In some cases, dendrimers herein comprise about 4 to about 4096, about 4 to about 2048, about 4 to about 1024, about 4 to about 512, about 4 to about 256, about 4 to about 128, about 4 to about 64, about 4 to about 32, about 4 to about 16, about 4 to about 8, about 8 to about 4096, about 8 to about 2048, about 8 to about 1024, about 8 to about 512, about 8 to about 256, about 8 to about 128, about 8 to about 64, about 8 to about 32, about 8 to about 16, about 16 to about 4096, about 16 to about 2048, about 16 to about 1024, about 16 to about 512, about 16 to about 256, about 16 to about 128, about 16 to about 64, about 16 to about 32, about 32 to about 4096, about 32 to about 2048, about 32 to about 1024, about 32 to about 512, about 32 to about 256, about 32 to about 128, about 32 to about 64, about 64 to about 4096, about 64 to about 2048, about 64 to about 1024, about 64 to about 512, about 64 to about 256, about 64 to about 128, about 128 to about 4096, about 128 to about 2048, about 128 to about 1024, about 128 to about 512, about 128 to about 256, about 256 to about 4096, about 256 to about 2048, about 256 to about 1024, about 256 to about 512, about 512 to about 4096, about 512 to about 2048, about 512 to about 1024, about 1024 to about 4096, about 1024 to about 2048, or about 2048 to about 4096 nucleic acid binding moieties.

In aspects of methods herein, dendrimers bound to nucleic acids can be isolated from a reaction mixture. Additionally, nucleic acids can be isolated from the dendrimer. This isolation can be accomplished using an agent that binds to the nucleic acid or to the dendrimer. In some cases as described herein, the dendrimer or the nucleic acid can comprise an affinity tag, a marker, or a label, such as biotin that can be bound by a binding agent, such as streptavidin. For example, a streptavidin bead can be used to isolate the concatemer from the dendrimer.

In some aspects of methods herein, the cell-free nucleic acids complexed to the dendrimer can be contacted to a plurality of oligonucleotides and each cell-free nucleic acid of the plurality of cell-free nucleic acids are joined to one of the plurality of oligonucleotides. In some cases, the plurality of oligonucleotides comprise barcodes. In some cases, the plurality of oligonucleotides comprise dibenzocyclooctyl (DBCO) modified nucleotides. In some cases, the method further comprises forming a linkage between the DBCO and a biotin azide. In some cases, the linkage is photocleavable.

In additional aspects of methods herein, cell-free nucleic acid molecules bound to dendrimers are sequenced. For example, a concatemer of nucleic acid fragments from the stabilized sample are sequenced. Nucleic acid sequencing is contemplated to be done using any suitable sequencing method, such as those provided elsewhere herein, including but not limited to next-generation sequencing (e.g., Illumina. Nanoball sequencing and the like), long read sequencing (e.g., Oxford-Nanopore. Pacific Biosciences, and the like), or combinations thereof.

### Methods of Barcode Analysis

In another aspect, there are provided methods of nucleic acid processing comprising obtaining a stabilized sample comprising a nucleic acid molecule complexed to at least one nucleic acid binding protein. Then the stabilized sample can be contacted to an endonuclease to create fragments of the nucleic acid molecule. The fragments can then be contacted to a plurality of dendrimers comprising a plurality of oligonucleotides, wherein each dendrimer of the plurality of dendrimers comprises a unique barcode sequence and a constant sequence. Each of the fragments can then be joined to at least one of the plurality of oligonucleotides of the plurality of dendrimers to form a complex. This complex can be isolated using an agent that binds to the plurality of dendrimers. Then the fragments joined to the oligo nucleotides can be isolated from the dendrimer.

In aspects of methods herein, any suitable stabilized sample comprising nucleic acids, such as deoxyribonucleic acid (DNA) or ribonucleic acid (RNA), is contemplated for methods herein. For example, methods herein can use a stabilized sample comprising a plurality of cells, or a single cell. A stabilized sample can comprise a plurality of nuclei or a single nucleus. In some cases, the stabilized sample comprises a formalin-fixed paraffin embedded (FFPE) sample. In some cases, the stabilized sample comprises cell-free nucleic acids, such as cell-free DNA, cell-free RNA, circulating tumor DNA, circulating tumor RNA, or combination thereof. In some cases, the stabilized sample comprises cells, nuclei, or nucleic acids from a plurality of organisms, such as a microbiome sample, a forensic sample, or an environmental sample. In some cases, the sample comprises cross-linked chromatin. In some cases, the sample comprises cross-linked cell-free DNA. In some cases, the sample comprises cross-linked ribonucleoprotein complexes. In some cases, the sample comprises cross-linked nuclei. Samples of methods herein can further comprise one or more nucleic acid binding proteins, such as a histone, a transcription factor, or combination thereof.

In aspects of methods herein, the dendrimer can be coupled to a solid surface. In some cases, the dendrimer is coupled to a slide. In some cases, the dendrimer is coupled to a reaction vessel, such as a plate or a tube. In some cases, the dendrimer is coupled to a bead.

Dendrimers used in methods herein are comprised of any polymer suitable for binding nucleic acid molecules. In some cases, dendrimers comprise poly(amidoamine) (PAMAM). The size of the dendrimers herein is determined by the type of application of the dendrimer. In some cases, a smaller dendrimer is needed. Alternatively, a larger dendrimer is suitable. For example, dendrimers herein can comprise about 4 to about 4096 nucleic acid binding moieties. In some cases, dendrimers herein comprise about 4 to about 4096, about 4 to about 2048, about 4 to about 1024, about 4 to about 512, about 4 to about 256, about 4 to about 128, about 4 to about 64, about 4 to about 32, about 4 to about 16, about 4 to about 8, about 8 to about 4096, about 8 to about 2048, about 8 to about 1024, about 8 to about 512, about 8 to about 256, about 8 to about 128, about 8 to about 64, about 8 to about 32, about 8 to about 16, about 16 to about 4096, about 16 to about 2048, about 16 to about 1024, about 16 to about 512, about 16 to about 256, about 16 to about 128, about 16 to about 64, about 16 to about 32, about 32 to about 4096, about 32 to about 2048, about 32 to about 1024, about 32 to about 512, about 32 to about 256, about 32 to about 128, about 32 to about 64, about 64 to about 4096, about 64 to about 2048, about 64 to about 1024, about 64 to about 512, about 64 to about 256, about 64 to about 128, about 128 to about 4096, about 128 to about 2048, about 128 to about 1024, about 128 to about 512, about 128 to about 256, about 256 to about 4096, about 256 to about 2048, about 256 to about 1024, about 256 to about 512, about 512 to about 4096, about 512 to about 2048, about 512 to about 1024, about 1024 to about 4096, about 1024 to about 2048, or about 2048 to about 4096 nucleic acid binding moieties.

In some aspects of methods herein, nucleic acids are cleaved by a nuclease to yield nucleic acid fragments. Any suitable nuclease can be used in methods herein, for example a DNase, such as DNase I, DNase II, micrococcal nuclease, a restriction endonuclease, or a combination thereof.

In aspects of methods herein, dendrimers bound to nucleic acids can be isolated from a reaction mixture. Additionally, nucleic acids can be isolated from the dendrimer. This isolation can be accomplished using an agent that binds to the nucleic acid or to the dendrimer. In some cases as described herein, the dendrimer or the nucleic acid can comprise an affinity tag, a marker, or a label, such as biotin that can be bound by a binding agent, such as streptavidin. For example, a streptavidin bead can be used to isolate the concatemer from the dendrimer.

In additional aspects of methods herein, nucleic acid molecules from a single cell that are bound to dendrimers are sequenced. For example, a concatemer of nucleic acid fragments from the stabilized sample are sequenced. Nucleic acid sequencing is contemplated to be done using any suitable sequencing method, such as those provided elsewhere herein, including but not limited to next-generation sequencing (e.g., Illumina. Nanoball sequencing and the like), long read sequencing (e.g., Oxford-Nanopore, Pacific Biosciences, and the like), or combinations thereof.

### Methods of Microbiome Analysis

In another aspect, there are provided methods of processing a microbiome sample comprising obtaining a stabilized sample comprising a plurality of nucleic acid molecules from a plurality of organisms, wherein each nucleic acid molecule of the plurality is complexed to at least one nucleic acid binding protein. The stabilized sample can then be contacted to a plurality of dendrimers each dendrimer comprising a plurality of nucleic acid binding moieties such that each of the plurality of nucleic acid molecules forms a complex with the plurality of nucleic acid binding moieties of at least one of the plurality of dendrimers resulting in a plurality of complexes. The plurality of complexes can then be contacted to an endonuclease to cleave the plurality of nucleic acid molecules between contact points of the nucleic acid molecule and the plurality of nucleic acid binding moieties creating a plurality of fragments of each of the plurality of nucleic acid molecules each fragment of the plurality of fragments complexed with a nucleic acid binding moiety of the plurality of the nucleic acid binding moieties. Then the plurality of fragments complexed with nucleic acid binding moieties can be isolated using an agent that binds to the dendrimer. The plurality of fragments of each complex can be joined to each other to create a concatemer comprising each of the plurality of fragments bound to the dendrimer. The plurality of concatemers can be isolated from the dendrimers and the plurality of concatemers can be sequenced. The sequences obtained can comprise sequence information from an organism of the plurality of organisms. In some cases, the plurality of nucleic acid binding proteins comprises a histone, a transcription factor, or a combination thereof.

In another aspect, methods herein utilize dendrimers that can comprise a plurality of nucleic acid binding moieties. For example, nucleic acid binding moieties of dendrimers herein can comprise a plurality of DNA intercalating agents, a plurality of antibiotics, or a plurality of minor groove binding agents. In some cases, nucleic acid binding moieties of dendrimers herein comprise one or more of psoralen, chlormethine, cyclophosphamide, chlorambucil, uramustine, melphalan, bendamustine, bis(2-chloroethyl)ethylamine, bis(2-chloroethyl)methylamine, tris(2-chloroethyl)amine, isofamide, carmustine, lomustine, streptozocin, busulfan, cisplatin, carboplatin, cicycloplatin, eptaplatin, lobaplatin, miriplatin, nedaplatin, oxaliplatin, picoplatin, satraplatin, triplatin tetranitrate, procarbazine, altretamine, dacarbazine, mitozolomide, temozolomide, mitomycin C, nitrous acid, formaldehyde, acetylaldehyde, doxorubicin, daunorubicin, epirubicin, or idarubicin. In some cases, the nucleic acid binding moiety comprises psoralen.

Dendrimers used in methods herein are comprised of any polymer suitable for binding nucleic acid molecules. In some cases, dendrimers comprise poly(amidoamine) (PAMAM). The size of the dendrimers herein is determined by the type of application of the dendrimer. In some cases, a smaller dendrimer is needed. Alternatively, a larger dendrimer is suitable. For example, dendrimers herein can comprise about 4 to about 4096 nucleic acid binding moieties. In some cases, dendrimers herein comprise about 4 to about 4096, about 4 to about 2048, about 4 to about 1024, about 4 to about 512, about 4 to about 256, about 4 to about 128, about 4 to about 64, about 4 to about 32, about 4 to about 16, about 4 to about 8, about 8 to about 4096, about 8 to about 2048, about 8 to about 1024, about 8 to about 512, about 8 to about 256, about 8 to about 128, about 8 to about 64, about 8 to about 32, about 8 to about 16, about 16 to about 4096, about 16 to about 2048, about 16 to about 1024, about 16 to about 512, about 16 to about 256, about 16 to about 128, about 16 to about 64, about 16 to about 32, about 32 to about 4096, about 32 to about 2048, about 32 to about 1024, about 32 to about 512, about 32 to about 256, about 32 to about 128, about 32 to about 64, about 64 to about 4096, about 64 to about 2048, about 64 to about 1024, about 64 to about 512, about 64 to about 256, about 64 to about 128, about 128 to about 4096, about 128 to about 2048, about 128 to about 1024, about 128 to about 512, about 128 to about 256, about 256 to about 4096, about 256 to about 2048, about 256 to about 1024, about 256 to about 512, about 512 to about 4096, about 512 to about 2048, about 512 to about 1024, about 1024 to about 4096, about 1024 to about 2048, or about 2048 to about 4096 nucleic acid binding moieties.

In some aspects of methods herein, nucleic acids are cleaved by a nuclease to yield nucleic acid fragments. Any suitable nuclease can be used in methods herein, for example a DNase, such as DNase I, DNase II, micrococcal nuclease, a restriction endonuclease, or a combination thereof.

In aspects of methods herein, dendrimers bound to nucleic acids can be isolated from a reaction mixture. Additionally, nucleic acids can be isolated from the dendrimer. This isolation can be accomplished using an agent that binds to the nucleic acid or to the dendrimer. In some cases as described herein, the dendrimer or the nucleic acid can comprise an affinity tag, a marker, or a label, such as biotin that can be bound by a binding agent, such as streptavidin. For example, a streptavidin bead can be used to isolate the concatemer from the dendrimer.

In an aspect, methods herein can further comprise contacting the plurality of nucleic acid fragments complexed to the concatemer to a plurality of oligonucleotides and joining each fragment of the plurality of fragments to one of the plurality of oligonucleotides.

In some aspects of methods herein, the cell-free nucleic acids complexed to the dendrimer can be contacted to a plurality of oligonucleotides and each cell-free nucleic acid of the plurality of cell-free nucleic acids are joined to one of the plurality of oligonucleotides. In some cases, the plurality of oligonucleotides comprise barcodes. In some cases, the plurality of oligonucleotides comprise dibenzocyclooctyl (DBCO) modified nucleotides. In some cases, the method further comprises forming a linkage between the DBCO and a biotin azide. In some cases, the linkage is photocleavable.

In additional aspects of methods herein, nucleic acid molecules from a single cell that are bound to dendrimers are sequenced. For example, a concatemer of nucleic acid fragments from the stabilized sample are sequenced. Nucleic acid sequencing is contemplated to be done using any suitable sequencing method, such as those provided elsewhere herein, including but not limited to next-generation sequencing (e.g., Illumina, Nanoball sequencing and the like), long read sequencing (e.g., Oxford-Nanopore, Pacific Biosciences, and the like), or combinations thereof.

### Methods of Spatial Genomics

In another aspect, provided herein are methods of spatial genomic analysis comprising contacting a stabilized biological sample comprising a plurality of nucleic acid molecules bound to a nucleic acid binding protein to an endonuclease to create a plurality of nucleic acid fragments in the stabilized biological sample, wherein the stabilized biological sample is attached to a surface. Then a plurality of dendrimers, each of the plurality of dendrimers comprising a unique barcode, can be contacted to the biological sample, wherein each of the plurality of dendrimers binds to a unique position on the surface. Next, sequence is obtained from each unique barcode of the plurality of dendrimers bound to the unique position on the surface. Location information is then obtained for each of the plurality of dendrimers bound to the unique position on the surface. A plurality of complexes can be made each comprising a linkage between the plurality of nucleic acid fragments to the plurality of dendrimers bound to the unique position on the surface. Then the plurality of complexes can be isolated and sequence information of the plurality of nucleic acid fragments and the barcodes of the plurality of complexes can be obtained. In some cases, the plurality of dendrimers are coupled to beads.

Dendrimers used in methods herein are comprised of any polymer suitable for binding nucleic acid molecules. In some cases, dendrimers comprise poly(amidoamine) (PAMAM). The size of the dendrimers herein is determined by the type of application of the dendrimer. In some cases, a smaller dendrimer is needed. Alternatively, a larger dendrimer is suitable. For example, dendrimers herein can comprise about 4 to about 4096 nucleic acid binding moieties. In some cases, dendrimers herein comprise about 4 to about 4096, about 4 to about 2048, about 4 to about 1024, about 4 to about 512, about 4 to about 256, about 4 to about 128, about 4 to about 64, about 4 to about 32, about 4 to about 16, about 4 to about 8, about 8 to about 4096, about 8 to about 2048, about 8 to about 1024, about 8 to about 512, about 8 to about 256, about 8 to about 128, about 8 to about 64, about 8 to about 32, about 8 to about 16, about 16 to about 4096, about 16 to about 2048, about 16 to about 1024, about 16 to about 512, about 16 to about 256, about 16 to about 128, about 16 to about 64, about 16 to about 32, about 32 to about 4096, about 32 to about 2048, about 32 to about 1024, about 32 to about 512, about 32 to about 256, about 32 to about 128, about 32 to about 64, about 64 to about 4096, about 64 to about 2048, about 64 to about 1024, about 64 to about 512, about 64 to about 256, about 64 to about 128, about 128 to about 4096, about 128 to about 2048, about 128 to about 1024, about 128 to about 512, about 128 to about 256, about 256 to about 4096, about 256 to about 2048, about 256 to about 1024, about 256 to about 512, about 512 to about 4096, about 512 to about 2048, about 512 to about 1024, about 1024 to about 4096, about 1024 to about 2048, or about 2048 to about 4096 nucleic acid binding moieties.

In some aspects of methods herein, nucleic acids are cleaved by a nuclease to yield nucleic acid fragments. Any suitable nuclease can be used in methods herein, for example a DNase, such as DNase I. DNase II, micrococcal nuclease, a restriction endonuclease, or a combination thereof.

In aspects of methods herein, dendrimers bound to nucleic acids can be isolated from a reaction mixture. Additionally, nucleic acids can be isolated from the dendrimer. This isolation can be accomplished using an agent that binds to the nucleic acid or to the dendrimer. In some cases as described herein, the dendrimer or the nucleic acid can comprise an affinity tag, a marker, or a label, such as biotin that can be bound by a binding agent, such as streptavidin. For example, a streptavidin bead can be used to isolate the concatemer from the dendrimer.

In aspects of spatial genomics provided herein, the stabilized sample is contemplated to comprise any sample where the location of individual cells is important to analysis. For example, in some cases the stabilized biological sample comprises a formalin-fixed paraffin embedded (FFPE) sample. Alternatively or in combination, the stabilized biological sample comprises a section of a tissue sample. Alternatively or in combination, the stabilized biological sample comprises a section of a tumor sample. In some cases, the stabilized biological sample comprises cultured cells.

In additional aspects of methods herein, nucleic acid molecules from a single cell that are bound to dendrimers are sequenced. For example, a concatemer of nucleic acid fragments from the stabilized sample are sequenced. Nucleic acid sequencing is contemplated to be done using any suitable sequencing method, such as those provided elsewhere herein, including but not limited to next-generation sequencing (e.g., Illumina. Nanoball sequencing and the like), long read sequencing (e.g., Oxford-Nanopore, Pacific Biosciences, and the like), or combinations thereof.

In some aspects of methods herein, the cell-free nucleic acids complexed to the dendrimer can be contacted to a plurality of oligonucleotides and each cell-free nucleic acid of the plurality of cell-free nucleic acids are joined to one of the plurality of oligonucleotides. In some cases, the plurality of oligonucleotides comprise barcodes. In some cases, the plurality of oligonucleotides comprise dibenzocyclooctyl (DBCO) modified nucleotides. In some cases, the method further comprises forming a linkage between the DBCO and a biotin azide. In some cases, the linkage is photocleavable.

### Haplotype Phasing

In diploid genomes, it often important to know which allelic variants are linked on the same chromosome. This is known as the haplotype phasing. Short reads from high-throughput sequence data rarely allow one to directly observe which allelic variants are linked. Computational inference of haplotype phasing can be unreliable at long distances. The disclosure provides one or more methods that allow for determining which allelic variants are linked using allelic variants on read pairs. In some cases, phasing with methods of the present disclosure is conducted without imputation.

In various embodiments, the methods and compositions of the disclosure enable the haplotype phasing of diploid or polyploid genomes with regard to a plurality of allelic variants. The methods described herein can thus provide for the determination of linked allelic variants that are linked based on variant information from read pairs and/or assembled contigs using the same. Examples of allelic variants include, but are not limited to, those that are known from the 1000 genomes. UK10K, HapMap and other projects for discovering genetic variation among humans. Disease association to a specific gene can be revealed more easily by having haplotype phasing data as demonstrated, for example, by the finding of unlinked, inactivating mutations in both copies of SH3TC2 leading to Charcot-Marie-Tooth neuropathy (Lupski J R, Reid J G, Gonzaga-Jauregui C, et al. N. Engl. J. Med. 362:1181-91, 2010) and unlinked, inactivating mutations in both copies of ABCG5 leading to hypercholesterolemia 9 (Rios J, Stein E. Shendure J. et al. Hum. Mol. Genet. 19:4313-18.2010).

Humans are heterozygous at an average of 1 site in 1,000. In some cases, a single lane of data using high-throughput sequencing methods can generate at least about 150,000,000 read pairs. Read pairs can be about 100 base pairs long. From these parameters, one-tenth of all reads from a human sample is estimated to cover a heterozygous site. Thus, on average one-hundredth of all read pairs from a human sample is estimated to cover a pair of heterozygous sites. Accordingly, about 1,500,000 read pairs (one-hundredth of 150,000,000) provide phasing data using a single lane. With approximately 3 billion bases in the human genome, and one in one-thousand being heterozygous, there are approximately 3 million heterozygous sites in an average human genome. With about 1,500,000 read pairs that represent a pair of heterozygous sites, the average coverage of each heterozygous site to be phased using a single lane of a high-throughput sequence method is about (IX), using a typical high-throughput sequencing machine. A diploid human genome can therefore be reliably and completely phased with one lane of a high-throughput sequence data relating sequence variants from a sample that is prepared using the methods disclosed herein. In some examples, a lane of data can be a set of DNA sequence read data. In further examples, a lane of data can be a set of DNA sequence read data from a single run of a high-throughput sequencing instrument.

As the human genome consists of two homologous sets of chromosomes, understanding the true genetic makeup of an individual requires delineation of the maternal and paternal copies or haplotypes of the genetic material. Obtaining a haplotype in an individual is useful in several ways. First, haplotypes are useful clinically in predicting outcomes for donor-host matching in organ transplantation and are increasingly used as a means to detect disease associations. Second, in genes that show compound heterozygosity, haplotypes provide information as to whether two deleterious variants are located on the same allele, greatly affecting the prediction of whether inheritance of these variants is harmful. Third, haplotypes from groups of individuals have provided information on population structure and the evolutionary history of the human race. Lastly, recently described widespread allelic imbalances in gene expression suggest that genetic or epigenetic differences between alleles may contribute to quantitative differences in expression. An understanding of haplotype structure will delineate the mechanisms of variants that contribute to allelic imbalances.

In certain embodiments, the methods disclosed herein comprise an in vitro technique to fix and capture associations among distant regions of a genome as needed for long-range linkage and phasing. In some cases, the method comprises constructing and sequencing an concatemer library to deliver very genomically distant read pairs. In some cases, the interactions primarily arise from the random associations within a single DNA fragment. In some examples, the genomic distance between segments can be inferred because segments that are near to each other in a DNA molecule interact more often and with higher probability, while interactions between distant portions of the molecule will be less frequent. Consequently, there is a systematic relationship between the segments bound to a dendrimer that connecting two loci and their proximity on the input DNA. The disclosure can produce concatemers or dendrimer barcoded fragments capable of spanning the largest DNA fragments in an extraction. By applying improved assembly software tools that are specifically adapted to handle the type of data produced by the present method, a complete genomic assembly may be possible.

Extremely high phasing accuracy can be achieved by the data produced using the methods and compositions of the disclosure. In comparison to previous methods, the methods described herein can phase a higher proportion of the variants. Phasing can be achieved while maintaining high levels of accuracy. The techniques herein can allow for phasing at an accuracy of greater than about 70%, 80%, 90%, 95%, 96%, 97%, 98%, 99%, 99.9%, 99.99%, or 99.999%. The techniques herein can allow for accurate phasing with less than about 500× sequencing depth, 450× sequencing depth, 400× sequencing depth, 350× sequencing depth, 300× sequencing depth, 250× sequencing depth, 200× sequencing depth, 150× sequencing depth, 100× sequencing depth, or 50× sequencing depth. This phase information can be extended to longer ranges, for example, greater than about 200 kbp, about 300 kbp, about 400 kbp, about 500 kbp, about 600 kbp, about 700 kbp, about 800 kbp, about 900 kbp, about 1 Mbp, about 2 Mbp, about 3 Mbp, about 4 Mbp, about 5 Mbp. or about 10 Mbp. In some embodiments, more than 90% of the heterozygous SNPs for a human sample can be phased at an accuracy greater than 99% using less than about 250 million reads or read pairs, e.g., by using only 1 lane of Illumina HiSeq data. In other cases, more than about 40%, 50%, 60%, 70%, 80%, 90%, 95%, or 99% of the heterozygous SNPs for a human sample can be phased at an accuracy greater than about 70%, 80%, 90%, 95%, 96%, 97%, 98%, 99%, 99.9%, 99.99%, or 99.999% using less than about 250 million or about 500 million reads or read pairs, e.g., by using only 1 or 2 lanes of Illumina HiSeq data. For example, more than 95% or 99% of the heterozygous SNPs for a human sample can be phase at an accuracy greater than about 95% or 99% using less about 250 million or about 500 million reads. In further cases, additional variants can be captured by increasing the read length to about 200 bp, 250 bp, 300 bp, 350 bp, 400 bp, 450 bp, 500 bp, 600 bp, 800 bp, 1000 bp, 1500 bp, 2 kbp, 3 kbp, 4 kbp, 5 kbp, 10 kbp, 20 kbp, 50 kbp, or 100 kbp.

In other embodiments of the disclosure, the data from an concatemer or dendrimer barcoded fragment library can be used to confirm the phasing capabilities of the long-range read pairs. The accuracy of those results is on par with the best technologies previously available, but further extending to significantly longer distances. The current sample preparation protocol for a particular sequencing method recognizes variants located within a read-length. e.g., 150 bp, of a targeted site for phasing. In some cases, this proportion can be expanded to nearly all variable sites with the judicious choice of enzymes or with digestion conditions.

Haplotype phasing can include phasing the human leukocyte antigen (HLA) region (e.g., Class I HLA-A, B, and C; Class II HLA-DRB1/3/4/5, HLA-DQA1, HLA-DQB1, HLA-DPA1, HLA-DPB1). The HLA region of the genome is densely polymorphic and can be difficult to sequence or phase with standard sequencing approaches. Techniques of the present disclosure can provide for improved sequencing and phasing accuracy of the HLA region of the genome. Using techniques of the present disclosure, the HLA region of the genome can be phased accurately as part of phasing larger regions (e.g., chromosome arms, chromosomes, whole genomes) or on its own (e.g., by targeted enrichment such as hybrid capture). In an example, the HLA region on its own was phased accurately at a sequencing depth of approximately 300×. These techniques can provide advantages over traditional approaches for HLA analysis, such as long-range PCR; for example, long-range PCR can involve complex protocols and many separate reactions. As discussed further herein, samples can be multiplexed for sequencing analysis, for example by including sample-identifying barcodes in bridge oligonucleotides or elsewhere, and de-multiplexing the sequence information based on the barcodes. In an example, multiple samples are subjected to proximity ligation, barcoded with sample-identifying barcodes (e.g., in the bridge oligonucleotide), the HLA region is targeted (e.g., by hybrid capture), and multiplexed sequencing is conducted, allowing phasing of the HLA region for multiple samples. In some cases, phasing the HLA region is conducted without imputation.

Haplotype phasing can include phasing the killer cell immunoglobulin-like receptor (KIR) region. The KIR region of the genome is highly homologous and structurally dynamic due to transposon-mediated recombination, and can be difficult to sequence or phase with standard sequencing approaches. Techniques of the present disclosure can provide for improved sequencing and phasing accuracy of the KIR region of the genome. Using techniques of the present disclosure, the KIR region of the genome can be phased accurately as part of phasing larger regions (e.g., chromosome arms, chromosomes, whole genomes) or on its own (e.g., by targeted enrichment such as hybrid capture). These techniques can provide advantages over traditional approaches for HLA analysis, such as long-range PCR; for example, long-range PCR can involve complex protocols and many separate reactions. As discussed further herein, samples can be multiplexed for sequencing analysis, for example by including sample-identifying barcodes in bridge oligonucleotides or elsewhere, and de-multiplexing the sequence information based on the barcodes. In an example, multiple samples are subjected to proximity ligation, barcoded with sample-identifying barcodes (e.g., in the bridge oligonucleotide), the KIR region is targeted (e.g., by hybrid capture), and multiplexed sequencing is conducted, allowing phasing of the KIR region for multiple samples. At least about 8, 9, 10, 11, 12, 13, 14, 15, 16, 17, or more genes and/or pseudogenes can be phased. In some cases, phasing the KIR region is conducted without imputation.

### Metagenomics Analysis

In some embodiments, the compositions and methods described herein allow for the investigation of meta-genomes, for example, those found in the human gut or environmental samples. Accordingly, the partial or whole genomic sequences of some or all organisms that inhabit a given ecological environment can be investigated. Examples include random sequencing of all gut microbes, the microbes found on certain areas of skin, and the microbes that live in toxic waste sites. The composition of the microbe population in these environments can be determined using the compositions and methods described herein and as well as the aspects of interrelated biochemistries encoded by their respective genomes. The methods described herein can enable metagenomic studies from complex biological environments, for example, those that comprise more than 2, 3, 4, 5, 6, 7, 8, 9, 10, 12, 15, 20, 25, 30, 40, 50, 60, 70, 80, 90, 100, 125, 150, 175, 200, 250, 300, 400, 500, 600, 700, 800, 900, 1000, 5000, 10000 or more organisms and/or variants of organisms.

High degrees of accuracy required by cancer genome sequencing can be achieved using the methods and systems described herein. Inaccurate reference genomes can make base-calling challenges when sequencing cancer genomes. Heterogeneous samples and small starting materials, for example, a sample obtained by biopsy introduce additional challenges. Further, detection of large-scale structural variants and/or losses of heterozygosity is often crucial for cancer genome sequencing, as well as the ability to differentiate between somatic variants and errors in base-calling.

### Improved Sequencing Accuracy

Systems and methods described herein may generate accurate long sequences from complex samples containing 2, 3, 4, 5, 6, 7, 8, 9, 10, 12, 15, 20 or more varying genomes. Mixed samples of normal, benign, and/or tumor origin may be analyzed, optionally without the need for a normal control. In some embodiments, starting samples as little as 100 ng or even as little as hundreds of genome equivalents are utilized to generate accurate long sequences. Systems and methods described herein may allow for detection of large scale structural variants and rearrangements, Phased variant calls may be obtained over long sequences spanning about 1 kbp, about 2 kbp, about 5 kbp, about 10 kbp, about 20 kbp, about 50 kbp, about 100 kbp, about 200 kbp, about 500 kbp, about 1 Mbp, about 2 Mbp, about 5 Mbp, about 10 Mbp, about 20 Mbp, about 50 Mbp. or about 100 Mbp or more nucleotides. For example, phase variant call may be obtained over long sequences spanning about 1 Mbp or about 2 Mbp.

Haplotypes determined using the methods and systems described herein may be assigned to computational resources, for example, computational resources over a network, such as a cloud system. Short variant calls can be corrected, if necessary, using relevant information that is stored in the computational resources. Structural variants can be detected based on the combined information from short variant calls and the information stored in the computational resources. Problematic parts of the genome, such as segmental duplications, regions prone to structural variation, the highly variable and medically relevant MHC region, centromeric and telomeric regions, and other heterochromatic regions including, but not limited to, those with repeat regions, low sequence accuracy, high variant rates, ALU repeats, segmental duplications, or any other relevant problematic parts, can be reassembled for increased accuracy.

A sample type can be assigned to the sequence information either locally or in a networked computational resource, such as a cloud. In cases where the source of the information is known, for example, when the source of the information is from a cancer or normal tissue, the source can be assigned to the sample as part of a sample type. Other sample type examples generally include, but are not limited to, tissue type, sample collection method, presence of infection, type of infection, processing method, size of the sample, etc. In cases where a complete or partial comparison genome sequence is available, such as a normal genome in comparison to a cancer genome, the differences between the sample data and the comparison genome sequence can be determined and optionally output.

### Proximity Ligation to Create Concatemers

Provided herein are compositions, systems, and methods which allow concatemer formation using proximity ligation. For example, a biological sample, such as a stabilized biological sample having a nucleic acid molecule complexed to a nucleic acid binding protein, can be contacted with a dendrimer to form a complex. In another example, a biological sample is stabilized by being contacted with dendrimers to form a complex. Next, the nucleic acid molecule can be cleaved into a plurality of segments, for example at least a first segment and a second segment. Then, the plurality segments can be attached at a plurality of junctions, for example, the first segment and the second segment can be attached at a junction.

In certain aspects of methods herein, a biological sample, such as a stabilized biological sample having a nucleic acid molecule complexed with a nucleic acid binding protein and a dendrimer. In some cases, the dendrimer is conjugated with psoralen. In some cases, the dendrimer is conjugated with Azido-Peg4-N-hydroxysuccinimide (NHS) ester. In some cases, the NHS ester of the Azido-Peg4-NHS ester reacts with the primary amine on the dendrimer to result in a dendrimer having a reactive azide group. In some cases, carboxylated beads (e.g., magnetic beads) are prepared by conjugating using 1-ethyl-3-(3-dimethylaminopropyl)carbodiimide (EDC)/Sulpho-NHC chemistry with a dibenzocyclooctyne-amine (DBCO)-Peg4-amine building block. These prepared beads can be used to isolate the dendrimers, for example via magnetic separation methods prior to proximity ligation.

In some cases, the dendrimer is modified with a compound or contacted with a compound. For example, in some cases, the dendrimer is modified with psoralen. In some cases, the psoralen comprises an N-hydroxysuccinimide (NHS) ester-conjugated psoralen. In some cases, the dendrimer comprises a polyamidoamine (PAMAM) dendrimer. In some cases, the dendrimer is modified with a crosslinking agent such as, chlormethine, cyclophosphamide, chlorambucil, uramustine, melphalan, bendamustine, bis(2-chloroethyl)ethylamine, bis(2-chloroethyl)methylamine, tris(2-chloroethyl)amine, isofamide, carmustine, lomustine, streptozocin, busulfan, cisplatin, carboplatin, cicycloplatin, eptaplatin, lobaplatin, miriplatin, nedaplatin, oxaliplatin, picoplatin, satraplatin, triplatin tetranitrate, procarbazine, altretamine, dacarbazine, mitozolomide, temozolomide, mitomycin C, nitrous acid, formaldehyde, acetylaldehyde, doxorubicin, daunorubicin, epirubicin, or idarubicin. In some cases, the dendrimer is modified with an intercalating agent, an antibiotic, or a minor groove binding agent.

Methods herein can comprise uncoupling the compound from the dendrimer. For example, the compound, such as psoralen, can be uncoupled from the dendrimer using heat. In some cases, the compound, such as psoralen, is uncoupled from the dendrimer using alkali conditions or high pH. Alternatively, the compound, such as psoralen, is uncoupled from the dendrimer using heat and alkali conditions. The compound (e.g., psoralen) can also be uncoupled from the dendrimer using UV radiation.

Any suitable dendrimer can be used in methods herein. A dendrimer can have a molecular weight of about 5 kilodaltons (kDa) to about 125 kDa. In some cases, the dendrimer has a molecular weight of from 6 kDa to 8 kDa. In some cases, the dendrimer has a molecular weight of from 25 kDa to 35 kDa. In some cases, the dendrimer has a molecular weight of from 110 kDa to 125 kDa. In some cases, the dendrimer comprises from 32 to 512 reactive groups. In some cases, the dendrimer comprises about 32 reactive groups. In some cases, the dendrimer comprises about 128 reactive groups. In some cases, the dendrimer comprises about 512 reactive groups. In some cases, the dendrimer is a Gen3 dendrimer. In some cases, the dendrimer is a Gen5 dendrimer. In some cases, the dendrimer is a Gen7 dendrimer.

Methods herein can result in at least a portion of segments being joined into concatemers. For example, at least two segments, at least three segments, at least four segments, at least five segments, at least six segments, at least seven segments, at least eight segments, at least nine segments, at least ten segments, or more can be attached to form a concatemer. In some cases, an oligonucleotide is attached between each segment. In some cases, the oligonucleotide is a bridge oligonucleotide. In some cases, the oligonucleotide is an adapter oligonucleotide. In some cases, the oligonucleotide is a punctuation oligonucleotide. In some cases, the bridge oligonucleotide, the adapter oligonucleotide, and/or the punctuation oligonucleotide comprises a barcode sequence. In some cases, the bridge oligonucleotide, the adapter oligonucleotide, and/or the punctuation oligonucleotide is modified with a dibenzocyclooctyne (DBCO) moiety. In some cases, the DBCO moiety facilitates a copper free click chemistry. In some cases, a plurality of oligonucleotides are attached in series between each segment. The attaching can result in samples, cells, nuclei, chromosomes, or nucleic acid molecules of the stabilized biological sample receiving a unique sequence of oligonucleotides (e.g., bridge oligonucleotides).

In some cases, after the dendrimer is contacted with the stabilized biological sample to form a complex, the complex is photoactivated, for example by exposing the complex to UV radiation having a wavelength of about 360 nm, thereby creating a crosslinked complex. In some cases, the crosslinking is reversable without leaving an adduct on the nucleic acids.

Methods herein can further comprise subjecting the plurality of segments to size selection to obtain a plurality of selected segments. The size selection herein can include any suitable range of segment sizes.

Cleaving in methods provided herein can be done using any suitable method, for example by using a nuclease or a deoxyribonuclease (DNase). In some cases, the DNase comprises DNase I, DNaseII, micrococcal nuclease, a restriction endonuclease, or a combination thereof.

Stabilized biological samples in methods herein can be stabilized by being treated with a stabilizing agent or a crosslinking reagent. In some cases, the crosslinking agent is a chemical fixative, such as formaldehyde, psoralen, disuccinimidyl glutarate (DSG), ethylene glycol bis(succinimidyl succinate) (EGS), ultraviolet light, or a combination thereof. In some cases, the crosslinking agent comprises chlormethine, cyclophosphamide, chlorambucil, uramustine, melphalan, bendamustine, bis(2-chloroethyl)ethylamine, bis(2-chloroethyl)methylamine, tris(2-chloroethyl)amine, isofamide, carmustine, lomustine, streptozocin, busulfan, cisplatin, carboplatin, cicycloplatin, eptaplatin, lobaplatin, miriplatin, nedaplatin, oxaliplatin, picoplatin, satraplatin, triplatin tetranitrate, procarbazine, altretamine, dacarbazine, mitozolomide, temozolomide, mitomycin C, nitrous acid, formaldehyde, acetylaldehyde, doxorubicin, daunorubicin, epirubicin, or idarubicin. In some cases, the crosslinking agent comprises an intercalating agent, an antibiotic, or a minor groove binding agent. The stabilized biological sample can be a crosslinked paraffin-embedded tissue sample. In some cases, the stabilized biological sample comprises a stabilized intact cell or a stabilized intact nucleus. In some cases, the method comprises lysing cells and/or nuclei in the stabilized biological sample. The cleaving step of methods herein can be conducted prior to lysis of the intact cell or the intact nucleus.

Methods herein can be conducted on stabilized biological samples comprising small numbers of cells. For example, in some cases, the stabilized biological sample comprises fewer than about 3,000,000 cells. The stabilized biological sample can comprise fewer than about 1,000,000 cells, fewer than about 500,000 cells, fewer than about 400,000 cells, fewer than about 300,000 cells, fewer than about 200,000 cells, fewer than about 100,000 cells, or fewer.

In aspects of methods herein, the method can further comprise obtaining at least some sequence on each side of the junction to generate a first read pair. In addition, the method can further comprise mapping the first read pair to a set of contigs; and determining a path through the set of contigs that represents an order and/or orientation to a genome. Alternatively or in combination, the method can comprise mapping the first read pair to a set of contigs; and determining, from the set of contigs, a presence of a structural variant or loss of heterozygosity in the stabilized biological sample. Alternatively or in combination, the method can further comprise mapping the first read pair to a set of contigs; and assigning a variant in the set of contigs to a phase. Alternatively or in combination, the method can further comprise mapping the first read pair to a set of contigs; determining, from the set of contigs, a presence of a variant in the set of contigs; and conducting a step selected from one or more of: identifying a disease stage, a prognosis, or a course of treatment for the stabilized biological sample; selecting a drug based on the presence of the variant; or identifying a drug efficacy for the stabilized biological sample.

In aspects of methods herein, proximity ligation can be conducted with click chemistry, including copper-free click chemistry, such as with a DBCO modified bridge oligonucleotide attached between each segment of the concatemer. Then concatemers can be joined, for example via the dendrimers. To enrich for the ligated molecules, a feature of the bridge oligonucleotide can be targeted. In an example, a DBCO containing oligonucleotide can be reacted with an azide-biotin moiety which can be isolated with a streptavidin substrate, such as beads. In another example, a DBCO containing oligo nucleotide can be reacted with an azide-modified NHS—S—S-dPEG4-biotin which comprises a disulfide bond; azide can be added to the NHS—S—S-dPEG4-biotin using an azido-PEG3-amine, and in order to isolate the nucleic acids for library preparation, this disulfide bond can be reduced, for example by using DTT and heating, for example heating at 70° C. for about 10 minutes.

In aspects of methods herein, dendrimers with nucleic acid fragments contacted to them can be separated or isolated from the rest of the nucleic acids in the sample prior to proximity ligation of the nucleic acid fragments. This step can ensure that the concatemers formed by the proximity ligation comprise fragments that were contacted to the same dendrimer. This can mean that all the segments of a given concatemer were in proximity to each other in the original stabilized sample. Therefore, rather than just pairwise information about which nucleic acid regions were proximate to which other regions, such an approach can yield much more complex proximity information—e.g., that 3, 4, 5, 6, 7, 8, 9, 10, or more nucleic acid regions were all proximate to each other.

In some cases, dendrimers with nucleic acid fragments contacted to them can be separated or isolated from the rest of the nucleic acids to enable barcoding or tagging of those fragments, instead of proximity ligation. The fragments associated with a given dendrimer can be barcoded or tagged—for example, in a droplet or a well. After sequencing, sequences can be associated based on their barcodes and proximity information can be derived based on the barcodes, rather than from presence in the same concatemer as above. This proximity information can then be used as discussed herein. In one example, dendrimers are complexed to nucleic acids in a sample, thereby stabilizing them; the nucleic acids are then fragmented; dendrimers are then isolated with their complexed nucleic acid fragments and encapsulated in droplets; nucleic acids in droplets are labeled with a droplet-specific barcode or label; and nucleic acids are then sequenced, with barcode or label information used to associate fragments that were proximate to each other in the sample.

### Bridge Oligonucleotides

Methods provided herein can comprise attaching a first segment and a second segment of a plurality of segments at a junction. In some cases, attaching can comprise filling in sticky ends using biotin tagged nucleotides and ligating the blunt ends. In certain cases, attaching can comprise contacting at least the first segment and the second segment to a bridge oligonucleotide. In an exemplary workflow, a bridge oligonucleotide can be used to connect a first segment and a second segment where a nucleic acid is digested in situ to form the first segment and the second segment. The ends are polished and polyadenylated before ligating a bridge oligonucleotide to each of the first segment and the second segment. The first segment and the second segment are then ligated to create a junction comprising a bridge oligonucleotide. In various cases, attaching can comprise contacting at least the first segment and the second segment to a barcode.

In some embodiments, bridge oligonucleotides as provided herein can be from at least about 5 nucleotides in length to about 50 nucleotides in length. In certain embodiments, the bridge oligonucleotides can be from about 15 nucleotides in length to about 18 nucleotides in length. In various embodiments, the bridge oligonucleotides can be at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, or more nucleotides in length. In an example, the bridge oligonucleotides are at least 10 nucleotides in length. In another example, the bridge oligonucleotides are 12 nucleotides in length or about 12 nucleotides in length. In some cases, bridge oligonucleotides of at least 10 bp can increase stability and reduce adverse proximity ligation events, such as short inserts, interchromosomal ligations, non-specific ligations, and bridge self-ligations.

In some embodiments, the bridge oligonucleotides may comprise a barcode. In certain embodiments, the bridge oligonucleotides can comprise multiple barcodes (e.g., two or more barcodes). In various embodiments, the bridge oligonucleotides can comprise multiple bridge oligonucleotides coupled or connected together. In some embodiments, the bridge oligonucleotides may be coupled or linked to an immunoglobulin binding protein or fragment thereof, such as a Protein A, a Protein G, a Protein A/G, or a Protein L. In some cases, coupled bridge oligonucleotides may be delivered to a location in the sample nucleic acid where an antibody is bound.

A splitting and pooling approach can be employed to produce bridge oligonucleotides with unique barcodes. A population of samples can be split into multiple groups, bridge oligonucleotides can be attached to the samples such that the bridge oligonucleotide barcodes are different between groups but the same within a group, the groups of samples can be pooled together again, and this process can be repeated multiple times. For example, a population of polynucleotides can be split into Group A and Group B. First bridge oligonucleotides can be attached to the polynucleotides in Group A and second bridge oligonucleotides can be attached to the polynucleotides in Group B. Accordingly, the bridge oligonucleotide barcodes are the same within Group A, but the bridge oligonucleotides are different between Group A and Group B. Iterating this process can ultimately result in each sample in the population having a unique series of bridge oligonucleotide barcodes, allowing single-sample (e.g., single cell, single nucleus, single chromosome) analysis. In one illustrative example, a sample of crosslinked digested nuclei attached to a solid support of beads is split across 8 tubes, each containing 1 of 8 unique members of a first adaptor group (first iteration) comprising double-stranded DNA (dsDNA) adaptors to be ligated. Each of the 8 adaptors can have the same 5′ overhang sequence for ligation to the nucleic acid ends of the cross-linked chromatin aggregates in the nuclei, but otherwise has a unique dsDNA sequence. After the first adaptor group is ligated, the nuclei can be pooled back together and washed to remove the ligation reaction components. The scheme of distributing, ligating, and pooling can be repeated 2 additional times (2 iterations). Following ligation of members from each adaptor group, a cross-linked chromatin aggregate can be attached to multiple barcodes in series. In some cases, the sequential ligation of a plurality of members of a plurality of adaptor groups (iterations) results in barcode combinations. The number of barcode combinations available depends on the number of groups per iteration and the total number of barcode oligonucleotides used. For example, 3 iterations comprising 8 members each can have 83 possible combinations. In some cases, barcode combinations are unique. In some cases, barcode combinations are redundant. The total number of barcode combinations can be adjusted by increasing or decreasing the number of groups receiving unique barcodes and/or increasing or decreasing the number of iterations. When more than one adaptor group is used, a distributing, attaching, and pooling scheme can be used for iterative adaptor attachment. In some cases, the scheme of distributing, attaching, and pooling can be repeated at least 3, 4, 5, 6, 7, 8, 9, or 10 additional times. In some cases, the members of the last adaptor group include a sequence for subsequent enrichment of adaptor-attached DNA, for example, during sequencing library preparation through PCR amplification.

Iterating this process (of splitting and pooling) can ultimately result in each sample in the population having a unique series of bridge oligonucleotide barcodes, allowing single-sample (e.g., single cell, single nucleus, and single chromosome) analysis. An exemplary workflow includes using a splitting and pooling approach, where the nucleic acid is digested in situ and then end polished and polyadenylated. Single cells are dispensed, and a barcode is ligated to the ends present in each cell (e.g., barcode bc1). Cells are pooled and then single cells are isolated, and a second barcode is ligated to the ends present in each cell (e.g., barcode bc2). Cells are pooled again and separated into single cells before ligating a bridge adaptor (e.g., Bio-Bridge), which can be ligated to another DNA segment forming a junction between two segments having a unique combination of barcodes and adaptors identifying the cell from which the junction was derived (e.g., barcodes bc1 and bc2). The bridge adaptor can comprise one or more affinity reagents, such as biotin, for subsequent pull-down or other purification.

In another illustrative example, a sample of crosslinked digested nuclei attached to a solid support of beads can be split across eight tubes, each containing one of eight unique members of a first adaptor group (first iteration) comprising double-stranded DNA (dsDNA) adaptors to be ligated. Each of the eight adaptors can have the same 5′ overhang sequence for ligation to the nucleic acid ends of the cross-linked chromatin aggregates in the nuclei, but otherwise have a unique dsDNA sequence. After the first adaptor group is ligated, the nuclei can be pooled back together and washed to remove the ligation reaction components. The scheme of distributing, ligating, and pooling can be repeated two additional times (two iterations). Following ligation of members from each adaptor group, a cross-linked chromatin aggregate can be attached to multiple barcodes in series.

In some cases, the sequential ligation of a plurality of members of a plurality of adaptor groups (iterations) can result in barcode combinations. The number of barcode combinations available can depend on the number of groups per iteration and the total number of barcode oligonucleotides used. For example, three iterations comprising eight members each can have 83 possible combinations. In some cases, barcode combinations are unique. In certain cases, barcode combinations are redundant. The total number of barcode combinations can be adjusted by increasing or decreasing the number of groups receiving unique barcodes and/or increasing or decreasing the number of iterations. When more than one adaptor group is used, a distributing, attaching, and pooling scheme can be used for iterative adaptor attachment. In various cases, the scheme of distributing, attaching, and pooling can be repeated at least 3, 4, 5, 6, 7, 8, 9, 10, or more additional times. In some cases, the members of the last adaptor group may include a sequence for subsequent enrichment of adaptor-attached DNA, for example, during sequencing library preparation through PCR amplification.

In some cases, a three oligo design may be used, allowing for a split-pool strategy whereby two 96-well plates combined with eight different biotinylated oligos may be used, allowing for distinct barcoding of 73,728 different molecules. In certain cases, the first two sets of eight oligos are not biotinylated and the third set of eight oligos is biotinylated. In various cases, each barcoded oligonucleotide is directional allowing only one oligo to be added in each round. The bridge oligonucleotide can have a sequence that allows it to match up with a corresponding end.

In certain cases, the barcodes and adaptors may have a shorter sequence to reduce the amount of sequence space taken by the fully ligated bridges. In various cases, the bridge may take up 30 bp of sequence space. In some cases, the bridge may take up 54 bp of sequence space but offer additional positions for unique molecular identifiers (UMIs). In certain cases, UMIs may enable single-cell identification with 73,728 different combinations. In various cases, the first two oligo sets are unmodified and the third oligo set is biotinylated.

Barcode sequences in bridge adapters can be used to allow multiplexed sequencing of samples. For example, proximity ligation can be conducted on several different samples, with each sample using bridge oligonucleotides with different barcode sequences. The samples can then be pooled for multiplexed sequencing analysis, and sequence information can be de-multiplexed back to the individual samples based on the barcode sequences.

### Clinical Applications

The methods of the present disclosure can be used in the analysis of genetic information of selective genomic regions of interest as well as genomic regions which may interact with the selective region of interest. Amplification methods as disclosed herein can be used in devices, kits, and methods for genetic analysis, such as, but not limited to, those found in U.S. Pat. Nos. 6,449,562, 6,287,766, 7,361,468, 7,414,117, 6,225,109, and 6,110,709. In some cases, amplification methods of the present disclosure can be used to amplify target nucleic acid for DNA hybridization studies to determine the presence or absence of polymorphisms. The polymorphisms, or alleles, can be associated with diseases or conditions such as genetic disease. In other cases, the polymorphisms can be associated with susceptibility to diseases or conditions, for example, polymorphisms associated with addiction, degenerative and age-related conditions, cancer, and the like. In other cases, the polymorphisms can be associated with beneficial traits such as increased coronary health, or resistance to diseases such as HIV or malaria, or resistance to degenerative diseases such as osteoporosis. Alzheimer's, or dementia.

The compositions and methods of the disclosure can be used for diagnostic, prognostic, therapeutic, patient stratification, drug development, treatment selection, and screening purposes. The present disclosure provides the advantage that many different target molecules can be analyzed at one time from a single biomolecular sample using the methods of the disclosure. This allows, for example, for several diagnostic tests to be performed on one sample.

The composition and methods of the disclosure can be used in genomics. The methods described herein can provide an answer rapidly which is very desirable for this application. The methods and composition described herein can be used in the process of finding biomarkers that may be used for diagnostics or prognostics and as indicators of health and disease. The methods and composition described herein can be used to screen for drugs. e.g., drug development, selection of treatment, determination of treatment efficacy and/or identify targets for pharmaceutical development. The ability to test gene expression on screening assays involving drugs is very important because proteins are the final gene product in the body. In some embodiments, the methods and compositions described herein will measure both protein and gene expression simultaneously which will provide the most information regarding the particular screening being performed.

The composition and methods of the disclosure can be used in gene expression analysis. The methods described herein discriminate between nucleotide sequences. The difference between the target nucleotide sequences can be, for example, a single nucleic acid base difference, a nucleic acid deletion, a nucleic acid insertion, or rearrangement. Such sequence differences involving more than one base can also be detected. The process of the present disclosure is able to detect infectious diseases, genetic diseases, and cancer. It is also useful in environmental monitoring, forensics, and food science. Examples of genetic analyses that can be performed on nucleic acids include, e.g., SNP detection. STR detection, RNA expression analysis, promoter methylation, gene expression, virus detection, viral subtyping, and drug resistance.

The present methods can be applied to the analysis of biomolecular samples obtained or derived from a patient so as to determine whether a diseased cell type is present in the sample, the stage of the disease, the prognosis for the patient, the ability to the patient to respond to a particular treatment, or the best treatment for the patient. The present methods can also be applied to identify biomarkers for a particular disease.

In some embodiments, the methods described herein are used in the diagnosis of a condition. As used herein the term “diagnose” or “diagnosis” of a condition may include predicting or diagnosing the condition, determining predisposition to the condition, monitoring treatment of the condition, diagnosing a therapeutic response of the disease, or prognosis of the condition, condition progression, or response to particular treatment of the condition. For example, a blood sample can be assayed according to any of the methods described herein to determine the presence and/or quantity of markers of a disease or malignant cell type in the sample, thereby diagnosing or staging a disease or a cancer.

In some embodiments, the methods and composition described herein are used for the diagnosis and prognosis of a condition.

Numerous immunologic, proliferative, and malignant diseases and disorders are especially amenable to the methods described herein. Immunologic diseases and disorders include allergic diseases and disorders, disorders of immune function, and autoimmune diseases and conditions. Allergic diseases and disorders include, but are not limited to, allergic rhinitis, allergic conjunctivitis, allergic asthma, atopic eczema, atopic dermatitis, and food allergy. Immunodeficiencies include, but are not limited to, severe combined immunodeficiency (SCID), hypereosinophilic syndrome, chronic granulomatous disease, leukocyte adhesion deficiency I and II, hyper IgE syndrome. Chediak Higashi, neutrophilias, neutropenias, aplasias. Agammaglobulinemia, hyper-IgM syndromes, DiGeorge/Velocardial-facial syndromes and Interferon gamma-TH 1 pathway defects. Autoimmune and immune dysregulation disorders include, but are not limited to, rheumatoid arthritis, diabetes, systemic lupus erythematosus. Graves' disease, Graves ophthalmopathy. Crohn's disease, multiple sclerosis, psoriasis, systemic sclerosis, goiter and struma lymphomatosa (Hashimoto's thyroiditis, lymphadenoid goiter), alopecia aerata, autoimmune myocarditis, lichen sclerosis, autoimmune uveitis, Addison's disease, atrophic gastritis, myasthenia gravis, idiopathic thrombocytopenic purpura, hemolytic anemia, primary biliary cirrhosis, Wegener's granulomatosis, polyarteritis nodosa, and inflammatory bowel disease, allograft rejection and tissue destructive from allergic reactions to infectious microorganisms or to environmental antigens.

Proliferative diseases and disorders that may be evaluated by the methods of the disclosure include, but are not limited to, hemangiomatosis in newborns; secondary progressive multiple sclerosis; chronic progressive myelodegenerative disease; neurofibromatosis; ganglioneuromatosis; keloid formation; Paget's Disease of the bone; fibrocystic disease (e.g., of the breast or uterus); sarcoidosis; Peyronie's and Dupuytren's fibrosis, cirrhosis, atherosclerosis, and vascular restenosis.

Malignant diseases and disorders that may be evaluated by the methods of the disclosure include both hematologic malignancies and solid tumors.

Hematologic malignancies are especially amenable to the methods of the disclosure when the sample is a blood sample, because such malignancies involve changes in blood-borne cells. Such malignancies include non-Hodgkin's lymphoma, Hodgkin's lymphoma, non-B cell lymphomas, and other lymphomas, acute or chronic leukemias, polycythemias, thrombocythemias, multiple myeloma, myelodysplastic disorders, myeloproliferative disorders, myelofibroses, atypical immune lymphoproliferations and plasma cell disorders.

Plasma cell disorders that may be evaluated by the methods of the disclosure include multiple myeloma, amyloidosis and Waldenstrom's macroglobulinemia.

Example of solid tumors include, but are not limited to, colon cancer, breast cancer, lung cancer, prostate cancer, brain tumors, central nervous system tumors, bladder tumors, melanomas, liver cancer, osteosarcoma and other bone cancers, testicular and ovarian carcinomas, head and neck tumors, and cervical neoplasms.

Genetic diseases can also be detected by the process of the present disclosure. This can be carried out by prenatal or post-natal screening for chromosomal and genetic aberrations or for genetic diseases. Examples of detectable genetic diseases include: 21 hydroxylase deficiency, cystic fibrosis, Fragile X Syndrome. Turner Syndrome. Duchenne Muscular Dystrophy. Down Syndrome or other trisomies, heart disease, single gene diseases. HLA typing, phenylketonuria, sickle cell anemia. Tay-Sachs Disease, thalassemia, Klinefelter Syndrome. Huntington Disease, autoimmune diseases, lipidosis, obesity defects, hemophilia, inborn errors of metabolism, and diabetes.

Methods of the present disclosure can be used to detect genetic or genomic features associated with genetic diseases including, but not limited to, gene fusions, structural variants, rearrangements, and changes in topology such as missing or altered TAD boundaries, changes in TAD subtype, changes in compartment, changes in chromatin type, and changes in modification status such as methylation status (e.g., CpG methylation, H3K4me3, H3K27me3, or other histone methylation).

The methods described herein can be used to diagnose pathogen infections, for example, infections by intracellular bacteria and viruses, by determining the presence and/or quantity of markers of bacterium or virus, respectively, in the sample.

A wide variety of infectious diseases can be detected by the process of the present disclosure. The infectious diseases can be caused by bacterial, viral, parasite, and fungal infectious agents. The resistance of various infectious agents to drugs can also be determined using the present disclosure.

Bacterial infectious agents which can be detected by the present disclosure include Escherichia coli, Salmonella, Shigella, Klebstella, Pseudomonas, Listeria monocytogenes, Mycobacterium tuberculosis, Mycobacterium aviumintracellulare, Yersinia, Francisella, Pasteurella, Brucella, Clostridia, Bordetella pertussis, Bacteroides, Staphylococcus aureus, Streptococcus pneumonia, B-Hemolytic strep., Corynebacteria, Legionella, Mycoplasma, Ureaplasma, Chlamydia, Neisseria gonorrhea, Neisseria meningitides, Hemophilus influenza, Enterococcus faecalis, Proteus vulgaris, Proteus mirabilis, Helicobacter pylori, Treponema palladium. Borrelia burgdorferi, Borrelia recurrentis, Rickettsial pathogens, Nocardia, and Actinomycetes.

Fungal infectious agents which can be detected by the present disclosure include Cryptococcus neoformans, Blastomyces dermatitidis, Histoplasma capsulatum, Coccidioides immitis, Paracoccidioides brasiliensis, Candida albicans, Aspergillus fumigautus, Plycomycetes (Rhizopus), Sporothrix schenckii, Chromomycosis, and Maduromycosis.

Viral infectious agents which can be detected by the present disclosure include human immunodeficiency virus, human T-cell lymphocytotrophic virus, hepatitis viruses (e.g., Hepatitis B Virus and Hepatitis C Virus). Epstein-Barr virus, cy tomegalovirus, human papillomaviruses, orthomyxo viruses, paranyxo viruses, adenoviruses, corona viruses, rhabdo viruses, polio viruses, toga viruses, bunya viruses, arena viruses, rubella viruses, and reo viruses.

Parasitic agents which can be detected by the present disclosure include Plasmodium falciparum, Plasmodium malaria, Plasmodium vivax, Plasmodium ovale, Onchoverva volvuhrs, Leishmania, Trypanosoma spp., Schistosoma spp., Entamoeba histolytica, Cryptosporidium, Giardia spp., Trichimonas spp., Balatidium coli, Wuchereria bancrofti, Toxoplasma spp., Enterobius vermicularis, Ascaris lumbricoides, Trichuris trichiura, Dracunculus medinensis, Trematodes, Diphyllobothrium latum, Taenia spp., Pneumocystis carinii, and Necator americanis.

The present disclosure is also useful for detection of drug resistance by infectious agents. For example, vancomycin-resistant Enterococcus faecium, methicillin-resistant Staphylococcus aureus, penicillin-resistant Streptococcus pneunoniae, multi-drug resistant Mycobacterium tuberculosis, and AZT-resistant human immunodeficiency virus can all be identified with the present disclosure.

Thus, the target molecules detected using the compositions and methods of the disclosure can be either patient markers (such as a cancer marker) or markers of infection with a foreign agent, such as bacterial or viral markers.

The compositions and methods of the disclosure can be used to identify and/or quantify a target molecule whose abundance is indicative of a biological state or disease condition, for example, blood markers that are upregulated or downregulated as a result of a disease state.

In some embodiments, the methods and compositions of the present disclosure can be used for cytokine expression. The low sensitivity of the methods described herein would be helpful for early detection of cytokines, e.g., as biomarkers of a condition, diagnosis, or prognosis of a disease such as cancer, and the identification of subclinical conditions.

Methods of the present disclosure can be used to detect genetic or genomic features associated with cancer including, but not limited to, gene fusions, structural variants, rearrangements, and changes in topology such as missing or altered TAD boundaries, changes in TAD subtype, changes in compartment, changes in chromatin type, and changes in modification status such as methylation status (e.g., CpG methylation. H3K4me3. H3K27me3, or other histone methylation).

### Samples

The different samples from which the target polynucleotides are derived can comprise multiple samples from the same individual, samples from different individuals, or combinations thereof. In some embodiments, a sample comprises a plurality of polynucleotides from a single individual. In some embodiments, a sample comprises a plurality of polynucleotides from two or more individuals. An individual is any organism or portion thereof from which target polynucleotides can be derived, non-limiting examples of which include plants, animals, fungi, protists, monerans, viruses, mitochondria, and chloroplasts. Sample polynucleotides can be isolated from a subject, such as a cell sample, tissue sample, or organ sample derived therefrom, including, for example, cultured cell lines, biopsy, blood sample, or fluid sample containing a cell. The subject may be an animal including, but not limited to, an animal such as a cow, a pig, a mouse, a rat, a chicken, a cat, a dog, etc., and is usually a mammal, such as a human. Samples can also be artificially derived, such as by chemical synthesis. In some embodiments, the samples comprise DNA. In some embodiments, the samples comprise genomic DNA. In some embodiments, the samples comprise mitochondrial DNA, chloroplast DNA, plasmid DNA, bacterial artificial chromosomes, yeast artificial chromosomes, oligonucleotide tags, or combinations thereof. In some embodiments, the samples comprise DNA generated by primer extension reactions using any suitable combination of primers and a DNA polymerase including, but not limited to, polymerase chain reaction (PCR), reverse transcription, and combinations thereof. Where the template for the primer extension reaction is RNA, the product of reverse transcription is referred to as complementary DNA (cDNA). Primers useful in primer extension reactions can comprise sequences specific to one or more targets, random sequences, partially random sequences, and combinations thereof. Reaction conditions suitable for primer extension reactions are known. In general, sample polynucleotides comprise any polynucleotide present in a sample, which may or may not include target polynucleotides.

In some embodiments, nucleic acid template molecules (e.g., DNA or RNA) are isolated from a biological sample containing a variety of other components, such as proteins, lipids, and non-template nucleic acids. Nucleic acid template molecules can be obtained from any cellular material, obtained from an animal, plant, bacterium, fungus, or any other cellular organism. Biological samples for use in the present disclosure include viral particles or preparations. Nucleic acid template molecules can be obtained directly from an organism or from a biological sample obtained from an organism, e.g., from blood, urine, cerebrospinal fluid, seminal fluid, saliva, sputum, stool, and tissue. Any tissue or body fluid specimen may be used as a source for nucleic acid for use in the disclosure. Nucleic acid template molecules can also be isolated from cultured cells, such as a primary cell culture or a cell line. The cells or tissues from which template nucleic acids are obtained can be infected with a virus or other intracellular pathogen. A sample can also be total RNA extracted from a biological specimen, a cDNA library, viral, or genomic DNA. A sample may also be isolated DNA from a non-cellular origin, e.g., amplified/isolated DNA from the freezer.

Methods for the extraction and purification of nucleic acids are known. For example, nucleic acids can be purified by organic extraction with phenol, phenol/chloroform/isoamyl alcohol, or similar formulations, including TRIzol and TriReagent. Other non-limiting examples of extraction techniques include: (1) organic extraction followed by ethanol precipitation, e.g., using a phenol/chloroform organic reagent (Ausubel et al., 1993), with or without the use of an automated nucleic acid extractor, e.g., the Model 341 DNA Extractor available from Applied Biosystems (Foster City, Calif.); (2) stationary phase adsorption methods (U.S. Pat. No. 5,234,809; Walsh et al., 1991); and (3) salt-induced nucleic acid precipitation methods (Miller et al., (1988), such precipitation methods being typically referred to as “salting-out” methods. Another example of nucleic acid isolation and/or purification includes the use of magnetic particles to which nucleic acids can specifically or non-specifically bind, followed by isolation of the beads using a magnet, and washing and eluting the nucleic acids from the beads (see. e.g., U.S. Pat. No. 5,705,628). In some embodiments, the above isolation methods may be preceded by an enzyme digestion step to help eliminate unwanted protein from the sample, e.g., digestion with proteinase K. or other like proteases (see. e.g., U.S. Pat. No. 7,001,724). If desired, RNase inhibitors may be added to the lysis buffer. For certain cell or sample types, it may be desirable to add a protein denaturation/digestion step to the protocol. Purification methods may be directed to isolate DNA, RNA, or both. When both DNA and RNA are isolated together during or subsequent to an extraction procedure, further steps may be employed to purify one or both separately from the other. Sub-fractions of extracted nucleic acids can also be generated, for example, purification by size, sequence, or other physical or chemical characteristic. In addition to an initial nucleic isolation step, purification of nucleic acids can be performed after any step in the methods of the disclosure, such as to remove excess or unwanted reagents, reactants, or products.

Nucleic acid template molecules can be obtained as described in U.S. Patent Application Publication Number US2002/0190663 A1, published Oct. 9, 2003. Generally, nucleic acid can be extracted from a biological sample by a variety of techniques such as those described by Maniatis, et al., Molecular Cloning: A Laboratory Manual. Cold Spring Harbor, N.Y., pp. 280-281 (1982). In some cases, the nucleic acids can be first extracted from the biological samples and then cross-linked in vitro. In some cases, native association proteins (e.g., histones) can be further removed from the nucleic acids.

In other embodiments, the disclosure can be easily applied to any high molecular weight double stranded DNA including, for example, DNA isolated from tissues, cell culture, bodily fluids, animal tissue, plant, bacteria, fungi, viruses, etc.

In some embodiments, each of the plurality of independent samples can independently comprise at least about 1 ng, 2 ng 0.5 ng, 10 ng, 20 ng, 30 ng, 40 ng, 50 ng, 75 ng, 100 ng, 150 ng, 200 ng, 250 ng, 300 ng, 400 ng, 500 ng, 1 μg, 1.5 μg, 2 μg, 5 μg, 10 μg, 20 μg, 50 μg, 100 μg, 200 μg, 500 μg, or 1000 μg, or more of nucleic acid material. In some embodiments, each of the plurality of independent samples can independently comprise less than about 1 ng, 2 ng, 5 ng, 10 ng, 20 ng, 30 ng, 40 ng, 50 ng, 75 ng, 100 ng, 150 ng, 200 ng, 250 ng, 300 ng, 400 ng, 500 ng, 1 μg, 1.5 μg, 2 μg, 5 μg, 10 μg, 20 μg, 50 μg, 100 μg, 200 μg, 500 μg, or 1000 μg, or more of nucleic acid.

In some embodiments, end repair is performed to generate blunt end 5′ phosphorylated nucleic acid ends using commercial kits, such as those available from Epicentre Biotechnologies (Madison, WI).

### Adaptors

An adaptor oligonucleotide includes any oligonucleotide having a sequence, at least a portion of which is known, that can be joined to a target polynucleotide. Adaptor oligonucleotides can comprise DNA, RNA, nucleotide analogues, non-canonical nucleotides, labeled nucleotides, modified nucleotides, or combinations thereof. Adaptor oligonucleotides can be single-stranded, double-stranded, or partial duplex. In general, a partial-duplex adaptor comprises one or more single-stranded regions and one or more double-stranded regions. Double-stranded adaptors can comprise two separate oligonucleotides hybridized to one another (also referred to as an “oligonucleotide duplex”), and hybridization may leave one or more blunt ends, one or more 3′ overhangs, one or more 5′ overhangs, one or more bulges resulting from mismatched and/or unpaired nucleotides, or any combination of these. In some embodiments, a single-stranded adaptor comprises two or more sequences that are able to hybridize with one another. When two such hybridizable sequences are contained in a single-stranded adaptor, hybridization yields a hairpin structure (hairpin adaptor). When two hybridized regions of an adaptor are separated from one another by a non-hybridized region, a “bubble” structure results. Adaptors comprising a bubble structure can consist of a single adaptor oligonucleotide comprising internal hybridizations, or may comprise two or more adaptor oligonucleotides hybridized to one another. Internal sequence hybridization, such as between two hybridizable sequences in an adaptor, can produce a double-stranded structure in a single-stranded adaptor oligonucleotide. Adaptors of different kinds can be used in combination, such as a hairpin adaptor and a double-stranded adaptor, or adaptors of different sequences. Hybridizable sequences in a hairpin adaptor may or may not include one or both ends of the oligonucleotide. When neither of the ends are included in the hybridizable sequences, both ends are “free” or “overhanging.” When only one end is hybridizable to another sequence in the adaptor, the other end forms an overhang, such as a 3′ overhang or a 5′ overhang. When both the 5′-terminal nucleotide and the 3′-terminal nucleotide are included in the hybridizable sequences, such that the 5′-terminal nucleotide and the 3′-terminal nucleotide are complementary and hybridize with one another, the end is referred to as “blunt.” Different adaptors can be joined to target polynucleotides in sequential reactions or simultaneously. For example, the first and second adaptors can be added to the same reaction. Adaptors can be manipulated prior to combining with target polynucleotides. For example, terminal phosphates can be added or removed.

Adaptors can contain one or more of a variety of sequence elements including, but not limited to, one or more amplification primer annealing sequences or complements thereof, one or more sequencing primer annealing sequences or complements thereof, one or more barcode sequences, one or more common sequences shared among multiple different adaptors or subsets of different adaptors, one or more restriction enzyme recognition sites, one or more overhangs complementary to one or more target polynucleotide overhangs, one or more probe binding sites (e.g., for attachment to a sequencing platform, such as a flow cell for massive parallel sequencing, such as developed by Illumina, Inc.), one or more random or near-random sequences (e.g., one or more nucleotides selected at random from a set of two or more different nucleotides at one or more positions, with each of the different nucleotides selected at one or more positions represented in a pool of adaptors comprising the random sequence), and combinations thereof. Two or more sequence elements can be non-adjacent to one another (e.g., separated by one or more nucleotides), adjacent to one another, partially overlapping, or completely overlapping. For example, an amplification primer annealing sequence can also serve as a sequencing primer annealing sequence. Sequence elements can be located at or near the 3′ end, at or near the 5′ end, or in the interior of the adaptor oligonucleotide. When an adaptor oligonucleotide is capable of forming secondary structure, such as a hairpin, sequence elements can be located partially or completely outside the secondary structure, partially or completely inside the secondary structure, or in between sequences participating in the secondary structure. For example, when an adaptor oligonucleotide comprises a hairpin structure, sequence elements can be located partially or completely inside or outside the hybridizable sequences (the “stem”), including in the sequence between the hybridizable sequences (the “loop”). In some embodiments, the first adaptor oligonucleotides in a plurality of first adaptor oligonucleotides having different barcode sequences comprise a sequence element common among all first adaptor oligonucleotides in the plurality. In some embodiments, all second adaptor oligonucleotides comprise a sequence element common among all second adaptor oligonucleotides that is different from the common sequence element shared by the first adaptor oligonucleotides. A difference in sequence elements can be any such that at least a portion of different adaptors do not completely align, for example, due to changes in sequence length, deletion, or insertion of one or more nucleotides, or a change in the nucleotide composition at one or more nucleotide positions (such as a base change or base modification). In some embodiments, an adaptor oligonucleotide comprises a 5′ overhang, a 3′ overhang, or both that is complementary to one or more target polynucleotides. Complementary overhangs can be one or more nucleotides in length including, but not limited to, 1, 2, 3, 4, 5, 6, 7, 8, 9, 10, 11, 12, 13, 14, 15, or more nucleotides in length. For example, the complementary overhangs can be about 1, 2, 3, 4, 5 or 6 nucleotides in length. Complementary overhangs may comprise a fixed sequence. Complementary overhangs may comprise a random sequence of one or more nucleotides, such that one or more nucleotides are selected at random from a set of two or more different nucleotides at one or more positions, with each of the different nucleotides selected at one or more positions represented in a pool of adaptors with complementary overhangs comprising the random sequence. In some embodiments, an adaptor overhang consists of an adenine or a thymine.

Adaptor oligonucleotides can have any suitable length, at least sufficient to accommodate the one or more sequence elements of which they are comprised. In some embodiments, adaptors are about, less than about, or more than about, 10, 15, 20, 25, 30, 35, 40, 45, 50, 55, 60, 65, 70, 75, 80, 90, 100, 200, or more nucleotides in length. In some examples, the adaptors can be about 10 to about 50 nucleotides in length. In further examples, the adaptors can be about 20 to about 40 nucleotides in length.

As used herein, the terms “barcode” or “unique molecular identifier (UMI)” used interchangeably herein, refers to a known nucleic acid sequence that allows some feature of a polynucleotide with which the barcode is associated to be identified. In some embodiments, the feature of the polynucleotide to be identified is the sample from which the polynucleotide is derived. In some embodiments, barcodes can be at least 3, 4, 5, 6, 7, 8, 9, 10, 11, 12, 13, 14, 15, or more nucleotides in length. For example, barcodes can be at least 10, 11, 12, 13, 14, or 15 nucleotides in length. In some embodiments, barcodes can be shorter than 10, 9, 8, 7, 6, 5, or 4 nucleotides in length. For example, barcodes can be shorter than 10 nucleotides in length. In some embodiments, barcodes associated with some polynucleotides are of different length than barcodes associated with other polynucleotides. In general, barcodes are of sufficient length and comprise sequences that are sufficiently different to allow the identification of samples based on barcodes with which they are associated. In some embodiments, a barcode, and the sample source with which it is associated, can be identified accurately after the mutation, insertion, or deletion of one or more nucleotides in the barcode sequence, such as the mutation, insertion, or deletion of 1, 2, 3, 4, 5, 6, 7, 8, 9, 10, or more nucleotides. In some examples, 1, 2 or 3 nucleotides can be mutated, inserted and/or deleted. In some embodiments, each barcode in a plurality of barcodes differ from every other barcode in the plurality at least two nucleotide positions, such as at least 2, 3, 4, 5, 6, 7, 8, 9, 10, or more positions. In some examples, each barcode can differ from every other barcode by in at least 2, 3, 4 or 5 positions. In some embodiments, both a first site and a second site comprise at least one of a plurality of barcode sequences. In some embodiments, barcodes for second sites are selected independently from barcodes for first adaptor oligonucleotides. In some embodiments, first sites and second sites having barcodes are paired, such that sequences of the pair comprise the same or different one or more barcodes. In some embodiments, the methods of the disclosure further comprise identifying the sample from which a target polynucleotide is derived based on a barcode sequence to which the target polynucleotide is joined. In general, a barcode may comprise a nucleic acid sequence that when joined to a target polynucleotide serves as an identifier of the sample from which the target polynucleotide was derived.

Adaptor oligonucleotides may be coupled, linked, or tethered to an immunoglobulin or an immunoglobulin binding protein or fragment thereof. For example, after in situ genomic digestion of a crosslinked sample with a DNase, such as MNase, one or more antibodies may be added to the sample to bind the digested chromatin, such as at methylated sites or transcription factor binding sites. Next, a biotinylated adaptor oligonucleotide coupled, linked, or tethered to an immunoglobulin binding protein or fragment thereof, such as a Protein A, a Protein G, a Protein A/G. or a Protein L, may be added to the sample to target the adaptors to one or more specific sites in the chromatin. Adaptor oligonucleotides provided herein can also comprise DBCO moieties to enable click chemistry modifications to the adaptor. The sample may then be treated with a ligase to effect proximity ligation. Moreover, streptavidin may be used to isolate DNA that has been ligated to the adaptors. Crosslinks may then be reversed before amplifying the sample using PCR and sequencing. Alternatively, adaptor linked oligonucleotides may comprise modified nucleotides capable of linking to a purification reagent using click chemistry.

### Nucleic Acids

In eukaryotes, genomic DNA is packed into chromatin to consist as chromosomes within the nucleus. The basic structural unit of chromatin is the nucleosome, which consists of 146 base pairs (bp) of DNA wrapped around a histone octamer. The histone octamer consists of two copies each of the core histone H2A-H2B dimers and H3-H4 dimers. Nucleosomes are regularly spaced along the DNA in what is commonly referred to as “beads on a string.”

The assembly of core histones and DNA into nucleosomes is mediated by chaperone proteins and associated assembly factors. Nearly all of these factors are core histone-binding proteins. Some of the histone chaperones, such as nucleosome assembly protein-1 (NAP-1), exhibit a preference for binding to histones H3 and H4. It has also been observed that newly synthesized histones are acetylated and then subsequently deacetylated after assembly into chromatin. The factors that mediate histone acetylation or deacetylation therefore play an important role in the chromatin assembly process.

In particular embodiments, the methods of the disclosure can be easily applied to any type of nucleic acid, such as double stranded DNA including, but not limited to, for example, chromatin isolated from cells or within a cell or nucleus; cDNA; free DNA isolated from plasma, serum, and/or urine; apoptotic DNA from cells and/or tissues; and/or DNA fragmented enzymatically in vitro (for example, by MNase or DNase I). In some cases, RNA is used.

For some applications, nucleic acid obtained from biological samples can be fragmented to produce suitable fragments for analysis. In some cases, polynucleotides are bound to a dendrimer prior to fragmentation. Alternatively, polynucleotides are fragmented prior to binding with a dendrimer. Template nucleic acids may be fragmented to desired length, using a variety of enzymatic methods. DNA may be randomly sheared brief exposure to a DNase. RNA may be fragmented by brief exposure to an RNase, heat plus magnesium, or by shearing. The RNA may be converted to cDNA. If fragmentation is employed, the RNA may be converted to cDNA before or after fragmentation. Nucleic acid molecules may be single-stranded, double-stranded, or double-stranded with single-stranded regions (for example, stem- and loop-structures).

When fragmented DNA is used, cross-linked DNA molecules may be subjected to a size selection step. Size selection of the nucleic acids may be performed to cross-linked DNA molecules below or above a certain size. Size selection may further be affected by the frequency of cross-links and/or by the fragmentation method. In some embodiments, a composition may be prepared comprising cross-linking a DNA molecule in the range of about 145 bp to about 600 bp, about 100 bp to about 2500 bp, about 600 to about 2500 bp, about 350 bp to about 1000 bp, or any range bounded by any of these values (e.g., about 100 bp to about 2500 bp).

In some embodiments, sample polynucleotides are fragmented into a population of fragmented DNA molecules of one or more specific size range(s). In some embodiments, fragments can be generated from at least about 1, about 2, about 5, about 10, about 20, about 50, about 100, about 200, about 500, about 1000, about 2000, about 5000, about 10,000, about 20,000, about 50,000, about 100,000, about 200,000, about 500,000, about 1,000,000, about 2,000,000, about 5,000,000, about 10,000,000, or more genome-equivalents of starting DNA. Fragmentation may be accomplished by DNase treatment. In some embodiments, the fragments have an average length from about 10 to about 10,000, about 20,000, about 30,000, about 40,000, about 50,000, about 60,000, about 70,000, about 80,000, about 90,000, about 100,000, about 150,000, about 200,000, about 300,000, about 400,000, about 500,000, about 600,000, about 700,000, about 800,000, about 900,000, about 1,000,000, about 2,000,000, about 5,000,000, about 10,000,000, or more nucleotides. In some embodiments, the fragments have an average length from about 145 bp to about 600 bp, about 100 bp to about 2500 bp, about 600 to about 2500 bp, about 350 bp to about 1000 bp, or any range bounded by any of these values (e.g., about 100 bp to about 2500 bp). In some embodiments, the fragments have an average length less than about 2500 bp, less than about 1200 bp, less than about 1000 bp, less than about 800 bp, less than about 600 bp, less than about 350 bp, or less than about 200 bp. In other embodiments, the fragments have an average length more than about 110 bp, more than about 350 bp, more than about 600 bp, more than about 800 bp, more than about 1000 bp, more than about 1200 bp, or more than about 2000 bp. Non-limiting examples of DNases include DNase I, DNase II, micrococcal nuclease, variants thereof, and combinations thereof. For example, digestion with DNase I can induce random double-stranded breaks in DNA in the absence of Mg++ and in the presence of Mn++. Fragmentation can produce fragments having 5′ overhangs, 3′ overhangs, blunt ends, or a combination thereof. In some embodiments, the method includes the step of size selecting the fragments via standard methods such as column purification or isolation from an agarose gel.

### Ligation

In some embodiments, the 5′ and/or 3′ end nucleotide sequences of fragmented DNA are not modified prior to ligation. For example, cleavage by an enzyme that leaves a predictable blunt end can be followed by ligation of blunt-ended DNA fragments to nucleic acids, such as adaptors, oligonucleotides, or polynucleotides, comprising a blunt end. In some embodiments, the fragmented DNA molecules are blunt-end polished (or “end repaired”) to produce DNA fragments having blunt ends, prior to being joined to adaptors. The blunt-end polishing step may be accomplished by incubation with a suitable enzyme, such as a DNA polymerase that has both 3′ to 5′ exonuclease activity and 5′ to 3′ polymerase activity, for example, T4 polymerase. In some embodiments, end repair can be followed by an addition of 1, 2, 3, 4, 5, 6, 7, 8, 9, 10, 11, 12, 13, 14, 15, 16, 17, 18, 19, 20 or more nucleotides, such as one or more adenine, one or more thymine, one or more guanine, or one or more cytosine, to produce an overhang. For example, the end pair can be followed by an addition of 1, 2, 3, 4, 5, or 6 nucleotides. DNA fragments having an overhang can be joined to one or more nucleic acids, such as oligonucleotides, adaptor oligonucleotides, or polynucleotides, having a complementary overhang, such as in a ligation reaction. For example, a single adenine can be added to the 3′ ends of end repaired DNA fragments using a template independent polymerase, followed by ligation to one or more adaptors each having a thymine at a 3′ end. In some embodiments, nucleic acids, such as oligonucleotides or polynucleotides can be joined to blunt end double-stranded DNA molecules which have been modified by extension of the 3′ end with one or more nucleotides followed by 5′ phosphorylation. In some cases, extension of the 3′ end may be performed with a polymerase such as, Klenow polymerase or any of the suitable polymerases provided herein, or by use of a terminal deoxynucleotide transferase, in the presence of one or more dNTPs in a suitable buffer that can contain magnesium. In some embodiments, target polynucleotides having blunt ends are joined to one or more adaptors comprising a blunt end. Phosphorylation of 5′ ends of DNA fragment molecules may be performed, for example, with T4 polynucleotide kinase in a suitable buffer containing ATP and magnesium. The fragmented DNA molecules may optionally be treated to dephosphorylate 5′ ends or 3′ ends, for example, by using enzymes such as phosphatases.

The terms “connecting,” “joining,” and “ligation” as used herein, with respect to two polynucleotides, such as an adaptor oligonucleotide and a target poly nucleotide, refers to the covalent attachment of two separate DNA segments to produce a single larger polynucleotide with a contiguous backbone. Methods for joining two DNA segments include, without limitation, enzymatic and non-enzymatic (e.g., chemical) methods. Examples of ligation reactions that are non-enzymatic include the non-enzymatic ligation techniques described in U.S. Pat. Nos. 5,780,613 and 5,476,930, which are herein incorporated by reference. In some embodiments, an adaptor oligonucleotide is joined to a target polynucleotide by a ligase, for example, a DNA ligase or RNA ligase. Multiple ligases, each having characterized reaction conditions include, without limitation. NAD+-dependent ligases including tRNA ligase, Taq DNA ligase, Thermus filiformis DNA ligase. Escherichia coli DNA ligase, Tth DNA ligase. Thermus scotoductus DNA ligase (I and II), thermostable ligase. Ampligase thermostable DNA ligase. VanC-type ligase, 9° N DNA Ligase, Tsp DNA ligase, and novel ligases discovered by bioprospecting; ATP-dependent ligases including T4 RNA ligase, T4 DNA ligase. T3 DNA ligase, T7 DNA ligase, Pfu DNA ligase, DNA ligase 1, DNA ligase III, DNA ligase IV, and novel ligases discovered by bioprospecting; and wild-type, mutant isoforms, and genetically engineered variants thereof.

Ligation can be between DNA segments having hybridizable sequences, such as complementary overhangs. Ligation can also be between two blunt ends. Generally, a 5′ phosphate is utilized in a ligation reaction. The 5′ phosphate can be provided by the target polynucleotide, the adaptor oligonucleotide, or both, 5′ phosphates can be added to or removed from DNA segments to be joined, as needed. Methods for the addition or removal of 5′ phosphates include, without limitation, enzymatic and chemical processes. Enzymes useful in the addition and/or removal of 5′ phosphates include kinases, phosphatases, and polymerases. In some embodiments, both of the two ends joined in a ligation reaction (e.g., an adaptor end and a target polynucleotide end) provide a 5′ phosphate, such that two covalent linkages are made in joining the two ends. In some embodiments, only one of the two ends joined in a ligation reaction (e.g., only one of an adaptor end and a target polynucleotide end) provides a 5′ phosphate, such that only one covalent linkage is made in joining the two ends.

In some embodiments, only one strand at one or both ends of a target polynucleotide is joined to an adaptor oligonucleotide. In some embodiments, both strands at one or both ends of a target polynucleotide are joined to an adaptor oligonucleotide. In some embodiments, 3′ phosphates are removed prior to ligation. In some embodiments, an adaptor oligonucleotide is added to both ends of a target polynucleotide, wherein one or both strands at each end are joined to one or more adaptor oligonucleotides. When both strands at both ends are joined to an adaptor oligonucleotide, joining can be followed by a cleavage reaction that leaves a 5′ overhang that can serve as a template for the extension of the corresponding 3′ end, which 3′ end may or may not include one or more nucleotides derived from the adaptor oligonucleotide. In some embodiments, a target polynucleotide is joined to a first adaptor oligonucleotide on one end and a second adaptor oligonucleotide on the other end. In some embodiments, two ends of a target polynucleotide are joined to the opposite ends of a single adaptor oligonucleotide. In some embodiments, the target polynucleotide and the adaptor oligonucleotide to which it is joined comprise blunt ends. In some embodiments, separate ligation reactions can be carried out for each sample, using a different first adaptor oligonucleotide comprising at least one barcode sequence for each sample, such that no barcode sequence is joined to the target polynucleotides of more than one sample. A DNA segment or a target polynucleotide that has an adaptor oligonucleotide joined to it is considered “tagged” by the joined adaptor.

In some cases, the ligation reaction can be performed at a DNA segment or target polynucleotide concentration of about 0.1 ng/μL, about 0.2 ng/μL, about 0.3 ng/μL, about 0.4 ng/μL, about 0.5 ng/μL, about 0.6 ng/μL, about 0.7 ng/μL, about 0.8 ng/μL, about 0.9 ng/μL, about 1.0 ng/μL, about 1.2 ng/μL, about 1.4 ng/μL, about 1.6 ng/μL, about 1.8 ng/μL, about 2.0 ng/μL, about 2.5 ng/μL, about 3.0 ng/μL, about 3.5 ng/μL, about 4.0 ng/μL, about 4.5 ng/μL, about 5.0 ng/μL, about 6.0 ng/μL, about 7.0 ng/μL, about 8.0 ng/μL, about 9.0 ng/μL, about 10 ng/μL, about 15 ng/μL, about 20 ng/μL, about 30 ng/μL, about 40 ng/μL, about 50 ng/μL, about 60 ng/μL, about 70 ng/μL, about 80 ng/μL, about 90 ng/μL, about 100 ng/μL, about 150 ng/μL, about 200 ng/μL, about 300 ng/μL, about 400 ng/μL, about 500 ng/μL, about 600 ng/μL, about 800 ng/μL, or about 1000 ng/μL. For example, the ligation can be performed at a DNA segment or target polynucleotide concentration of about 100 ng/μL, about 150 ng/μL, about 200 ng/μL, about 300 ng/μL, about 400 ng/μL, or about 500 ng/μL.

In some cases, the ligation reaction can be performed at a DNA segment or target polynucleotide concentration of about 0.1 to 1000 ng/μL, about 1 to 1000 ng/μL, about 1 to 800 ng/μL, about 10 to 800 ng/μL, about 10 to 600 ng/μL, about 100 to 600 ng/μL, or about 100 to 500 ng/μL.

In some cases, the ligation reaction can be performed for more than about 5 minutes, about 10 minutes, about 20 minutes, about 30 minutes, about 40 minutes, about 50 minutes, about 60 minutes, about 90 minutes, about 2 hours, about 3 hours, about 4 hours, about 5 hours, about 6 hours, about 8 hours, about 10 hours, about 12 hours, about 18 hours, about 24 hours, about 36 hours, about 48 hours, or about 96 hours. In other cases, the ligation reaction can be performed for less than about 5 minutes, about 10 minutes, about 20 minutes, about 30 minutes, about 40 minutes, about 50 minutes, about 60 minutes, about 90 minutes, about 2 hours, about 3 hours, about 4 hours, about 5 hours, about 6 hours, about 8 hours, about 10 hours, about 12 hours, about 18 hours, about 24 hours, about 36 hours, about 48 hours, or about 96 hours. For example, the ligation reaction can be performed for about 30 minutes to about 90 minutes. In some embodiments, joining of an adaptor to a target polynucleotide produces a joined product polynucleotide having a 3′ overhang comprising a nucleotide sequence derived from the adaptor.

In some embodiments, after joining at least one adaptor oligonucleotide to a target polynucleotide, the 3′ end of one or more target polynucleotides is extended using the one or more joined adaptor oligonucleotides as template. For example, an adaptor comprising two hybridized oligonucleotides that is joined to only the 5′ end of a target polynucleotide allows for the extension of the unjoined 3′ end of the target using the joined strand of the adaptor as template, concurrently with or following displacement of the unjoined strand. Both strands of an adaptor comprising two hybridized oligonucleotides may be joined to a target polynucleotide such that the joined product has a 5′ overhang, and the complementary 3′ end can be extended using the 5′ overhang as template. As a further example, a hairpin adaptor oligonucleotide can be joined to the 5′ end of a target polynucleotide. In some embodiments, the 3′ end of the target polynucleotide that is extended comprises one or more nucleotides from an adaptor oligonucleotide. For target polynucleotides to which adaptors are joined on both ends, extension can be carried out for both 3′ ends of a double-stranded target polynucleotide having 5′ overhangs. This 3′ end extension, or “fill-in” reaction, generates a complementary sequence, or “complement,” to the adaptor oligonucleotide template that is hybridized to the template, thus filling in the 5′ overhang to produce a double-stranded sequence region. Where both ends of a double-stranded target polynucleotide have 5′ overhangs that are filled in by extension of the complementary strands' 3′ ends, the product is completely double-stranded. Extension can be carried out by any suitable polymerase, such as a DNA polymerase, many of which are commercially available. DNA polymerases can comprise DNA-dependent DNA polymerase activity, RNA-dependent DNA poly merase activity, or DNA-dependent and RNA-dependent DNA polymerase activity. DNA polymerases can be thermostable or non-thermostable. Examples of DNA polymerases include, but are not limited to, Taq polymerase, Tth polymerase, Tli polymerase, Pfu polymerase, Pfutubo polymerase, Pyrobest polymerase, Pwo polymerase, KOD polymerase. Bst polymerase, Sac polymerase. Sso polymerase, Poc polymerase, Pab polymerase. Mth polymerase. Pho polymerase, ES4 polymerase. VENT polymerase, DEEPVENT poly merase, EX-Taq poly merase, LA-Taq polymerase, Expand polymerases. Platinum Taq poly merases, Hi-Fi polymerase, Tbr polymerase, Tfl polymerase, Tru polymerase, Tac polymerase, Tne polymerase, Tma polymerase. Tih polymerase, Tfi polymerase, Klenow fragment, and variants, modified products and derivatives thereof 3′ end extension can be performed before or after pooling of target polynucleotides from independent samples.

### Target Enrichment

In certain embodiments, the disclosure provides methods for the enrichment of a target nucleic acids and analysis of the target nucleic acids. In some cases, the method for enrichment is in a solution-based format. In some cases, the target nucleic acid can be labeled with a labeling agent. In other cases, the target nucleic acid can be crosslinked to one or more association molecules that are labeled with a labeling agent. Examples of labeling agents include, but are not limited to, biotin, polyhistidine tags, and chemical tags (e.g., alkyne and azide derivatives used in Click Chemistry methods). Further, the labeled target nucleic acid can be captured and thereby enriched by using a capturing agent. The capturing agent can be streptavidin and/or avidin, an antibody, a chemical moiety (e.g., alkyne, azide), and any biological, chemical, physical, or enzymatic agents used for affinity purification.

In some cases, immobilized or non-immobilized nucleic acid probes can be used to capture the target nucleic acids. For example, the target nucleic acids can be enriched from a sample by hybridization to the probes on a solid support or in solution. In some examples, the sample can be a genomic sample. In some examples, the probes can be an amplicon. The amplicon can comprise a predetermined sequence. Further, the hybridized target nucleic acids can be washed and/or eluted off of the probes. The target nucleic acid can be a DNA, RNA, cDNA, or mRNA molecule.

In some cases, the enrichment method can comprise contacting the sample comprising the target nucleic acid to the probes and binding the target nucleic acid to a solid support. In some cases, the sample can be fragmented using enzymatic methods to yield the target nucleic acids. In some cases, the probes can be specifically hybridized to the target nucleic acids. In some cases, the target nucleic acids can have an average size of about 145 bp to about 600 bp, about 100 bp to about 2500 bp, about 600 to about 2500 bp, or about 350 bp to about 1000 bp. The target nucleic acids can be further separated from the unbound nucleic acids in the sample. The solid support can be washed and/or eluted to provide the enriched target nucleic acids. In some examples, the enrichment steps can be repeated for about 1, 2, 3, 4, 5, 6, 7, 8, 9, or 10 times. For example, the enrichment steps can be repeated for about 1, 2, or 3 times.

In some cases, the enrichment method can comprise providing probe derived amplicons wherein said probes for amplification are attached to a solid support. The solid support can comprise support-immobilized nucleic acid probes to capture specific target nucleic acid from a sample. The probe derived amplicons can hybridize to the target nucleic acids. Following hybridization to the probe amplicons, the target nucleic acids in the sample can be enriched by capturing (e.g., via capturing agents as biotin, antibodies, etc.) and washing and/or eluting the hybridized target nucleic acids from the captured probes. The target nucleic acid sequence(s) may be further amplified using, for example, PCR methods to produce an amplified pool of enriched PCR products.

In some cases, the solid support can be a microarray, a slide, a chip, a microwell, a column, a tube, a particle, or a bead. In some examples, the solid support can be coated with streptavidin and/or avidin. In other examples, the solid support can be coated with an antibody. Further, the solid support can comprise a glass, metal, ceramic or polymeric material. In some embodiments, the solid support can be a nucleic acid microarray (e.g., a DNA microarray). In other embodiments, the solid support can be a paramagnetic bead.

In particular embodiments, the disclosure provides methods for amplifying the enriched DNA. In some cases, the enriched DNA is a read-pair. The read-pair can be obtained by the methods of the present disclosure.

In some embodiments, the one or more amplification and/or replication steps are used for the preparation of a library to be sequenced. Any suitable amplification method may be used. Examples of amplification techniques that can be used include, but are not limited to, quantitative PCR, quantitative fluorescent PCR (QF-PCR), multiplex fluorescent PCR (MF-PCR), real time PCR (RTPCR), single cell PCR, restriction fragment length polymorphism PCR (PCR-RFLP). PCK-RFLPIRT-PCR-IRFLP, hot start PCR, nested PCR, in situ polonony PCR, in situ rolling circle amplification (RCA), bridge PCR, ligation mediated PCR. Qb replicase amplification, inverse PCR, picotiter PCR and emulsion PCR. Other suitable amplification methods include the ligase chain reaction (LCR), transcription amplification, self-sustained sequence replication, selective amplification of target polynucleotide sequences, consensus sequence primed poly merase chain reaction (CP-PCR), arbitrarily primed polymerase chain reaction (AP-PCR), degenerate oligonucleotide-primed PCR (DOP-PCR) and nucleic acid-based sequence amplification (NABSA). Other amplification methods that can be used herein include those described in U.S. Pat. Nos. 5,242,794; 5,494,810; 4,988,617; and 6,582,938.

In particular embodiments, PCR is used to amplify DNA molecules after they are dispensed into individual partitions. In some cases, one or more specific priming sequences within amplification adaptors are utilized for PCR amplification. The amplification adaptors may be ligated to fragmented DNA molecules before or after dispensing into individual partitions. Polynucleotides comprising amplification adaptors with suitable priming sequences on both ends can be PCR amplified exponentially. Polynucleotides with only one suitable priming sequence due to, for example, imperfect ligation efficiency of amplification adaptors comprising priming sequences, may only undergo linear amplification. Further, polynucleotides can be eliminated from amplification, for example, PCR amplification, all together, if no adaptors comprising suitable priming sequences are ligated. In some embodiments, the number of PCR cycles vary between 10-30, but can be as low as 9, 8, 7, 6, 5, 4, 3, 2 or less or as high as 40, 45, 50, 55, 60 or more. As a result, exponentially amplifiable fragments carrying amplification adaptors with a suitable priming sequence can be present in much higher (1000 fold or more) concentration compared to linearly amplifiable or un-amplifiable fragments, after a PCR amplification. Benefits of PCR, as compared to whole genome amplification techniques (such as amplification with randomized primers or Multiple Displacement Amplification using phi29 polymerase) include, but are not limited to, a more uniform relative sequence coverage—as each fragment can be copied at most once per cycle and as the amplification is controlled by thermocycling program, a substantially lower rate of forming chimeric molecules than, for example, MDA (Lasken et al., 2007, BMC Biotechnology)—as chimeric molecules pose significant challenges for accurate sequence assembly by presenting nonbiological sequences in the assembly graph, which may result in higher rate of misassemblies or highly ambiguous and fragmented assembly, reduced sequence specific biases that may result from binding of randomized primers commonly used in MDA versus using specific priming sites with a specific sequence, a higher reproducibility in the amount of final amplified DNA product, which can be controlled by selection of the number of PCR cycles, and a higher fidelity in replication with the polymerases that are commonly used in PCR as compared to common whole genome amplification techniques.

In some embodiments, the fill-in reaction is followed by or performed as part of amplification of one or more target polynucleotides using a first primer and a second primer, wherein the first primer comprises a sequence that is hybridizable to at least a portion of the complement of one or more of the first adaptor oligonucleotides, and further wherein the second primer comprises a sequence that is hybridizable to at least a portion of the complement of one or more of the second adaptor oligonucleotides. Each of the first and second primers may be of any suitable length, such as about, less than about, or more than about 10, 15, 20, 25, 30, 35, 40, 45, 50, 55, 60, 65, 70, 75, 80, 90, 100, or more nucleotides, any portion or all of which may be complementary to the corresponding target sequence (e.g., about, less than about, or more than about 5, 10, 15, 20, 25, 30, 35, 40, 45, 50, or more nucleotides). For example, about 10 to 50 nucleotides can be complementary to the corresponding target sequence.

“Amplification” refers to any process by which the copy number of a target sequence is increased. In some cases, a replication reaction may produce only a single complementary copy/replica of a polynucleotide. Methods for primer-directed amplification of target polynucleotides include, without limitation, methods based on the polymerase chain reaction (PCR). Conditions favorable to the amplification of target sequences by PCR can be optimized at a variety of steps in the process, and depend on characteristics of elements in the reaction, such as target type, target concentration, sequence length to be amplified, sequence of the target and/or one or more primers, primer length, primer concentration, polymerase used, reaction volume, ratio of one or more elements to one or more other elements, and others, some, or all of which can be altered. In general, PCR involves the steps of denaturation of the target to be amplified (if double stranded), hybridization of one or more primers to the target, and extension of the primers by a DNA polymerase, with the steps repeated (or “cycled”) in order to amplify the target sequence. Steps in this process can be optimized for various outcomes, such as to enhance yield, decrease the formation of spurious products, and/or increase or decrease specificity of primer annealing. Methods of optimization include, without limitation, adjustments to the type or number of elements in the amplification reaction and/or to the conditions of a given step in the process, such as temperature at a particular step, duration of a particular step, and/or number of cycles.

In some embodiments, an amplification reaction can comprise at least about 5, 10, 15, 20, 25, 30, 35, 40, 50, 60, 70, 80, 90, 100, 150, 200 or more cycles. In some examples, an amplification reaction can comprise at least about 20, 25, 30, 35 or 40 cycles. In some embodiments, an amplification reaction comprises no more than about 5, 10, 15, 20, 25, 35, 40, 50, 60, 70, 80, 90, 100, 150, 200 or more cycles. Cycles can contain any number of steps, such as 1, 2, 3, 4, 5, 6, 7, 8, 9, 10 or more steps. Steps can comprise any temperature or gradient of temperatures, suitable for achieving the purpose of the given step including, but not limited to, 3′ end extension (e.g., adaptor fill-in), primer annealing, primer extension, and strand denaturation. Steps can be of any duration including, but not limited to, about, less than about, or more than about 1, 5, 10, 15, 20, 25, 30, 35, 40, 45, 50, 55, 60, 70, 80, 90, 100, 120, 180, 240, 300, 360, 420, 480, 540, 600, 1200, 1800, or more seconds, including indefinitely until manually interrupted. Cycles of any number comprising different steps can be combined in any order. In some embodiments, different cycles comprising different steps are combined such that the total number of cycles in the combination is about, less that about, or more than about 5, 10, 15, 20, 25, 30, 35, 40, 50, 60, 70, 80, 90, 100, 150, 200 or more cycles. In some embodiments, amplification is performed following the fill-in reaction.

In some embodiments, the amplification reaction can be carried out on at least about 1, 2, 3, 4, 5, 6, 7, 8, 9, 10, 12, 14, 16, 18, 20, 25, 30, 40, 50, 100, 200, 300, 400, 500, 600, 800, 1000 ng of the target DNA molecule. In other embodiments, the amplification reaction can be carried out on less than about 1, 2, 3, 4, 5, 6, 7, 8, 9, 10, 12, 14, 16, 18, 20, 25, 30, 40, 50, 100, 200, 300, 400, 500, 600, 800, 1000 ng of the target DNA molecule.

Amplification can be performed before or after pooling of target polynucleotides from independent samples.

Methods of the disclosure involve determining an amount of amplifiable nucleic acid present in a sample. Any known method may be used to quantify amplifiable nucleic acid, and an exemplary method is the polymerase chain reaction (PCR), specifically quantitative polymerase chain reaction (qPCR), qPCR is a technique based on the polymerase chain reaction, and is used to amplify and simultaneously quantify a targeted nucleic acid molecule, qPCR allows for both detection and quantification (as absolute number of copies or relative amount when normalized to DNA input or additional normalizing genes) of a specific sequence in a DNA sample. The procedure follows the general principle of polymerase chain reaction, with the additional feature that the amplified DNA is quantified as it accumulates in the reaction in real time after each amplification cycle. QPCR is described, for example, in Kurnit et al. (U.S. Pat. No. 6,033,854), Wang et al. (U.S. Pat. Nos. 5,567,583 and 5,348,853), Ma et al. (The Journal of American Science, 2(3), 2006). Heid et al. (Genome Research 986-994, 1996). Sambrook and Russell (Quantitative PCR, Cold Spring Harbor Protocols, 2006), and Higuchi (U.S. Pat. Nos. 6,171,785 and 5,994,056). The contents of these are incorporated by reference herein in their entirety.

Other methods of quantification include use of fluorescent dyes that intercalate with double-stranded DNA, and modified DNA oligonucleotide probes that fluoresce when hybridized with a complementary DNA. These methods can be broadly used but are also specifically adapted to real-time PCR as described in further detail as an example. In the first method, a DNA-binding dye binds to all double-stranded (ds)DNA in PCR, resulting in fluorescence of the dye. An increase in DNA product during PCR therefore leads to an increase in fluorescence intensity and is measured at each cycle, thus allowing DNA concentrations to be quantified. The reaction is prepared similarly to a standard PCR reaction, with the addition of fluorescent (ds)DNA dye. The reaction is run in a thermocycler, and after each cycle, the levels of fluorescence are measured with a detector, the dye only fluoresces when bound to the (ds)DNA (i.e., the PCR product). With reference to a standard dilution, the (ds)DNA concentration in the PCR can be determined. Like other real-time PCR methods, the values obtained do not have absolute units associated with it. A comparison of a measured DNA/RNA sample to a standard dilution gives a fraction or ratio of the sample relative to the standard, allowing relative comparisons between different tissues or experimental conditions. To ensure accuracy in the quantification and/or expression of a target gene can be normalized with respect to a stably expressed gene. Copy numbers of unknown genes can similarly be normalized relative to genes of known copy number.

The second method uses a sequence-specific RNA or DNA-based probe to quantify only the DNA containing a probe sequence; therefore, use of the reporter probe significantly increases specificity, and allows quantification even in the presence of some non-specific DNA amplification. This allows for multiplexing, i.e., assaying for several genes in the same reaction by using specific probes with differently colored labels, provided that all genes are amplified with similar efficiency.

This method is commonly carried out with a DNA-based probe with a fluorescent reporter (e.g., 6-carboxyfluorescein) at one end and a quencher (e.g., 6-carboxy-tetramethylrhodamine) of fluorescence at the opposite end of the probe. The close proximity of the reporter to the quencher prevents detection of its fluorescence. Breakdown of the probe by the 5′ to 3′ exonuclease activity of a polymerase (e.g., Taq polymerase) breaks the reporter-quencher proximity and thus allows unquenched emission of fluorescence, which can be detected. An increase in the product targeted by the reporter probe at each PCR cycle results in a proportional increase in fluorescence due to breakdown of the probe and release of the reporter. The reaction is prepared similarly to a standard PCR reaction, and the reporter probe is added. As the reaction commences, during the annealing stage of the PCR both probe and primers anneal to the DNA target. Polymerization of a new DNA strand is initiated from the primers, and once the polymerase reaches the probe, its 5′-3′-exonuclease degrades the probe, physically separating the fluorescent reporter from the quencher, resulting in an increase in fluorescence. Fluorescence is detected and measured in a real-time PCR thermocycler, and geometric increase of fluorescence corresponding to exponential increase of the product is used to determine the threshold cycle in each reaction.

Relative concentrations of DNA present during the exponential phase of the reaction are determined by plotting fluorescence against cycle number on a logarithmic scale (so an exponentially increasing quantity will give a straight line). A threshold for detection of fluorescence above background is determined. The cycle at which the fluorescence from a sample crosses the threshold is called the cycle threshold. Ct. Since the quantity of DNA doubles every cycle during the exponential phase, relative amounts of DNA can be calculated. e.g., a sample with a Ct of 3 cycles earlier than another has 23=8 times more template. Amounts of nucleic acid (e.g., RNA or DNA) are then determined by comparing the results to a standard curve produced by a real-time PCR of serial dilutions (e.g., undiluted, 1:4, 1:16, 1:64) of a known amount of nucleic acid.

In certain embodiments, the qPCR reaction involves a dual fluorophore approach that takes advantage of fluorescence resonance energy transfer (FRET). e.g., LIGHTCYCLER hybridization probes, where two oligonucleotide probes anneal to the amplicon (see, e.g., U.S. Pat. No. 6,174,670). The oligonucleotides are designed to hybridize in a head-to-tail orientation with the fluorophores separated at a distance that is compatible with efficient energy transfer. Other examples of labeled oligonucleotides that are structured to emit a signal when bound to a nucleic acid or incorporated into an extension product include: SCORPIONS probes (e.g., Whitcombe et al., Nature Biotechnology 17:804-807, 1999, and U.S. Pat. No. 6,326,145). Sunrise (or AMPLIFLOUR) primers (e.g., Nazarenko et al., Nuc. Acids Res. 25:2516-2521, 1997, and U.S. Pat. No. 6,117,635), and LUX primers and MOLECULAR BEACONS probes (e.g., Tyagi et al., Nature Biotechnology 14:303-308, 1996 and U.S. Pat. No. 5,989,823).

In other embodiments, a qPCR reaction uses fluorescent Taqman methodology and an instrument capable of measuring fluorescence in real time (e.g., ABI Prism 7700 Sequence Detector). The Taqman reaction uses a hybridization probe labeled with two different fluorescent dyes. One dye is a reporter dye (6-carboxyfluorescein), the other is a quenching dye (6-carboxy-tetramethylrhodamine). When the probe is intact, fluorescent energy transfer occurs and the reporter dye fluorescent emission is absorbed by the quenching dye. During the extension phase of the PCR cycle, the fluorescent hybridization probe is cleaved by the 5′-3′ nucleolytic activity of the DNA polymerase. On cleavage of the probe, the reporter dye emission is no longer transferred efficiently to the quenching dye, resulting in an increase of the reporter dye fluorescent emission spectra. Any nucleic acid quantification method, including real-time methods or single-point detection methods may be used to quantify the amount of nucleic acid in the sample. The detection can be performed by several different methodologies (e.g., staining, hybridization with a labeled probe; incorporation of biotinylated primers followed by avidin-enzyme conjugate detection; incorporation of 32P-labeled deoxynucleotide triphosphates, such as dCTP or dATP, into the amplified segment), as well as any other suitable detection method for nucleic acid quantification. The quantification may or may not include an amplification step.

In some embodiments, the disclosure provides labels for identifying or quantifying the linked DNA segments. In some cases, the linked DNA segments can be labeled in order to assist in downstream applications, such as array hybridization. For example, the linked DNA segments can be labeled using random priming or nick translation.

A wide variety of labels (e.g., reporters) may be used to label the nucleotide sequences described herein including, but not limited to, during the amplification step. Suitable labels include radionuclides, enzymes, fluorescent, chemiluminescent, or chromogenic agents as well as ligands, cofactors, inhibitors, magnetic particles, and the like. Examples of such labels are included in U.S. Pat. Nos. 3,817,837; 3,850,752; 3,939,350; 3,996,345; 4,277,437; 4,275,149 and 4,366,241, which are incorporated by reference in its entirety.

Additional labels include, but are not limited to, β-galactosidase, invertase, green fluorescent protein, luciferase, chloramphenicol, acetyltransferase, β-glucuronidase, exo-glucanase and glucoamylase. Fluorescent labels may also be used, as well as fluorescent reagents specifically synthesized with particular chemical properties. A wide variety of ways to measure fluorescence are available. For example, some fluorescent labels exhibit a change in excitation or emission spectra, some exhibit resonance energy transfer where one fluorescent reporter loses fluorescence, while a second gains in fluorescence, some exhibit a loss (quenching) or appearance of fluorescence, while some report rotational movements.

Further, in order to obtain sufficient material for labeling, multiple amplifications may be pooled, instead of increasing the number of amplification cycles per reaction. Alternatively, labeled nucleotides can be incorporated into the last cycles of the amplification reaction. e.g., 30 cycles of PCR (no label)+10 cycles of PCR (plus label).

In particular embodiments, the disclosure provides probes that can attach to the linked DNA segments. As used herein, the term “probe” refers to a molecule (e.g., an oligonucleotide, whether occurring naturally as in a purified restriction digest or produced synthetically, recombinantly or by PCR amplification), that is capable of hybridizing to another molecule of interest (e.g., another oligonucleotide). When probes are oligonucleotides, they may be single-stranded or double-stranded. Probes are useful in the detection, identification, and isolation of particular targets (e.g., gene sequences). In some cases, the probes may be associated with a label so that is detectable in any detection system including, but not limited to, enzyme (e.g., ELISA, as well as enzyme-based histochemical assays), fluorescent, radioactive, and luminescent systems

With respect to arrays and microarrays, the term “probe” is used to refer to any hybridizable material that is affixed to the array for the purpose of detecting a nucleotide sequence that has hybridized to said probe. In some cases, the probes can about 10 bp to 500 bp, about 10 bp to 250 bp, about 20 bp to 250 bp, about 20 bp to 200 bp, about 25 bp to 200 bp, about 25 bp to 100 bp, about 30 bp to 100 bp, or about 30 bp to 80 bp. In some cases, the probes can be greater than about 10 bp, about 20 bp, about 30 bp, about 40 bp, about 50 bp, about 60 bp, about 70 bp, about 80 bp, about 90 bp, about 100 bp, about 150 bp, about 200 bp, about 250 bp, about 300 bp, about 400 bp, or about 500 bp in length. For example, the probes can be about 20 to about 50 bp in length. Examples and rationale for probe design can be found in WO95/11995, EP 717,113 and WO97/29212

The probes, array of probes or set of probes can be immobilized on a support. Supports (e.g., solid supports) can be made of a variety of materials-such as glass, silica, plastic, nylon, or nitrocellulose. Supports can be rigid and have a planar surface. Supports can have from about 1 to 10,000,000 resolved loci. For example, a support can have about 10 to 10,000,000, about 10 to 5,000,000, about 100 to 5,000,000, about 100 to 4,000,000, about 1000 to 4,000,000, about 1000 to 3,000,000, about 10,000 to 3,000,000, about 10,000 to 2,000,000, about 100,000 to 2,000,000, or about 100,000 to 1,000,000 resolved loci. The density of resolved loci can be at least about 10, about 100, about 1000, about 10,000, about 100,000 or about 1,000,000 resolved loci within a square centimeter. In some cases, each resolved locus can be occupied by >95% of a single type of oligonucleotide. In other cases, each resolved locus can be occupied by pooled mixtures of probes or a set of probes. In further cases, some resolved loci are occupied by pooled mixtures of probes or a set of probes, and other resolved loci are occupied by >95% of a single type of oligonucleotide.

In some cases, the number of probes for a given nucleotide sequence on the array can be in large excess to the DNA sample to be hybridized to such array. For example, the array can have about 10, about 100, about 1000, about 10,000, about 100,000, about 1,000,000, about 10,000,000, or about 100,000,000 times the number of probes relative to the amount of DNA in the input sample.

In some cases, an array can have about 10, about 100, about 1000, about 10,000, about 100,000, about 1,000,000, about 10,000,000, about 100,000,000, or about 1,000,000,000 probes.

Arrays of probes or sets of probes may be synthesized in a step-by-step manner on a support or can be attached in presynthesized form. One method of synthesis is VLSIPS™ (as described in U.S. Pat. No. 5,143,854 and EP 476,014), which entails the use of light to direct the synthesis of oligonucleotide probes in high-density, miniaturized arrays. Algorithms for design of masks to reduce the number of synthesis cycles are described in U.S. Pat. Nos. 5,571,639 and 5,593,839. Arrays can also be synthesized in a combinatorial fashion by delivering monomers to cells of a support by mechanically constrained flowpaths, as described in EP 624,059. Arrays can also be synthesized by spotting reagents on to a support using an ink jet printer (see, for example, EP 728.520).

In some embodiments, the present disclosure provides methods for hybridizing the linked DNA segments onto an array. A “substrate” or an “array” is an intentionally created collection of nucleic acids which can be prepared either synthetically or biosynthetically and screened for biological activity in a variety of different formats (e.g., libraries of soluble molecules; and libraries of oligonucleotides tethered to resin beads, silica chips, or other solid supports). Additionally, the term “array” includes those libraries of nucleic acids which can be prepared by spotting nucleic acids of essentially any length (e.g., from 1 to about 1000 nucleotide monomers in length) onto a substrate.

Array technology and the various associated techniques and applications are described generally in numerous textbooks and documents. For example, these include Lemieux et al., 1998. Molecular Breeding 4, 277-289; Schena and Davis, Parallel Analysis with Biological Chips, in PCR Methods Manual (eds. M. Innis. D. Gelfand, J. Sninsky); Schena and Davis, 1999. Genes, Genomes and Chips. In DNA Microarrays: A Practical Approach (ed. M. Schena). Oxford University Press. Oxford, UK, 1999); The Chipping Forecast (Nature Genetics special issue; January 1999 Supplement); Mark Schena (Ed.). Microarray Biochip Technology, (Eaton Publishing Company); Cortes, 2000, The Scientist 14[17]:25; Gwynn and Page, Microarray analysis: the next revolution in molecular biology, Science, 1999 Aug. 6; and Eakins and Chu, 1999, Trends in Biotechnology, 17, 217-218.

In general, any library may be arranged in an orderly manner into an array, by spatially separating the members of the library. Examples of suitable libraries for arraying include nucleic acid libraries (including DNA, cDNA, oligonucleotide, etc. libraries), peptide, polypeptide, and protein libraries, as well as libraries comprising any molecules, such as ligand libraries, among others.

The library can be fixed or immobilized onto a solid phase (e.g., a solid substrate), to limit diffusion and admixing of the members. In some cases, libraries of DNA binding ligands may be prepared. In particular, the libraries may be immobilized to a substantially planar solid phase, including membranes and non-porous substrates such as plastic and glass. Furthermore, the library can be arranged in such a way that indexing (i.e., reference or access to a particular member) is facilitated. In some examples, the members of the library can be applied as spots in a grid formation. Common assay systems may be adapted for this purpose. For example, an array may be immobilized on the surface of a microplate, either with multiple members in a well, or with a single member in each well. Furthermore, the solid substrate may be a membrane, such as a nitrocellulose or nylon membrane (for example, membranes used in blotting experiments). Alternative substrates include glass, or silica-based substrates. Thus, the library can be immobilized by any suitable method, for example, by charge interactions, or by chemical coupling to the walls or bottom of the wells, or the surface of the membrane. Other means of arranging and fixing may be used, for example, pipetting, drop-touch, piezoelectric means, ink-jet and bubblejet technology, electrostatic application, etc. In the case of silicon-based chips, photolithography may be utilized to arrange and fix the libraries on the chip.

The library may be arranged by being “spotted” onto the solid substrate; this may be done by hand or by making use of robotics to deposit the members. In general, arrays may be described as macroarrays or microarrays, the difference being the size of the spots. Macroarrays can contain spot sizes of about 300 microns or larger and may be easily imaged by existing gel and blot scanners. The spot sizes in microarrays can be less than 200 microns in diameter and these arrays usually contain thousands of spots. Thus, microarrays may require specialized robotics and imaging equipment, which may need to be custom made. Instrumentation is described generally in a review by Cortese, 2000. The Scientist 14[11]:26.

Techniques for producing immobilized libraries of DNA molecules have been described. Generally, most such methods describe how to synthesize single-stranded nucleic acid molecule libraries, using, for example, masking techniques to build up various permutations of sequences at the various discrete positions on the solid substrate. U.S. Pat. No. 5,837,832 describes an improved method for producing DNA arrays immobilized to silicon substrates based on very large-scale integration technology. In particular, U.S. Pat. No. 5,837,832 describes a strategy called “tiling” to synthesize specific sets of probes at spatially-defined locations on a substrate which may be used to produce the immobilized DNA libraries of the present disclosure. U.S. Pat. No. 5,837,832 also provides references for earlier techniques that may also be used. In other cases, arrays may also be built using photo deposition chemistry.

Arrays of peptides (or peptidomimetics) may also be synthesized on a surface in a manner that places each distinct library member (e.g., unique peptide sequence) at a discrete, predefined location in the array. The identity of each library member is determined by its spatial location in the array. The locations in the array where binding interactions between a predetermined molecule (e.g., a target or probe) and reactive library members occur is determined, thereby identifying the sequences of the reactive library members on the basis of spatial location. These methods are described in U.S. Pat. No. 5,143,854: WO90/15070 and WO92/10092; Fodor et al. (1991) Science, 251: 767; Dower and Fodor (1991) Ann. Rep. Med. Chem., 26: 271

To aid detection, labels can be used (as discussed above-such as any readily detectable reporter, for example, a fluorescent, bioluminescent, phosphorescent, radioactive, etc. reporter. Such reporters, their detection, coupling to targets/probes, etc. are discussed elsewhere in this document. Labelling of probes and targets is also disclosed in Shalon et al., 1996, Genome Res 6(7):639-45.

Examples of some commercially available microarray formats are set out in Marshall and Hodgson, 1998, Nature Biotechnology, 16(1), 27-31.

In order to generate data from array-based assays a signal can be detected to signify the presence of or absence of hybridization between a probe and a nucleotide sequence. Further, direct and indirect labeling techniques can also be utilized. For example, direct labeling incorporates fluorescent dyes directly into the nucleotide sequences that hybridize to the array associated probes (e.g., dyes are incorporated into nucleotide sequence by enzymatic synthesis in the presence of labeled nucleotides or PCR primers). Direct labeling schemes can yield strong hybridization signals, for example, by using families of fluorescent dyes with similar chemical structures and characteristics, and can be simple to implement. In cases comprising direct labeling of nucleic acids, cyanine or alexa analogs can be utilized in multiple-fluor comparative array analyses. In other embodiments, indirect labeling schemes can be utilized to incorporate epitopes into the nucleic acids either prior to or after hybridization to the microarray probes. One or more staining procedures and reagents can be used to label the hybridized complex (e.g., a fluorescent molecule that binds to the epitopes, thereby providing a fluorescent signal by virtue of the conjugation of dye molecule to the epitope of the hybridized species).

### Sequencing

In various embodiments, suitable sequencing methods described herein or otherwise known will be used to obtain sequence information from nucleic acid molecules within a sample. Sequencing can be accomplished through classic Sanger sequencing methods. Sequence can also be accomplished using high-throughput systems some of which allow detection of a sequenced nucleotide immediately after or upon its incorporation into a growing strand, i.e., detection of sequence in real time or substantially real time. In some cases, high-throughput sequencing generates at least 1,000, at least 5,000, at least 10,000, at least 20,000, at least 30,000, at least 40,000, at least 50,000, at least 100,000 or at least 500,000 sequence reads per hour; where the sequencing reads can be at least about 50, about 60, about 70, about 80, about 90, about 100, about 120, about 150, about 180, about 210, about 240, about 270, about 300, about 350, about 400, about 450, about 500, about 600, about 700, about 800, about 900, or about 1000 bases per read. In some cases, the sequencing reads can be at least about 5 kb, about 8 kb, about 10 kb, about 12 kb, about 15 kb, about 20 kb, about 25 kb, about 30 kb, about 35 kb, about 40 kb, about 45 kb, about 50 kb, about 60 kb, about 70 kb, about 80 kb, about 90 kb, about 100 kb, about 120 kb, about 150 kb, or more per read.

Sequencing can be whole-genome, with or without enrichment of particular regions of interest. Sequencing can be targeted to particular regions of the genome. Regions of the genome that can be enriched for or targeted include but are not limited to single genes (or regions thereof), gene panels, gene fusions, human leukocyte antigen (HLA) loci (e.g., Class I HLA-A, B, and C; Class II HLA-DRB1/3/4/5, HLA-DQA1, HLA-DQB1, HLA-DPA1, HLA-DPB1), exonic regions, exome, and other loci. Genomic regions can be relevant to immune response, immune repertoire, immune cell diversity, transcription (e.g., exome), cancers (e.g., BRCA1. BRCA2, panels of genes or regions thereof such as hotspot regions, somatic variants. SNVs, amplifications, fusions, tumor mutational burden (TMB), microsatellite instability (MSI)), cardiac diseases, inherited diseases, and other diseases or conditions. A variety of methods can be used to enrich for or target regions of interest, including but not limited to sequence capture. In some cases. Capture Hi-C(CHi-C) or CHi-C-like protocols are employed, employing a sequence capture step (e.g., by target enrichment array) before or after library preparation.

In some embodiments, high-throughput sequencing involves the use of technology available by Illumina's Genome Analyzer IIX, MiSeq personal sequencer, or HiSeq systems, such as those using HiSeq 2500, HiSeq 1500, HiSeq 2000, or HiSeq 100) machines. These machines use reversible terminator-based sequencing by synthesis chemistry. These machines can do 200 billion DNA reads or more in eight days. Smaller systems may be utilized for runs within 3, 2, 1 days or less time.

In some embodiments, high-throughput sequencing involves the use of technology available by ABI Solid System. This genetic analysis platform that enables massively parallel sequencing of clonally-amplified DNA fragments linked to beads. The sequencing methodology is based on sequential ligation with dye-labeled oligonucleotides.

The next generation sequencing can comprise ion semiconductor sequencing (e.g., using technology from Life Technologies (Ion Torrent)). Ion semiconductor sequencing can take advantage of the fact that when a nucleotide is incorporated into a strand of DNA, an ion can be released. To perform ion semiconductor sequencing, a high-density array of micromachined wells can be formed. Each well can hold a single DNA template. Beneath the well can be an ion sensitive layer, and beneath the ion sensitive layer can be an ion sensor. When a nucleotide is added to a DNA. H+ can be released, which can be measured as a change in pH. The H+ ion can be converted to voltage and recorded by the semiconductor sensor. An array chip can be sequentially flooded with one nucleotide after another. No scanning, light, or cameras can be required. In some cases, an IONPROTON™ Sequencer is used to sequence nucleic acid. In some cases, an IONPGM™ Sequencer is used. The Ion Torrent Personal Genome Machine (PGM). The PGM can do 10 million reads in two hours.

In some embodiments, high-throughput sequencing involves the use of technology available by Helicos BioSciences Corporation (Cambridge, Massachusetts) such as the Single Molecule Sequencing by Synthesis (SMSS) method. SMSS is unique because it allows for sequencing the entire human genome in up to 24 hours. Finally, SMSS is described in part in US Publication Application Nos. 20060024711; 20060024678; 20060012793; 20060012784; and 20050100932.

In some embodiments, high-throughput sequencing involves the use of technology available by 454 Lifesciences. Inc. (Branford, Connecticut) such as the PicoTiterPlate device which includes a fiber optic plate that transmits chemiluminescent signal generated by the sequencing reaction to be recorded by a CCD camera in the instrument. This use of fiber optics allows for the detection of a minimum of 20 million base pairs in 4.5 hours.

Methods for using bead amplification followed by fiber optics detection are described in Marguiles. M., et al. “Genome sequencing in microfabricated high-density pricolitre reactors,” Nature, doi:10.1038/nature03959; and well as in US Publication Application Nos. 20020012930; 20030068629; 20030100102; 20030148344; 20040248161; 20050079510, 20050124022; and 20060078909.

In some embodiments, high-throughput sequencing is performed using Clonal Single Molecule Array (Solexa, Inc.) or sequencing-by-synthesis (SBS) utilizing reversible terminator chemistry. These technologies are described in part in U.S. Pat. Nos. 6,969,488; 6,897,023; 6,833,246; 6,787,308; and US Publication Application Nos. 20040106110; 20030064398; 20030022207; and Constans, A., The Scientist 2003, 17(13):36.

The next generation sequencing technique can comprise real-time (SMRT™) technology by Pacific Biosciences. In SMRT, each of four DNA bases can be attached to one of four different fluorescent dyes. These dyes can be phospho linked. A single DNA polymerase can be immobilized with a single molecule of template single stranded DNA at the bottom of a zero-mode waveguide (ZMW). A ZMW can be a confinement structure which enables observation of incorporation of a single nucleotide by DNA polymerase against the background of fluorescent nucleotides that can rapidly diffuse in an out of the ZMW (in microseconds). It can take several milliseconds to incorporate a nucleotide into a growing strand. During this time, the fluorescent label can be excited and produce a fluorescent signal, and the fluorescent tag can be cleaved off. The ZMW can be illuminated from below. Attenuated light from an excitation beam can penetrate the lower 20-30 nm of each ZMW. A microscope with a detection limit of 20 zepto liters (20×10⁻²¹ liters) can be created. The tiny detection volume can provide 1000-fold improvement in the reduction of background noise. Detection of the corresponding fluorescence of the dye can indicate which base was incorporated. The process can be repeated.

In some cases, the next generation sequencing is nanopore sequencing (see, e.g., Soni G V and Meller A. (2007) Clin Chem 53: 1996-2001). A nanopore can be a small hole, of the order of about one nanometer in diameter. Immersion of a nanopore in a conducting fluid and application of a potential across it can result in a slight electrical current due to conduction of ions through the nanopore. The amount of current which flows can be sensitive to the size of the nanopore. As a DNA molecule passes through a nanopore, each nucleotide on the DNA molecule can obstruct the nanopore to a different degree. Thus, the change in the current passing through the nanopore as the DNA molecule passes through the nanopore can represent a reading of the DNA sequence. The nanopore sequencing technology can be from Oxford Nanopore Technologies; e.g., a GridlON system. A single nanopore can be inserted in a polymer membrane across the top of a microwell. Each microwell can have an electrode for individual sensing. The microwells can be fabricated into an array chip, with 100,000 or more microwells (e.g., more than 200,000, 300,000, 400,000, 500,000, 600,000, 700,000, 800,000, 900,000, or 1,000,000) per chip. An instrument (or node) can be used to analyze the chip. Data can be analyzed in real-time. One or more instruments can be operated at a time. The nanopore can be a protein nanopore, e.g., the protein alpha-hemolysin, a heptameric protein pore. The nanopore can be a solid-state nanopore made, e.g., a nanometer sized hole formed in a synthetic membrane (e.g., SiNx, or SiO2). The nanopore can be a hybrid pore (e.g., an integration of a protein pore into a solid-state membrane). The nanopore can be a nanopore with an integrated sensor (e.g., tunneling electrode detectors, capacitive detectors, or graphene based nano-gap or edge state detectors (see e.g., Garaj et al. (2010) Nature vol. 67, doi: 10.1038/nature09379)). A nanopore can be functionalized for analyzing a specific type of molecule (e.g., DNA, RNA, or protein). Nanopore sequencing can comprise “strand sequencing” in which intact DNA polymers can be passed through a protein nanopore with sequencing in real time as the DNA translocates the pore. An enzyme can separate strands of a double stranded DNA and feed a strand through a nanopore. The DNA can have a hairpin at one end, and the system can read both strands. In some cases, nanopore sequencing is “exonuclease sequencing” in which individual nucleotides can be cleaved from a DNA strand by a processive exonuclease, and the nucleotides can be passed through a protein nanopore. The nucleotides can transiently bind to a molecule in the pore (e.g., cyclodextran). A characteristic disruption in current can be used to identify bases.

Nanopore sequencing technology from GENIA can be used. An engineered protein pore can be embedded in a lipid bilayer membrane. “Active Control” technology can be used to enable efficient nanopore-membrane assembly and control of DNA movement through the channel. In some cases, the nanopore sequencing technology is from NABsys. Genomic DNA can be fragmented into strands of average length of about 100 kb. The 100 kb fragments can be made single stranded and subsequently hybridized with a 6-mer probe. The genomic fragments with probes can be driven through a nanopore, which can create a current-versus-time tracing. The current tracing can provide the positions of the probes on each genomic fragment. The genomic fragments can be lined up to create a probe map for the genome. The process can be done in parallel for a library of probes. A genome-length probe map for each probe can be generated. Errors can be fixed with a process termed “moving window Sequencing by Hybridization (mwSBH).” In some cases, the nanopore sequencing technology is from IBM/Roche. An electron beam can be used to make a nanopore sized opening in a microchip. An electrical field can be used to pull or thread DNA through the nanopore. A DNA transistor device in the nanopore can comprise alternating nanometer sized layers of metal and dielectric. Discrete charges in the DNA backbone can get trapped by electrical fields inside the DNA nanopore. Turning off and on gate voltages can allow the DNA sequence to be read.

The next generation sequencing can comprise DNA nanoball sequencing (as performed, e.g., by Complete Genomics; see e.g., Drmanac et al. (2010) Science 327: 78-81). DNA can be isolated, fragmented, and size selected. For example, DNA can be fragmented (e.g., by sonication) to a mean length of about 500 bp. Adaptors (Adl) can be attached to the ends of the fragments. The adaptors can be used to hybridize to anchors for sequencing reactions. DNA with adaptors bound to each end can be PCR amplified. The adaptor sequences can be modified so that complementary single strand ends bind to each other forming circular DNA. The DNA can be methylated to protect it from cleavage by a type IIS restriction enzyme used in a subsequent step. An adaptor (e.g., the right adaptor) can have a restriction recognition site, and the restriction recognition site can remain non-methylated. The non-methylated restriction recognition site in the adaptor can be recognized by a restriction enzyme (e.g., Acul), and the DNA can be cleaved by Acul 13 bp to the right of the right adaptor to form linear double stranded DNA. A second round of right and left adaptors (Ad2) can be ligated onto either end of the linear DNA, and all DNA with both adaptors bound can be PCR amplified (e.g., by PCR). Ad2 sequences can be modified to allow them to bind each other and form circular DNA. The DNA can be methylated, but a restriction enzyme recognition site can remain non-methylated on the left Adl adaptor. A restriction enzyme (e.g., Acul) can be applied, and the DNA can be cleaved 13 bp to the left of the Adl to form a linear DNA fragment. A third round of right and left adaptor (Ad3) can be ligated to the right and left flank of the linear DNA, and the resulting fragment can be PCR amplified. The adaptors can be modified so that they can bind to each other and form circular DNA. A type III restriction enzyme (e.g., EcoP15) can be added. EcoP15 can cleave the DNA 26 bp to the left of Ad3 and 26 bp to the right of Ad2. This cleavage can remove a large segment of DNA and linearize the DNA once again. A fourth round of right and left adaptors (Ad4) can be ligated to the DNA, the DNA can be amplified (e.g., by PCR), and modified so that they bind each other and form the completed circular DNA template.

Rolling circle replication (e.g., using Phi 29 DNA polymerase) can be used to amplify small fragments of DNA. The four adaptor sequences can contain palindromic sequences that can hybridize, and a single strand can fold onto itself to form a DNA nanoball (DNB™) which can be approximately 200-300 nanometers in diameter on average. A DNA nanoball can be attached (e.g., by adsorption) to a microarray (sequencing flowcell). The flow cell can be a silicon wafer coated with silicon dioxide, titanium and hexamehtyldisilazane (HMDS) and a photoresist material. Sequencing can be performed by unchained sequencing by ligating fluorescent probes to the DNA. The color of the fluorescence of an interrogated position can be visualized by a high-resolution camera. The identity of nucleotide sequences between adaptor sequences can be determined.

In some embodiments, high-throughput sequencing can take place using AnyDot.chips (Genovoxx, Germany). In particular, the AnyDot.chips allow for 10×-50× enhancement of nucleotide fluorescence signal detection. AnyDot.chips and methods for using them are described in part in International Publication Application Nos. WO 02088382. WO 03020968, WO 03031947, WO 2005044836, PCT/EP 05/05657, PCT/EP 05/05655; and German Patent Application Nos. DE 101 49 786, DE 102 14 395, DE 103 56 837, DE 10 2004 009 704, DE 10 2004 025 696, DE 10 2004 025 746, DE 10 2004 025 694, DE 10 2004 025 695, DE 10 2004 025 744, DE 10 2004 025 745, and DE 10 2005 012 301.

Other high-throughput sequencing systems include those disclosed in Venter. J., et al. Science 16 Feb. 2001; Adams, M. et al. Science 24 Mar. 2000; and M. J. Levene, et al. Science 299:682-686. January 2003; as well as US Publication Application No. 20030044781 and 2006/0078937. Overall such systems involve sequencing a target nucleic acid molecule having a plurality of bases by the temporal addition of bases via a polymerization reaction that is measured on a molecule of nucleic acid, i.e., the activity of a nucleic acid polymerizing enzyme on the template nucleic acid molecule to be sequenced is followed in real time. Sequence can then be deduced by identifying which base is being incorporated into the growing complementary strand of the target nucleic acid by the catalytic activity of the nucleic acid polymerizing enzyme at each step in the sequence of base additions. A polymerase on the target nucleic acid molecule complex is provided in a position suitable to move along the target nucleic acid molecule and extend the oligonucleotide primer at an active site. A plurality of labeled types of nucleotide analogs are provided proximate to the active site, with each distinguishable type of nucleotide analog being complementary to a different nucleotide in the target nucleic acid sequence. The growing nucleic acid strand is extended by using the polymerase to add a nucleotide analog to the nucleic acid strand at the active site, where the nucleotide analog being added is complementary to the nucleotide of the target nucleic acid at the active site. The nucleotide analog added to the oligonucleotide primer as a result of the polymerizing step is identified. The steps of providing labeled nucleotide analogs, polymerizing the growing nucleic acid strand, and identifying the added nucleotide analog are repeated so that the nucleic acid strand is further extended, and the sequence of the target nucleic acid is determined.

### Kits

In particular embodiments, the present disclosure further provides kits comprising one or more components of the disclosure. The kits can be used for any suitable application, including, without limitation, those described above. The kits can comprise, for example, a plurality of association molecules, a fixative agent, a nuclease, a ligase, and/or a combination thereof. In some cases, the association molecules can be proteins including, for example, histones. In some cases, the fixative agent can be formaldehyde or any other DNA crosslinking agent, including DSG, EGS, or DSS.

In some cases, the kit can further comprise a plurality of beads. The beads can be paramagnetic and/or are coated with a capturing agent. For example, the beads can be coated with streptavidin and/or an antibody.

In some cases, the kit can comprise adaptor oligonucleotides and/or sequencing primers. Further, the kit can comprise a device capable of amplifying the read-pairs using the adaptor oligonucleotides and/or sequencing primers.

In some cases, the kit can also comprise other reagents including, but not limited to, lysis buffers, ligation reagents (e.g., dNTPs, polymerase, polynucleotide kinase, and/or ligase buffer, etc.), and PCR reagents (e.g., dNTPs, polymerase, and/or PCR buffer, etc.),

The kit can also include instructions for using the components of the kit and/or for generating the read-pairs.

### Computers and Systems

The computer system 500 illustrated in FIG. 8 may be understood as a logical apparatus that can read instructions from media 511 and/or a network port 505, which can optionally be connected to server 509 having fixed media 512. The system, such as shown in FIG. 8 can include a CPU 501, disk drives 503, optional input devices such as keyboard 515 and/or mouse 516 and optional monitor 507. Data communication can be achieved through the indicated communication medium to a server at a local or a remote location. The communication medium can include any means of transmitting and/or receiving data. For example, the communication medium can be a network connection, a wireless connection, or an internet connection. Such a connection can provide for communication over the World Wide Web. It is envisioned that data relating to the present disclosure can be transmitted over such networks or connections for reception and/or review by a party 522 as illustrated in FIG. 8.

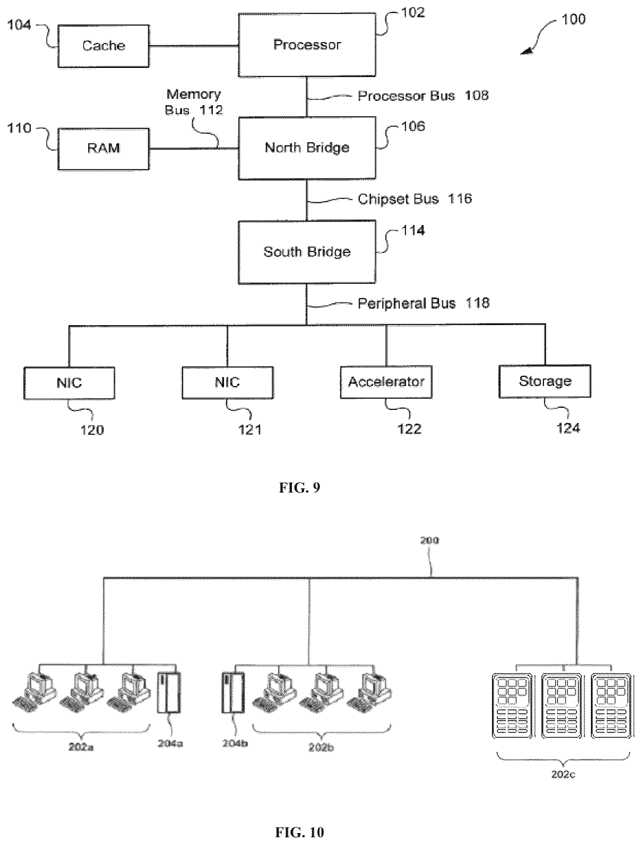

FIG. 9 is a block diagram illustrating a first example architecture of a computer system 100 that can be used in connection with example embodiments of the present disclosure. As depicted in FIG. 9, the example computer system can include a processor 102 for processing instructions. Non-limiting examples of processors include: Intel Xeon™ processor. AMD Opteron™ processor, Samsung 32-bit RISC ARM 1176JZ(F)-S v 1.0™ processor, ARM Cortex-A8 Samsung S5PC100™ processor, ARM Cortex-A8 Apple A4™ processor, Marvell PXA 930™ processor, or a functionally-equivalent processor.

Multiple threads of execution can be used for parallel processing. In some embodiments, multiple processors or processors with multiple cores can also be used, whether in a single computer system, in a cluster, or distributed across systems over a network comprising a plurality of computers, cell phones, and/or personal data assistant devices.

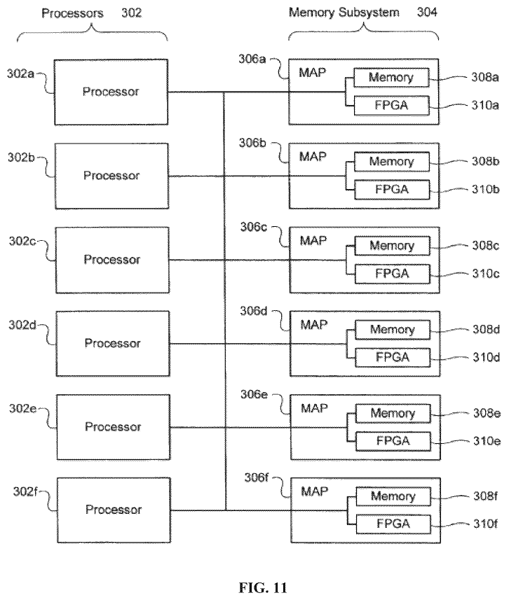

As illustrated in FIG. 10, a high-speed cache 104 can be connected to, or incorporated in, the processor 102 to provide a high-speed memory for instructions or data that have been recently, or are frequently, used by processor 102. The processor 102 is connected to a north bridge 106 by a processor bus 108. The north bridge 106 is connected to random access memory (RAM) 110 by a memory bus 112 and manages access to the RAM 110 by the processor 102. The north bridge 106 is also connected to a south bridge 114 by a chipset bus 116. The south bridge 114 is, in turn, connected to a peripheral bus 118. The peripheral bus can be, for example, PCI, PCI-X. PCI Express, or other peripheral bus. The north bridge and south bridge are often referred to as a processor chipset and manage data transfer between the processor, RAM, and peripheral components on the peripheral bus 118. In some alternative architectures, the functionality of the north bridge can be incorporated into the processor instead of using a separate north bridge chip.

In some embodiments, system 100 can include an accelerator card 122 attached to the peripheral bus 118. The accelerator can include field programmable gate arrays (FPGAs) or other hardware for accelerating certain processing. For example, an accelerator can be used for adaptive data restructuring or to evaluate algebraic expressions used in extended set processing.

Software and data are stored in external storage 124 and can be loaded into RAM 110 and/or cache 104 for use by the processor. The system 100 includes an operating system for managing system resources; non-limiting examples of operating systems include: Linux, Windows™, MACOS™, BlackBerry OS™, iOS™, and other functionally-equivalent operating systems, as well as application software running on top of the operating system for managing data storage and optimization in accordance with example embodiments of the present disclosure.

In this example, system 100 also includes network interface cards (NICs) 120 and 121 connected to the peripheral bus for providing network interfaces to external storage, such as Network Attached Storage (NAS) and other computer systems that can be used for distributed parallel processing.

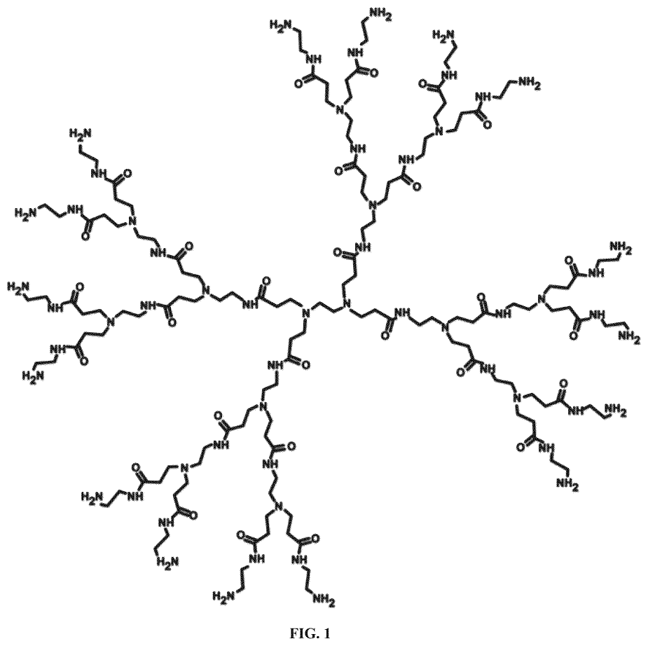

FIG. 11 is a diagram showing a network 200 with a plurality of computer systems 202a, and 202b, a plurality of cell phones and personal data assistants 202c, and Network Attached Storage (NAS) 204a, and 204b. In example embodiments, systems 202a, 202b, and 202c can manage data storage and optimize data access for data stored in Network Attached Storage (NAS) 204a and 204b. A mathematical model can be used for the data and be evaluated using distributed parallel processing across computer systems 202a, and 202b, and cell phone and personal data assistant systems 202c. Computer systems 202a, and 202b, and cell phone and personal data assistant systems 202c can also provide parallel processing for adaptive data restructuring of the data stored in Network Attached Storage (NAS) 204a and 204b. FIG. 1I illustrates an example only, and a wide variety of other computer architectures and systems can be used in conjunction with the various embodiments of the present disclosure. For example, a blade server can be used to provide parallel processing. Processor blades can be connected through a back plane to provide parallel processing. Storage can also be connected to the back plane or as Network Attached Storage (NAS) through a separate network interface.

In some example embodiments, processors can maintain separate memory spaces and transmit data through network interfaces, back plane, or other connectors for parallel processing by other processors. In other embodiments, some or all of the processors can use a shared virtual address memory space.

FIG. 12 is a block diagram of a multiprocessor computer system 300 using a shared virtual address memory space in accordance with an example embodiment. The system includes a plurality of processors 302a-f that can access a shared memory subsystem 304. The system incorporates a plurality of programmable hardware memory algorithm processors (MAPs) 306a-f in the memory subsystem 304. Each MAP 306a-f can comprise a memory 308a-f and one or more field programmable gate arrays (FPGAs) 310a-f. The MAP provides a configurable functional unit and particular algorithms, or portions of algorithms, can be provided to the FPGAs 310a-f for processing in close coordination with a respective processor. For example, the MAPs can be used to evaluate algebraic expressions regarding the data model and to perform adaptive data restructuring in example embodiments. In this example, each MAP is globally accessible by all of the processors for these purposes. In one configuration, each MAP can use Direct Memory Access (DMA) to access an associated memory 308a-f, allowing it to execute tasks independently of, and asynchronously from, the respective microprocessor 302a-f. In this configuration, a MAP can feed results directly to another MAP for pipelining and parallel execution of algorithms.

The above computer architectures and systems are examples only, and a wide variety of other computer, cell phone, and personal data assistant architectures and systems can be used in connection with example embodiments, including systems using any combination of general processors, co-processors. FPGAs and other programmable logic devices, system on chips (SOCs), application specific integrated circuits (ASICs), and other processing and logic elements. In some embodiments, all or part of the computer system can be implemented in software or hardware. Any variety of data storage media can be used in connection with example embodiments, including random access memory, hard drives, flash memory, tape drives, disk arrays. Network Attached Storage (NAS) and other local or distributed data storage devices and systems.

In example embodiments, the computer system can be implemented using software modules executing on any of the above or other computer architectures and systems. In other embodiments, the functions of the system can be implemented partially or completely in firmware, programmable logic devices such as field programmable gate arrays (FPGAs), system on chips (SOCs), application specific integrated circuits (ASICs), or other processing and logic elements. For example, the Set Processor and Optimizer can be implemented with hardware acceleration through the use of a hardware accelerator card, such as accelerator card 122 illustrated in FIG. 10.

### Definitions

Unless defined otherwise, all technical and scientific terms used herein have the same meaning as commonly understood to one of ordinary skill in the art to which this disclosure belongs. Although any methods and reagents similar or equivalent to those described herein can be used in the practice of the disclosed methods and compositions, the exemplary methods and materials are now described.

As used herein and in the appended claims, the singular forms “a,” “and,” and “the” include plural referents unless the context clearly dictates otherwise. Thus, for example, reference to “contig” includes a plurality of such contigs and reference to “probing the physical layout of chromosomes” includes reference to one or more methods for probing the physical layout of chromosomes and equivalents thereof known to those skilled in the art, and so forth.

Also, the use of “and” means “and/or” unless stated otherwise. Similarly, “comprise,” “comprises,” “comprising,” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

The term “sequencing read” as used herein, refers to a fragment of DNA in which the sequence has been determined.

The term “contigs” as used herein, refers to contiguous regions of DNA sequence. “Contigs” can be determined by any number methods known in the art, such as, by comparing sequencing reads for overlapping sequences, and/or by comparing sequencing reads against a database of known sequences in order to identify which sequencing reads have a high probability of being contiguous.

The term “subject” as used herein can refer to any eukaryotic or prokaryotic organism.

The term “stabilized” as used herein can describe a sample that has been preserved or otherwise protected from degradation. In some cases, a stabilized sample is crosslinked or treated with a fixative or crosslinking agent. In some cases, a stabilized sample is treated with formaldehyde, formalin, paraformaldehyde, glutaraldehyde, osmium tetroxide, or the like.

The term “about” as used herein can describe a number, unless otherwise specified, as a range of values including that number plus or minus 10% of that number.

As used herein, the term “about” a number refers to a range spanning+/−10% of that number, while “about” a range refers to 10% lower than a stated range limit spanning to 10% greater than a stated range limit.

As used herein, a sequence segment on a linker or otherwise is partition designating, or cell designating when identification of its sequence facilitates assigning adjacent nucleic acid sequence to a particular first partition or cell of origin to the exclusion of a second partition or cell of origin. A distinguishing sequence is in some cases unique to a partition or cell, such that it distinguishes from all other cells, and when this is technically feasible, unique tags facilitate downstream analysis. However, unique sequence is not in all cases required. In some cases, redundant barcoding is resolved computationally downstream, such that a tag that is not unique is nonetheless sufficient to distinguish nucleic acids of a first partition or cell from a second partition or cell.

As used herein, a cluster is a region of a nucleic acid reference to which a plurality of distinct end adjacent sequences or sequence tags map. In some cases, the proximity of one region to a second region is assessed at least in part by counting the number of cluster constituents of a first cluster that co-occur in paired end reads with cluster constituents of a second cluster.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Creation of Long Concatemers Using Dendrimer Binding and Proximity Ligation

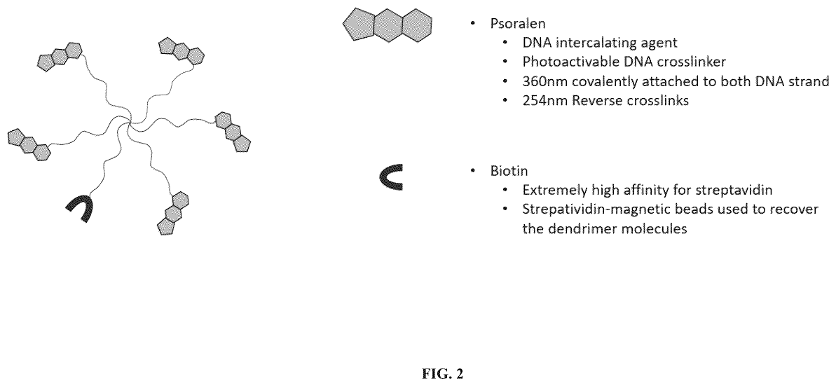

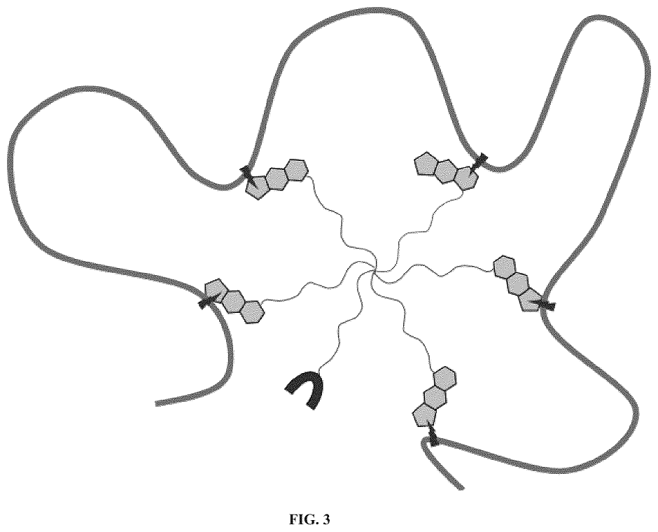

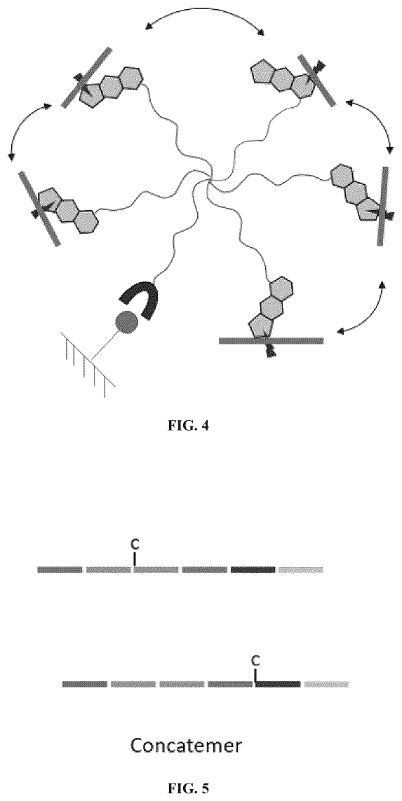

A polyamidoamine (PAMAM) dendrimer (FIG. 1) was coupled with psoralen to bind DNA and biotin to allow purification of dendrimers bound to DNA (FIG. 2). Psoralen was reacted with chromatin to capture proximal DNA (FIG. 3). The DNA bound to the dendrimers was fragmented using an endonuclease and the bound fragments purified using C1 beads. The purified fragments on the dendrimer were ligated to each other to create a concatemer and the concatemer was purified from the dendrimer (FIG. 4).

In some cases inter-DNA ligation is mediated using adapters carrying a dibenzocyclooctyne-amine (DBCON) added to the steps above. A photocleavable biotin-azide is bound to the adapter using click chemistry. Streptavidin-magnetic beads is used to purify the resulting concatemer and 360 nm UV light liberates the DNA from the biotin (FIG. 5).

### Example 2: Proximity Ligation of Dendrimer-Bound Nucleic Acids

A 50 kb naked DNA from bacteriophage lambda was bound with a PAMAM dendrimer having psoralen DNA binding moieties. The complexed DNA was digested with HincII resulting in about 40 bacteriophage lambda DNA fragments bound to the dendrimer. The complexed fragments were then ligated to each other, and the ligated DNA fragments were isolated from the dendrimer and sequenced.

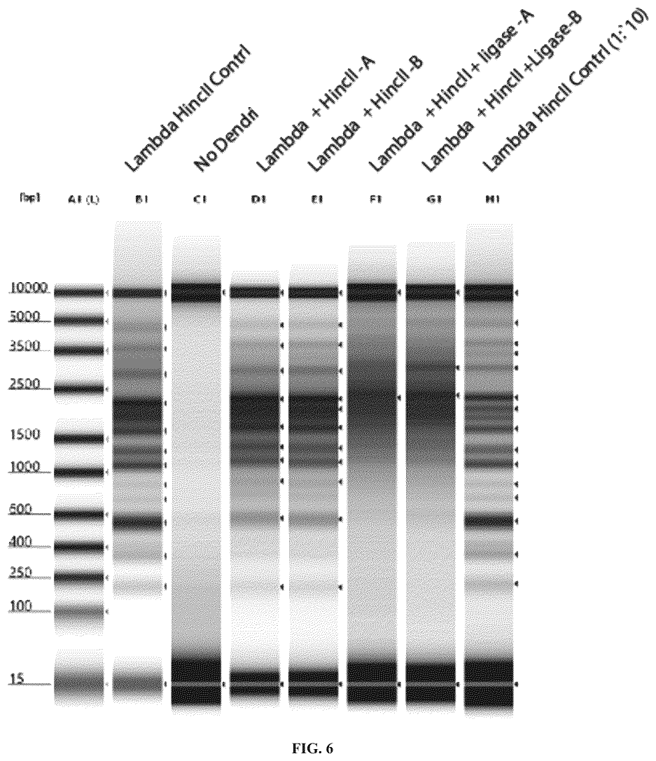

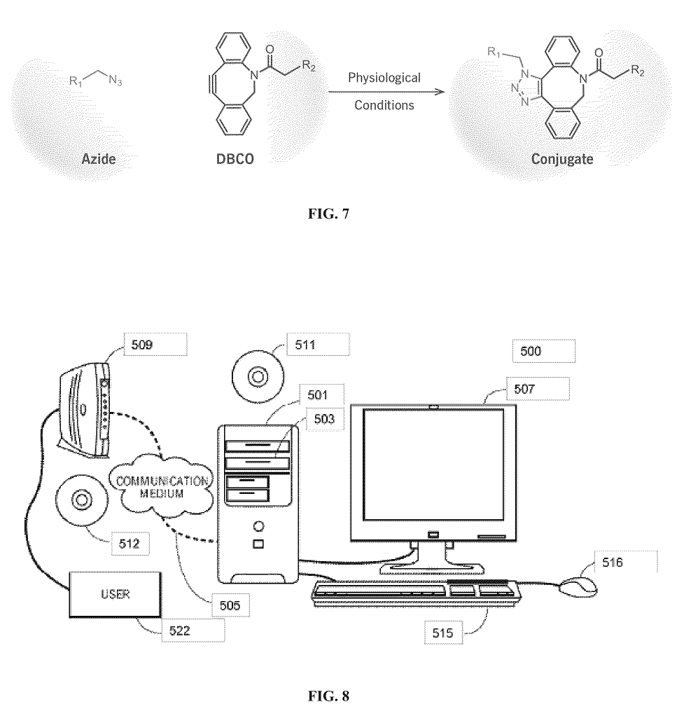

FIG. 6 shows a DNA electrophoresis analysis of various steps in the process, where digestion of the lambda DNA with HincII yields a ladder of fragments. Adding ligase to the digested fragments yields a large fragment and less DNA in the ladder of smaller fragments. From left to right, lanes A 1 through H1 contain: ladder (A1); lambda DNA digested with HincII as a control (B1); results of the protocol conducted with no dendrimer (C1); results of the protocol conducted without ligase (two replicates, D1 and E1); results of the protocol conducted with ligase (two replicates, F1 and G1); lambda DNA digested with HincII as a control at 1:10 ratio (H1).

### Example 3: Single Cell Topology Analysis

Dendrimers are coupled to a solid surface with a cleavable linker such as carboxylic beads bound to disulfided-amide-carboxyl and carboxyl linked to an amine dendrimer. A small number of biotin molecules (NHS-biotin) are coupled to each dendrimer. A constant sequence oligonucleotide is coupled to each dendrimer using amine-amine bonds. A unique barcode (UMI) is bound to each dendrimer using a split-pool approach by repeating the following steps: solid-phase bound oligonucleotide containing dendrimer is split into four tubes; a single 3′ OH block nucleotide, one different nucleotide per pool is added using terminal transferase; the four tubes of dendrimers are pooled into a single tube; and the 3′ OH block is cleaved from the nucleotides before splitting (per the first step). The UMI-tagged dendrimers are released in solution and the dendrimers are treated with an oligonucleotide complementary to the constant sequence that creates a 5′ T overhang on each arm of the dendrimer.

The cells or tissue are fixed using formaldehyde and disuccinimidyl glutarate. Nuclei are obtained from the fixed cells are tissue using NP-40 in low salt conditions. The nuclei are then treated with micrococcal nuclease (MNase) to create single nucleosomes in each nucleus. The resulting DNA ends are repaired to make blunt ends and extended with an untemplated A with Taq polymerase.

The dendrimers and the nuclei are then mixed with T4 DNA ligase such that each UMI-dendrimer will barcode DNA in the same proximity within each nucleus.

Dendrimer tagged cells are then given a cell-specific UMI using a split-pool approach as above by repeating the following steps: dendrimer containing nuclei are split into four tubes; a single 3′ OH blocked nucleotide, one different nucleotide per tube, using terminal transferase; the four tubes of dendrimers are pooled into a single tube and the 3′ OH block is cleaved from the nucleotides before splitting (per the first step). The crosslinking is reversed and the samples are treated with proteinase K to release the dendrimers into solution. The dendrimers are captured onto streptavidin beads. A 3′ sequencing primer is added by ligating to the pre-adenylated oligonucleotide and a 5′ sequencing adapter is added by ligation. The nucleic acid in the samples is amplified by PCR and the samples are sequenced. Resulting sequences contain a constant sequence, a dendrimer-specific UMI, a sample sequence, and a cell/nucleus-specific UMI.

### Example 4: Aggregation of Concatemerization of Cell-Free DNA for Long-Read Sequencing

A solution of G5 PAMAM dendrimer with psoralen DNA binding moieties and a biotin tag is mixed with a serum sample comprising cell-free deoxyribonucleic acids (cfDNA). The cfDNA in the sample binds to the psoralen on the dendrimer. Ends of the cfDNA are repaired to make blunt ends and the cfDNA fragments are ligated to each other to create concatemers of cfDNA fragments bound to the same dendrimer. The concatemers are released from the dendrimer and prepared for sequencing.

### Example 5: Barcode-Dendrimer Analysis

A sample of cells or nuclei is crosslinked then nucleic acids in the cells or nuclei are digested with MNase. Nucleic acid ends are repaired to make blunt ends. The sample is then contacted with dendrimers that are preloaded with oligonucleotides that have a barcode and a constant sequence. MNase digested nucleic acids are ligated to the oligonucleotides. Dendrimers are isolated and barcoded fragments are retrieved from the dendrimers and sequenced. Sequences are associated as being proximate to each other based on a shared barcode from the same dendrimer. Higher order information is obtained compared with proximity ligation because knowledge of 3, 4, 5, 6, or more fragments near each other is obtained rather than just pairs of connections.

### Example 6: Microbiome Analysis

A microbiome sample is obtained and cells from the sample are crosslinked. Samples are contacted to a dendrimer; then nucleic acids are digested with MNase to generate fragments. Fragments are ligated to create concatemers. The concatemers are isolated and sequenced. Variants are assigned to different organisms based on how they associate in the concatemers. Alternatively, nucleic acids are digested with MNase prior to contact with a barcoded dendrimer. Variants are assigned to different organisms based on how they associate with a barcode of the dendrimer.

### Example 7: Spatial Genomics

Barcoded dendrimers are bound to digested nucleic acid molecules in a tissue section slide. On slide sequencing creates a map of where each dendrimer is bound on the tissue section. Dendrimers and attached sample nucleic acid fragments are isolated. Sequences are obtained from each barcode and associated nucleic acids. The information is combined and sequences in each area of the tissue section are determined. The resolution of this information is tuned by the size of the dendrimer where smaller dendrimers allow for smaller resolution compared with larger dendrimers.

### Example 8: Proximity Ligation Method with Psoralen Dendrimers

A cell sample is prepared for sequencing using proximity ligation. The cells in the sample are crosslinked using formaldehyde and disuccinimidyl glutarate. Nuclei from the cells are isolated by treatment with a Triton X solution. A psoralen dendrimer, created by mixing NHS-ester-psoralen mixed with PAMAM dendrimers to terminate each polymer with a primary amine which reacts at neutral pH to conjugate the psoralen molecules to the dendrimers, is added to the nuclei and photoactivated with 360 nm light. Nuclei are digested with DNase and the ends are fixed for T/A ligation. An adapter is ligated to the ends. Excess adapters are washed away. The adapters are phosphorylated prior to conducting proximity ligation. The psoralen cross-link is reversed with hot alkali treatment ore UV radiation at 254 nm. DNA is purified from the mixture and sequenced.

The method creates concatemers, maintaining multiple DNA fragments in space with a confined region with more cross-linking events thereby forming multiple junctions.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

What is claimed is:

1. A composition comprising: (a) a dendrimer comprising a plurality of nucleic acid binding moieties; and (b) a plurality of nucleic acid fragments.

2. The composition of claim 1, wherein said plurality of nucleic acid fragments are derived from a common chromosome.

3. The composition of claim 1, wherein said plurality of nucleic acid fragments are derived from different chromosomes.

4. The composition of claim 1, wherein said plurality of nucleic acid fragments comprise cell-free nucleic acids.

5. The composition of claim 1, wherein said plurality of nucleic acid fragments are proximal to each other in a cell.

6. The composition of any one of claims 1 to 5, wherein said dendrimer comprises poly(amidoamine) (PAMAM).

7. The composition of any one of claims 1 to 6, wherein said dendrimer is about 3.2 kDa to about 116 kDa.

8. The composition of any one of claims 1 to 7, wherein said dendrimer comprises about 16 to about 512 nucleic acid binding moieties.

9. The composition of any one of claims 1 to 8, wherein said plurality of nucleic acid binding moieties comprises a DNA intercalating agent.

10. The composition of any one of claims 1 to 9, wherein said plurality of nucleic acid binding moieties comprises psoralen.

11. The composition of any one of claims 1 to 10, wherein said dendrimer further comprises an affinity tag.

12. The composition of claim 11, wherein said affinity tag comprises biotin.

13. The composition of any one of claims 1 to 12, wherein said plurality of nucleic acid fragments further comprises an adaptor.

14. The composition of any one of claims 1 to 13, wherein said plurality of nucleic acid fragments are fragments of chromosomal deoxyribonucleic acid (DNA).

15. The composition of any one of claims 1 to 14, wherein said plurality of nucleic acid fragments further comprise barcodes.

16. The composition of any one of claims 1 to 15, wherein said barcodes comprise dibenzocyclooctyl (DBCO) modified nucleotides.

17. The composition of claim 16, wherein said DBCO modified nucleotides are linked to a biotin azide.

18. The composition of claim 17, wherein said DBCO modified nucleotides are linked to a biotin azide via a photocleavable linkage.

19. The composition of claim 17 or claim 18, further comprising a streptavidin bead.

20. The composition of any one of claims 1 to 14, further comprising an endonuclease.

21. The composition of any one of claims 1 to 14, further comprising a ligase.

22. A method of nucleic acid processing, comprising:
(a) obtaining a stabilized sample comprising a nucleic acid molecule complexed to at least one nucleic acid binding protein;
(b) contacting said stabilized sample to a dendrimer comprising a plurality of nucleic acid binding moieties such that said nucleic acid molecule forms a complex with said plurality of nucleic acid binding moieties;
(c) contacting said complex to an endonuclease to cleave said nucleic acid molecule between contact points of said nucleic acid molecule and said plurality of nucleic acid binding moieties creating a plurality of fragments of said nucleic acid molecule each complexed with a nucleic acid binding moiety of said plurality of said nucleic acid binding moieties;
(d) isolating said product of (c) using an agent that binds to said dendrimer;
(e) joining said plurality of fragments to each other to create a concatemer comprising each of said plurality of fragments of said nucleic acid molecule; and
(f) isolating said concatemer from said dendrimer.

23. The method of claim 22, wherein said stabilized sample comprises cross-linked chromatin.

24. The method of claim 22 or claim 23, wherein said stabilized sample comprises cross-linked nuclei.

25. The method of any one of claims 22 to 24, wherein said plurality of nucleic acid binding proteins comprises a histone, a transcription factor, or combination thereof.

26. The method of any one of claims 22 to 25, wherein said plurality of nucleic acid binding moieties comprises a nucleic acid intercalator.

27. The method of any one of claims 22 to 26, wherein said plurality of nucleic acid binding moieties comprises psoralen, chlormethine, cyclophosphamide, chlorambucil, uramustine, melphalan, bendamustine, bis(2-chloroethyl)ethylamine, bis(2-chloroethyl)methylamine, tris(2-chloroethyl)amine, isofamide, carmustine, lomustine, streptozocin, busulfan, cisplatin, carboplatin, cicycloplatin, eptaplatin, lobaplatin, miriplatin, nedaplatin, oxaliplatin, picoplatin, satraplatin, triplatin tetranitrate, procarbazine, altretamine, dacarbazine, mitozolomide, temozolomide, mitomycin C, nitrous acid, formaldehyde, acetylaldehyde, doxorubicin, daunorubicin, epirubicin, or idarubicin.

28. The method of any one of claims 22 to 27, wherein said dendrimer comprises poly(amidoamine) (PAMAM).

29. The method of any one of claims 22 to 28, wherein said dendrimer comprises about 16 to about 512 nucleic acid binding moieties.

30. The method of any one of claims 22 to 29, wherein said endonuclease comprises DNase I, DNase II, micrococcal nuclease, a restriction endonuclease, or a combination thereof.

31. The method of any one of claims 22 to 30, wherein said nucleic acid molecule is a chromosome.

32. The method of any one of claims 22 to 31, wherein said dendrimer further comprises an affinity tag.

33. The method of claim 32, wherein said affinity tag comprises biotin.

34. The method of claim 32 or claim 33, wherein said agent binds to said affinity tag.

35. The method of any one of claims 22 to 34, further comprising subsequent to step (d) and prior to step (e), contacting said product of (c) to a plurality of oligonucleotides and joining each fragment of said plurality of fragments to one of said plurality of oligonucleotides.

36. The method of claim 35, wherein said plurality of oligonucleotides comprise barcodes.

37. The method of claim 35 or claim 36, wherein said plurality of oligonucleotides comprise dibenzocyclooctyl (DBCO) modified nucleotides.

38. The method of claim 37, further comprising forming a linkage between said DBCO to a biotin azide.

39. The method of claim 38, wherein said linkage is photocleavable.

40. The method of claim 38 or claim 39, wherein a streptavidin bead is used to isolate said concatemer from said dendrimer.

41. The method of any one of claims 22 to 40, wherein said stabilized sample comprises a single cell.

42. The method of any one of claims 22 to 40, wherein said stabilized sample comprises a formalin-fixed paraffin embedded (FFPE) sample.

43. The method of any one of claims 22 to 40, wherein said stabilized sample comprises cell-free nucleic acids.

44. The method of any one of claims 22 to 40, wherein said stabilized sample comprises a microbiome.

45. The method of any one of claims 22 to 44, further comprising obtaining a sequence of said concatemer.

46. A method of determining long range phase information, the method comprising:
(a) obtaining a stabilized sample comprising a nucleic acid molecule complexed to at least one nucleic acid binding protein;
(b) contacting said stabilized sample to a dendrimer comprising a plurality of nucleic acid binding moieties such that said nucleic acid molecule forms a complex with said plurality of nucleic acid binding moieties;
(c) contacting said complex to an endonuclease to cleave said nucleic acid molecule between contact points of said nucleic acid molecule and said plurality of nucleic acid binding moieties creating a plurality of fragments of said nucleic acid molecule each complexed with a nucleic acid binding moiety of said plurality of said nucleic acid binding moieties;
(d) isolating said product of (c) using an agent that binds to said dendrimer;
(e) joining said plurality of fragments to each other to create a concatemer comprising each of said plurality of fragments of said nucleic acid molecule;
(f) isolating said concatemer from said dendrimer; and
(g) obtaining a sequence of said concatemer, wherein said sequence comprises said long range phase information.

47. The method of claim 46, wherein said stabilized sample comprises cross-linked chromatin.

48. The method of claim 46 or claim 47, wherein said stabilized sample comprises cross-linked nuclei.

49. The method of any one of claims 46 to 48, wherein said plurality of nucleic acid binding proteins comprises a histone, a transcription factor, or a combination thereof.

50. The method of any one of claims 46 to 49, wherein said plurality of nucleic acid binding moieties comprises a nucleic acid intercalator.

51. The method of any one of claims 46 to 50, wherein said plurality of nucleic acid binding moieties comprises psoralen, chlormethine, cyclophosphamide, chlorambucil, uramustine, melphalan, bendamustine, bis(2-chloroethyl)ethylamine, bis(2-chloroethyl)methylamine, tris(2-chloroethyl)amine, isofamide, carmustine, lomustine, streptozocin, busulfan, cisplatin, carboplatin, cicycloplatin, eptaplatin, lobaplatin, miriplatin, nedaplatin, oxaliplatin, picoplatin, satraplatin, triplatin tetranitrate, procarbazine, altretamine, dacarbazine, mitozolomide, temozolomide, mitomycin C, nitrous acid, formaldehyde, acetylaldehyde, doxorubicin, daunorubicin, epirubicin, or idarubicin.

52. The method of any one of claims 46 to 51, wherein said dendrimer comprises poly(amidoamine) (PAMAM).

53. The method of any one of claims 46 to 52, wherein said dendrimer comprises about 16 to about 512 nucleic acid binding moieties.

54. The method of any one of claims 46 to 53, wherein said endonuclease comprises DNase I, DNase II, micrococcal nuclease, a restriction endonuclease, or a combination thereof.

55. The method of any one of claims 46 to 54, wherein said nucleic acid molecule is a chromosome.

56. The method of any one of claims 46 to 55, wherein said dendrimer further comprises an affinity tag.

57. The method of claim 56, wherein said affinity tag comprises biotin.

58. The method of claim 56 or claim 57, wherein said agent binds to said affinity tag.

59. The method of any one of claims 46 to 58, further comprising subsequent to step (d) and prior to step (e), contacting said product of (c) to a plurality of oligonucleotides and joining each fragment of said plurality of fragments to one of said plurality of oligonucleotides.

60. The method of claim 59, wherein said plurality of oligonucleotides comprise barcodes.

61. The method of claim 59 or claim 60, wherein said plurality of oligonucleotides comprise dibenzocyclooctyl (DBCO) modified nucleotides.

62. The method of claim 61, further comprising forming a linkage between said DBCO to a biotin azide.

63. The method of claim 62, wherein said linkage is photocleavable.

64. The method of claim 62 or claim 63, wherein a streptavidin bead is used to isolate said concatemer from said dendrimer.

65. The method of any one of claims 46 to 64, wherein said stabilized sample comprises a single cell.

66. The method of any one of claims 46 to 64, wherein said stabilized sample comprises a formalin-fixed paraffin embedded (FFPE) sample.

67. The method of any one of claims 46 to 64, wherein said stabilized sample comprises a microbiome.

68. A method of nucleic acid processing, comprising:
(a) obtaining a stabilized sample comprising a nucleic acid molecule complexed to at least one nucleic acid binding protein;
(b) contacting said stabilized sample to an endonuclease to create fragments of said nucleic acid molecule;
(c) contacting said fragments to a plurality of dendrimers comprising a plurality of oligonucleotides, wherein each dendrimer of said plurality of dendrimers comprises a unique barcode sequence and a constant sequence;
(d) joining said each of said fragments to at least one of said plurality of oligonucleotides of said plurality of dendrimers to form a complex;
(e) isolating said complex of (d) using an agent that binds to said plurality of dendrimers; and
(f) isolating said fragments joined to said oligonucleotides from said dendrimer.

69. The method of claim 68, wherein said stabilized sample comprises cross-linked chromatin.

70. The method of claim 68 or claim 69, wherein said stabilized sample comprises cross-linked nuclei.

71. The method of any one of claims 68 to 70, wherein said nucleic acid binding protein comprises a histone, a transcription factor, or a combination thereof.

72. The method of any one of claims 68 to 71, wherein said plurality of dendrimers are coupled to a solid surface.

73. The method of any one of claim 72, wherein said solid surface is a bead.

74. The method of any one of claims 68 to 73, wherein said plurality of dendrimers comprises poly(amidoamine) (PAMAM).

75. The method of any one of claims 68 to 74, wherein each of said plurality of dendrimers comprises about 16 to about 512 nucleic acid binding moieties.

76. The method of any one of claims 68 to 75, wherein said endonuclease DNase I, DNase II, micrococcal nuclease, a restriction endonuclease, or a combination thereof.

77. The method of any one of claims 68 to 76, wherein said nucleic acid molecule is a chromosome.

78. The method of any one of claims 68 to 77, wherein said plurality of dendrimers further comprises an affinity tag.

79. The method of claim 78, wherein said affinity tag comprises biotin.

80. The method of claim 78 or claim 79, wherein said agent binds to said affinity tag.

81. The method of any one of claims 68 to 80, wherein said stabilized sample comprises a single cell.

82. The method of any one of claims 68 to 80, wherein said stabilized sample comprises a formalin-fixed paraffin embedded (FFPE) sample.

83. The method of any one of claims 68 to 80, wherein said stabilized sample comprises cell-free nucleic acids.

84. The method of any one of claims 68 to 80, wherein said stabilized sample comprises a microbiome.

85. The method of any one of claims 68 to 84, further comprising obtaining plurality of sequence reads of said plurality of fragments joined to said oligonucleotides.

86. A method of single cell genomic analysis, the method comprising:
(a) contacting a stabilized nucleus comprising a plurality of nucleic acid molecules bound to at least one nucleic acid binding protein of said single cell an endonuclease to create a plurality of nucleic acid fragments;
(b) contacting said plurality of nucleic acid fragments to a dendrimer comprising a plurality of oligonucleotides to link said plurality of nucleic acid fragments to said plurality of oligonucleotides, wherein said plurality of oligonucleotides each comprise a barcode sequence and a constant sequence;
(c) isolating said dendrimer linked to said plurality of oligonucleotides linked to said plurality of nucleic acid fragments from said nucleus using an agent that binds to said dendrimer; and
(e) sequencing said plurality of oligonucleotides linked to said plurality of nucleic acid fragments.

87. The method of claim 86, wherein said nucleic acid binding protein comprises a histone, a transcription factor, or a combination thereof.

88. The method of claim 86 or claim 87, wherein dendrimer is coupled to a solid surface.

89. The method of claim 88, wherein said solid surface is a bead.

90. The method of any one of claims 86 to 89, wherein said dendrimer comprises poly(amidoamine) (PAMAM).

91. The method of any one of claims 86 to 90, wherein said dendrimer comprises about 16 to about 512 nucleic acid binding moieties.

92. The method of any one of claims 86 to 91, wherein said endonuclease comprises DNase I, DNase II, micrococcal nuclease, a restriction endonuclease, or a combination thereof.

93. The method of any one of claims 86 to 92, wherein said plurality of nucleic acid molecules are chromosomes.

94. The method of any one of claims 86 to 93, wherein said dendrimer further comprises an affinity tag.

95. The method of claim 94, wherein said affinity tag comprises biotin.

96. The method of claim 94 or claim 95, wherein said agent binds to said affinity tag.

97. A method of processing a sample comprising cell-free nucleic acids, the method comprising:
(a) contacting a sample comprising a plurality of cell-free nucleic acids to a dendrimer comprising a plurality of nucleic acid binding moieties such that said plurality of cell-free nucleic acids forms a complex with said plurality of nucleic acid binding moieties;
(b) isolating said complex of (a) using an agent that binds to said dendrimer;
(c) joining said plurality of fragments to each other to create a concatemer comprising each of said plurality of cell-free nucleic acids; and
(d) isolating said concatemer from said dendrimer.

98. The method of claim 97, wherein said plurality of nucleic acid binding moieties comprises a nucleic acid intercalator.

99. The method of claim 97 or claim 98, wherein said plurality of nucleic acid binding moieties comprises psoralen, chlormethine, cyclophosphamide, chlorambucil, uramustine, melphalan, bendamustine, bis(2-chloroethyl)ethylamine, bis(2-chloroethyl)methylamine, tris(2-chloroethyl)amine, isofamide, carmustine, lomustine, streptozocin, busulfan, cisplatin, carboplatin, cicycloplatin, eptaplatin, lobaplatin, miriplatin, nedaplatin, oxaliplatin, picoplatin, satraplatin, triplatin tetranitrate, procarbazine, altretamine, dacarbazine, mitozolomide, temozolomide, mitomycin C, nitrous acid, formaldehyde, acetylaldehyde, doxorubicin, daunorubicin, epirubicin, or idarubicin.

100. The method of any one of claims 97 to 99, wherein said dendrimer comprises poly(amidoamine) (PAMAM).

101. The method of any one of claims 97 to 100, wherein said dendrimer comprises about 16 to about 512 nucleic acid binding moieties.

102. The method of any one of claims 97 to 101, wherein said dendrimer further comprises an affinity tag.

103. The method of claim 102, wherein said affinity tag comprises biotin.

104. The method of claim 102 or claim 103, wherein said agent binds to said affinity tag.

105. The method of any one of claims 97 to 104, further comprising subsequent to step (c) and prior to step (d), contacting said product of (a) to a plurality of oligonucleotides and joining each fragment of said plurality of fragments to one of said plurality of oligonucleotides.

106. The method of claim 105, wherein said plurality of oligonucleotides comprise barcodes.

107. The method of claim 105 or claim 106, wherein said plurality of oligonucleotides comprise dibenzocyclooctyl (DBCO) modified nucleotides.

108. The method of claim 107, further comprising forming a linkage between said DBCO to a biotin azide.

109. The method of claim 108, wherein said linkage is photocleavable.

110. The method of claim 108 or claim 109, wherein a streptavidin bead is used to isolate said concatemer from said dendrimer.

111. The method of any one of claims 97 to 110, further comprising obtaining a sequence of said concatemer.

112. A method of processing a metagenomic sample, the method comprising:
(a) obtaining a stabilized sample comprising a plurality of nucleic acid molecules from a plurality of organisms, wherein each nucleic acid molecule of said plurality is complexed to at least one nucleic acid binding protein;
(b) contacting said stabilized sample to a plurality of dendrimers each dendrimer comprising a plurality of nucleic acid binding moieties such that each of said plurality of nucleic acid molecules forms a complex with said plurality of nucleic acid binding moieties of at least one of said plurality of dendrimers resulting in a plurality of complexes;
(c) contacting said plurality of complexes to an endonuclease to cleave said plurality of nucleic acid molecules between contact points of said nucleic acid molecule and said plurality of nucleic acid binding moieties creating a plurality of fragments of each of said plurality of nucleic acid molecules each fragment of said plurality of fragments complexed with a nucleic acid binding moiety of said plurality of said nucleic acid binding moieties;
(d) isolating said product of (c) using an agent that binds to said dendrimer;
(e) joining said plurality of fragments of each complex to each other to create a concatemer comprising each of said plurality of fragments bound to said dendrimer;
(f) isolating said plurality of concatemers from each of said plurality of dendrimers; and
(g) obtaining a plurality of sequences of each of said plurality of concatemers;
wherein each sequence of said plurality of sequences comprises sequence information from an organism of said plurality of organisms.

113. The method of claim 112, wherein said plurality of nucleic acid binding proteins comprises a histone, a transcription factor, or a combination thereof.

114. The method of claim 112 or claim 113, wherein said plurality of nucleic acid binding moieties comprises a nucleic acid intercalator.

115. The method of any one of claims 112 to 114, wherein said plurality of nucleic acid binding moieties comprises psoralen, chlormethine, cyclophosphamide, chlorambucil, uramustine, melphalan, bendamustine, bis(2-chloroethyl)ethylamine, bis(2-chloroethyl)methylamine, tris(2-chloroethyl)amine, isofamide, carmustine, lomustine, streptozocin, busulfan, cisplatin, carboplatin, cicycloplatin, eptaplatin, lobaplatin, miriplatin, nedaplatin, oxaliplatin, picoplatin, satraplatin, triplatin tetranitrate, procarbazine, altretamine, dacarbazine, mitozolomide, temozolomide, mitomycin C, nitrous acid, formaldehyde, acetylaldehyde, doxorubicin, daunorubicin, epirubicin, or idarubicin.

116. The method of any one of claims 112 to 115, wherein said dendrimer comprises poly(amidoamine) (PAMAM).

117. The method of any one of claims 112 to 116, wherein said dendrimer comprises about 16 to about 512 nucleic acid binding moieties.

118. The method of any one of claims 112 to 117, wherein said endonuclease comprises DNase I, DNase II, micrococcal nuclease, a restriction endonuclease, or a combination thereof.

119. The method of any one of claims 112 to 118, wherein said plurality of nucleic acid molecules are chromosomes.

120. The method of any one of claims 112 to 119, wherein said dendrimer further comprises an affinity tag.

121. The method of claim 120, wherein said affinity tag comprises biotin.

122. The method of claim 120 or claim 121, wherein said agent binds to said affinity tag.

123. The method of any one of claims 112 to 122, further comprising subsequent to step (d) and prior to step (b), contacting said product of (e) to a plurality of oligonucleotides and joining each fragment of said plurality of fragments to one of said plurality of oligonucleotides.

124. The method of claim 123, wherein said plurality of oligonucleotides comprise barcodes.

125. The method of claim 123 or claim 124, wherein said plurality of oligonucleotides comprise dibenzocyclooctyl (DBCO) modified nucleotides.

126. The method of claim 125, further comprising forming a linkage between said DBCO to a biotin azide.

127. The method of claim 126, wherein said linkage is photocleavable.

128. The method of claim 126 or claim 127, wherein a streptavidin bead is used to isolate said concatemer from said dendrimer.

129. The method of any one of claims 112 to 128, wherein said stabilized sample comprises a microbiome.

130. The method of any one of claims 112 to 129, further comprising obtaining a sequence of said concatemer.

131. A method of spatial genomic analysis, the method comprising:
(a) contacting a stabilized biological sample comprising a plurality of nucleic acid molecules bound to a nucleic acid binding protein to an endonuclease to create a plurality of nucleic acid fragments in the stabilized biological sample, wherein said stabilized biological sample is attached to a surface;
(b) contacting a plurality of dendrimers, each of said plurality of dendrimers comprising a unique barcode, to said biological sample, wherein each of said plurality of dendrimers binds to a unique position on said surface;
(b) sequencing each unique barcode of said plurality of dendrimers bound to said unique position on said surface;
(c) obtaining location information for each of said plurality of dendrimers bound to said unique position on said surface;
(d) creating a plurality of complexes each comprising a linkage between said plurality of nucleic acid fragments to said plurality of dendrimers bound to said unique position on said surface;
(e) isolating said plurality of complexes of (d); and
(f) obtaining sequence information of said plurality of nucleic acid fragments and said barcodes of said plurality of complexes.

132. The method of claim 131, wherein said plurality of dendrimers are coupled to a bead.

133. The method of claim 131 or claim 132, wherein said dendrimer comprises poly(amidoamine) (PAMAM).

134. The method of any one of claims 131 to 133, wherein said dendrimer comprises about 16 to about 512 nucleic acid binding moieties.

135. The method of any one of claims 131 to 134, wherein said endonuclease comprises DNase I, DNase II, micrococcal nuclease, a restriction endonuclease, or a combination thereof.

136. The method of any one of claims 131 to 135, wherein said dendrimer further comprises an affinity tag.

137. The method of claim 136, wherein said affinity tag comprises biotin.

138. The method of claim 136 or claim 137, wherein said agent binds to said affinity tag.

139. The method of any one of claims 131 to 138, wherein said stabilized biological sample comprises a formalin-fixed paraffin embedded (FFPE) sample.

140. The method of any one of claims 131 to 138, wherein said stabilized biological sample comprises a section of a tissue sample.

141. The method of any one of claims 131 to 138, wherein said stabilized biological sample comprises cultured cells.

142. The method of any one of claims 131 to 141, further comprising obtaining plurality of sequence reads of said plurality of nucleic acid fragments.

143. The method of any one of claims 131 to 141, wherein each of said plurality of fragments are linked to said barcode of said complex.

144. The method of claim 143, further comprising obtaining a sequence of each of said plurality of fragments linked to said barcode.

145. A method comprising:
(a) obtaining a stabilized biological sample comprising a nucleic acid molecule complexed to at least one nucleic acid binding protein;
(b) contacting the nucleic acid molecule with a dendrimer to form a complex, wherein one or more polymers of the dendrimer comprise a terminal primary amine;
(c) cleaving the nucleic acid molecule into a plurality of segments comprising at least a first segment and a second segment; and
(d) attaching the first segment and the second segment of the plurality of segments at a junction.

146. The method of claim 145, wherein the dendrimer is modified with a crosslinker.

147. The method of claim 145, further comprising, prior to (b) contacting the dendrimer with a crosslinker.

148. The method of claim 146 or claim 147, wherein the crosslinker comprises psoralen, chlormethine, cyclophosphamide, chlorambucil, uramustine, melphalan, bendamustine, bis(2-chloroethyl)ethylamine, bis(2-chloroethyl)methylamine, tris(2-chloroethyl)amine, isofamide, carmustine, lomustine, streptozocin, busulfan, cisplatin, carboplatin, cicycloplatin, eptaplatin, lobaplatin, miriplatin, nedaplatin, oxaliplatin, picoplatin, satraplatin, triplatin tetranitrate, procarbazine, altretamine, dacarbazine, mitozolomide, temozolomide, mitomycin C, nitrous acid, formaldehyde, acetylaldehyde, doxorubicin, daunorubicin, epirubicin, or idarubicin.

149. The method of claim 148, wherein the psoralen comprises an N-hydroxysuccinimide (NHS) ester-conjugated psoralen.

150. The method of any one of claims 145 to 149, wherein the dendrimer comprises a polyamidoamine (PAMAM) dendrimer

151. The method of any one of claims 145 to 150, further comprising: (e) uncoupling the crosslinker from the dendrimer.

152. The method of claim 151, wherein the uncoupling comprises a hot alkali treatment.

153. The method of claim 151, wherein the uncoupling comprises exposure to UV radiation.

154. The method of any one of claims 145 to 153, wherein a portion of the plurality of segments are joined to form concatemers.

155. The method of claim 154, wherein the concatemers comprise at least three segments.

156. The method of claim 154, wherein the concatemers comprise at least four segments.

157. The method of claim 154, wherein the concatemers comprise at least five segments.

158. The method of claim 154, wherein the concatemers comprise at least six segments.

159. The method of claim 154, wherein the concatemers comprise at least eight segments.

160. The method of claim 154, wherein the concatemers comprise at least ten segments.

161. The method of any one of claims 145 to 160, wherein the dendrimer has a molecular weight of from 5 kilodaltons (kDa) to 125 kDa.

162. The method of any one of claims 145 to 161, wherein the dendrimer has a molecular weight of from 6 kDa to 8 kDa.

163. The method of any one of claims 145 to 161, wherein the dendrimer has a molecular weight of from 25 kDa to 35 kDa.

164. The method of any one of claims 145 to 161, wherein the dendrimer has a molecular weight of from 110 kDa to 125 kDa.

165. The method of any one of claims 145 to 164, wherein the dendrimer comprises from 32 to 512 reactive groups.

166. The method of any one of claims 145 to 165, wherein the dendrimer comprises about 32 reactive groups.

167. The method of any one of claims 145 to 165, wherein the dendrimer comprises about 128 reactive groups.

168. The method of any one of claims 145 to 165, wherein the dendrimer comprises about 512 reactive groups

169. The method of any one of claims 145 to 168, further comprising, subsequent to (b), photoactivating the dendrimer complex.

170. The method of any one of claims 145 to 169, further comprising (f) subjecting the plurality of segments to size selection to obtain a plurality of selected segments.

171. The method of any one of claims 145 to 170, wherein the cleaving comprises contacting the nucleic acid molecule with a deoxyribonuclease (DNase).

172. The method of claim 171, wherein the DNase comprises DNase I, DNase II, micrococcal nuclease, a restriction endonuclease, or a combination thereof.

173. The method of any one of claim 145 to 172, wherein the stabilized biological sample has been treated with a crosslinking agent.

174. The method of claim 173, wherein the crosslinking agent is a chemical fixative.

175. The method of claim 174, wherein the chemical fixative comprises formaldehyde, psoralen, disuccinimidyl glutarate (DSG), ethylene glycol bis(succinimidyl succinate) (EGS), ultraviolet light, or a combination thereof.

176. The method of claim 173, wherein the crosslinking agent comprises chlormethine, cyclophosphamide, chlorambucil, uramustine, melphalan, bendamustine, bis(2-chloroethyl)ethylamine, bis(2-chloroethyl)methylamine, tris(2-chloroethyl)amine, isofamide, carmustine, lomustine, streptozocin, busulfan, cisplatin, carboplatin, cicycloplatin, eptaplatin, lobaplatin, miriplatin, nedaplatin, oxaliplatin, picoplatin, satraplatin, triplatin tetranitrate, procarbazine, altretamine, dacarbazine, mitozolomide, temozolomide, mitomycin C, nitrous acid, formaldehyde, acetylaldehyde, doxorubicin, daunorubicin, epirubicin, or idarubicin.

177. The method of any one of claims 145 to 176, wherein the stabilized biological sample is a crosslinked paraffin-embedded tissue sample.

178. The method of any one of claims 145 to 176, wherein the stabilized biological sample comprises a stabilized cell lysate.

179. The method of any one of claims 145 to 176, wherein the stabilized biological sample comprises a stabilized intact cell.

180. The method of any one of claims 145 to 176, wherein the stabilized biological sample comprises a stabilized intact nucleus.

181. The method of claim 179 or claim 180, wherein step (c) is conducted prior to lysis of the intact cell or the intact nucleus.

182. The method of any one of claims 145 to 178, further comprising, prior to step (d), lysing cells and/or nuclei in the stabilized biological sample.

183. The method of any one of claims 145 to 182, wherein the stabilized biological sample comprises fewer than 3,000,000 cells.

184. The method of any one of claims 145 to 182, wherein the stabilized biological sample comprises fewer than 1,000,000 cells.

185. The method of any one of claims 145 to 182, wherein the stabilized biological sample comprises fewer than 100,000 cells.

186. The method of any one of claims 145 to 185, wherein the attaching comprises filling in sticky ends using biotin tagged nucleotides and ligating blunt ends.

187. The method of any one of claims 145 to 185, wherein the attaching comprises contacting at least the first segment and the second segment to at least one bridge oligonucleotide.

188. The method of claim 187, wherein the bridge oligonucleotide comprises a barcode sequence.

189. The method of claim 188, wherein the attaching comprises contacting at least the first segment and the second segment to multiple bridge oligonucleotides in series.

190. The method of claim 187, wherein the attaching results in samples, cells, nuclei, chromosomes, or nucleic acid molecules of the stabilized biological sample receiving a unique sequence of bridge oligonucleotides.

191. The method of any one of claims 145 to 185, wherein attaching comprises contacting at least the first segment and the second segment to a barcode.

192. The method of any one of claims 145 to 191, further comprising:
(g) obtaining at least some sequence on each side of the junction to generate a first read pair.

193. The method of claim 192, further comprising:
(h) mapping the first read pair to a set of contigs; and
(i) determining a path through the set of contigs that represents an order and/or orientation to a genome.

194. The method of claim 192, further comprising:
(h) mapping the first read pair to a set of contigs; and
(i) determining, from the set of contigs, a presence of a structural variant or loss of heterozygosity in the stabilized biological sample.

195. The method of claim 192, further comprising:
(h) mapping the first read pair to a set of contigs; and
(i) assigning a variant in the set of contigs to a phase.

196. The method of claim 192, further comprising:
(h) mapping the first read pair to a set of contigs;
(i) determining, from the set of contigs, a presence of a variant in the set of contigs; and
(j) conducting a step selected from one or more of: (1) identifying a disease stage, a prognosis, or a course of treatment for the stabilized biological sample; (2) selecting a drug based on the presence of the variant; or (3) identifying a drug efficacy for the stabilized biological sample.
